(19) United States
(12) Patent Application Publication
(10) Pub. No.: US 2023/0279001 A1
(43) Pub. Date: Sep. 7, 2023

(54) IMIDAZO[1,2-A]PYRIDINE COMPOUNDS FOR THE TREATMENT OF AUTOIMMUNE DISEASE

(71) Applicant: Hoffmann-La Roche Inc., Little Falls, NJ (US)

(72) Inventors: Dongdong CHEN, Shanghai (CN); Jianguo CHEN, Shanghai (CN); Fabian DEY, Zürich (CH); Xin HONG, Shanghai (CN); Xuefei TAN, Shanghai (CN); Jiasu XU, Shanghai (CN); Weixing ZHANG, Shanghai (CN); Wei ZHU, Shanghai (CN); Ge ZOU, Shanghai (CN)

(73) Assignee: Hoffmann-La Roche Inc., Little Falls, NJ (US)

(21) Appl. No.: 18/040,200

(22) PCT Filed: Aug. 2, 2021
(86) PCT No.: PCT/EP2021/071552

§ 371 Date: Feb. 1, 2023

(30) Foreign Application Priority Data
Aug. 4, 2020 (WO) PCT/CN2020/106796

(51) Int. Cl. C07D 471/04; C07D 519/00

(52) U.S. Cl. CPC C07D 471/04; C07D 519/00

(57) ABSTRACT

The present invention relates to compounds of formula (I), wherein R¹, R², R³, R⁴, R⁵ and A are as described herein, and their pharmaceutically acceptable salt thereof, and compositions including the compounds and methods of using the compounds.

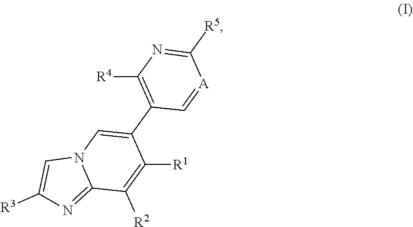

The present invention relates to organic compounds useful for therapy and/or prophylaxis in a mammal, and in particular to antagonist of TLR7 and/or TLR8 and/or TLR9 useful for treating systemic lupus erythematosus or lupus nephritis.

### FIELD OF THE INVENTION

Autoimmune connective tissue disease (CTD) include prototypical autoimmune syndromes such as Systemic Lupus Erythematosus (SLE), primary Sjögren's syndrome (pSjS), mixed connective tissue disease (MCTD), Dermatomyositis/Polymyositis (DM/PM), Rheumatoid Arthritis (RA), and systemic sclerosis (SSc). With the exception of RA, no really effective and safe therapies are available to patients. SLE represents the prototypical CTD with a prevalence of 20-150 per 100,000 and causes broad inflammation and tissue damage in distinct organs, from commonly observed symptoms in the skin and joints to renal, lung, or heart failure. Traditionally, SLE has been treated with nonspecific anti-inflammatory or immunosuppressive drugs. However, long-term usage of immunosuppressive drug, e.g. corticosteroids is only partially effective, and is associated with undesirable toxicity and side effects. Belimumab is the only FDA-approved drug for lupus in the last 50 years, despite its modest and delayed efficacy in only a fraction of SLE patients (Navarra, S. V. et al Lancet 2011, 377, 721). Other biologics, such as anti-CD20 mAbs, mAbs against or soluble receptors of specific cytokines, have failed in most clinical studies. Thus, novel therapies are required that provide sustained improvement in a greater proportion of patient groups and are safer for chronic use in many autoimmune as well as autoinflammation diseases.

Toll like Receptors (TLR) are an important family of pattern recognition receptors (PRR) which can initiate broad immune responses in a wide variety of immune cells. As natural host defense sensors, endosomal TLRs 7, 8 and 9 recognize nucleic acids derived from viruses, bacteria; specifically, TLR7/8 and TLR9 recognize single-stranded RNA (ssRNA) and single-stranded CpG-DNA, respectively. However, aberrant nucleic acid sensing of TRL7, 8, 9 is considered as a key node in a broad of autoimmune and auto-inflammatory diseases (Krieg, A. M. et al. Immunol. Rev. 2007, 220, 251. Jimenez-Dalmaroni, M. J. et al Autoimmun Rev. 2016, 15, 1. Chen, J. Q., et al. Clinical Reviews in Allergy & Immunology 2016, 50, 1). Anti-RNA and anti-DNA antibodies are well-established diagnostic markers of SLE, and these antibodies can deliver both self-RNA and self-DNA to endosomes. While self-RNA complexes can be recognized by TLR7 and TLR8, self-DNA complexes can trigger TLR9 activation. Indeed, defective clearance of self-RNA and self-DNA from blood and/or tissues is evident in SLE (Systemic Lupus Erythematosus) patients. TLR7 and TLR9 have been reported to be upregulated in SLE tissues, and correlate with chronicity and activity of lupus nephritis, respectively. In B cells of SLE patients, TLR7 expression correlates with anti-RNP antibody production, while TLR9 expression with IL-6 and anti-dsDNA antibody levels. Consistently, in lupus mouse models, TLR7 is required for anti-RNA antibodies, and TLR9 is required for anti-nucleosome antibody. On the other hand, overexpression of TLR7 or human TLR8 in mice promotes autoimmunity and autoinflammation. Moreover, activation of TLR8 specifically contributes to inflammatory cytokine secretion of mDC/macrophages, neutrophil NETosis, induction of Th17 cells, and suppression of Treg cells. In addition to the described role of TLR9 in promoting autoantibody production of B cells, activation of TLR9 by self-DNA in pDC also leads to induction of type I IFNs and other inflammatory cytokines. Given these roles of TLR9 in both pDC and B cells, both as key contributors to the pathogenesis of autoimmune diseases, and the extensive presence of self-DNA complexes that could readily activate TLR9 in many patients with autoimmune diseases, it may have extra benefit to further block self-DNA mediated TLR9 pathways on top of inhibition of TLR7 and TLR8 pathways. Taken together, TLR7, 8 and 9 pathways represent new therapeutic targets for the treatment of autoimmune and auto-inflammatory diseases, for which no effective steroid-free and non-cytotoxic oral drugs exist, and inhibition of all these pathways from the very upstream may deliver satisfying therapeutic effects. As such, we invented oral compounds that target and suppress TLR7, TLR8 and TLR9 for the treatment of autoimmune and auto-inflammatory diseases.

### SUMMARY OF THE INVENTION

The present invention relates to novel compounds of formula (I),

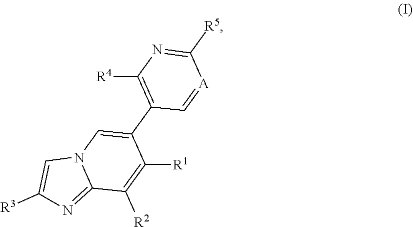

wherein
R¹ is H or C₁₋₆ alkyl;
R² is C₁₋₆ alkyl or C₁₋₆alkoxy;
R³ is H or C₁₋₆ alkyl;
R⁴ is H, C₁₋₆ alkyl, haloC₁₋₆alkyl, C₁₋₆alkoxy or (C₁₋₆ alkyl)₂amino;
R⁵ is (5,6,7,8-tetrahydropyrido[3,4-d]pyrimidinyl)piperazinyl; (amino(C₁₋₆alkoxy)pyrrolidinyl)piperidinyl; (C₁₋₆alkylpiperazinyl)piperidinyl; (hydroxyC₁₋₆alkyl)piperazinyl; (morpholinylcarbonyl)piperazinyl; 1,3,4,6,7,8,9,9a-octahydropyrazino[1,2-a]pyrazinyl; 3,9-diazaspiro[5.5]undecanyl; 4-oxa-1,9-diazaspiro[5.5]undecanyl; 5-oxa-2,8-diazaspiro[3.5]nonanyl; amino(C₁₋₆alkoxy)pyrrolidinyl; amino(C₁₋₆alkyl)azetidinyl; amino(C₁₋₆ alkyl)piperidinyl; aminopiperidinyl or piperazinylpiperidinyl;
A is CH, N or CR⁶, wherein R⁶ is C₁₋₆ alkyl;
   or a pharmaceutically acceptable salt thereof.

Another object of the present invention is related to novel compounds of formula (I). Their manufacture, medicaments based on a compound in accordance with the invention and their production as well as the use of compounds of formula (I) as TLR7 and TLR8 and TLR9 antagonist, and for the treatment or prophylaxis of systemic lupus erythematosus or lupus nephritis. The compounds of formula (I) show superior TLR7 and TLR8 and TLR9 antagonism activity. In addition, the compounds of formula (I) also show good cytotoxicity, phototoxicity, solubility, hPBMC, human microsome stability, AO (human cytosolic aldehyde oxidase) and SDPK profiles, as well as low CYP inhibition.

### DETAILED DESCRIPTION OF THE INVENTION

### Definitions

The term “C₁₋₆ alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆ alkyl” groups are methyl, ethyl and n-propyl.

The term “halogen” and “halo” are used interchangeably herein and denote fluoro, chloro, bromo, or iodo.

The term “haloC₁₋₆alkyl” denotes a C₁₋₆ alkyl group wherein at least one of the hydrogen atoms of the C₁₋₆ alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, trifluoroethyl, fluoromethyl, difluoromethyl, difluoroethyl or trifluoromethyl.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Antagonist of TLR7 and TLR8 and TLR9

The present invention relates to (i) a compound of formula (I),

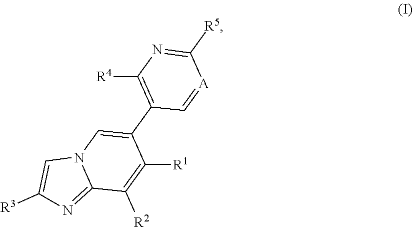

wherein
R¹ is H or C₁₋₆alkyl;
R² is C₁₋₆alkyl or C₁₋₆alkoxy;
R³ is H or C₁₋₆alkyl;
R⁴ is H, C₁₋₆alkyl, haloC₁₋₆alkyl, C₁₋₆alkoxy or (C₁₋₆ alkyl)₂amino;
R⁵ is (5,6,7,8-tetrahydropyrido[3,4-d]pyrimidinyl)piperazinyl; (amino(C₁₋₆alkoxy)pyrrolidinyl)piperidinyl; (C₁₋₆alkylpiperazinyl)piperidinyl; (hydroxyC₁₋₆alkyl)piperazinyl; (morpholinylcarbonyl)piperazinyl; 1,3,4,6,7,8,9,9a-octahydropyrazino[1,2-a]pyrazinyl; 3,9-diazaspiro[5.5]undecanyl; 4-oxa-1,9-diazaspiro[5.5]undecanyl; 5-oxa-2,8-diazaspiro[3.5]nonanyl; amino(C₁₋₆alkoxy)pyrrolidinyl; amino(C₁₋₆alkyl)azetidinyl; amino(C₁₋₆ alkyl)piperidinyl; aminopiperidinyl or piperazinylpiperidinyl;
A is CH, N or CR⁶, wherein R⁶ is C₁₋₆ alkyl;
   or a pharmaceutically acceptable salt thereof.

A further embodiment of present invention is (ii) a compound of formula (I) according to (i), or a pharmaceutically acceptable salt thereof, wherein A is CH or N.

A further embodiment of present invention is (iii) a compound of formula (I) according to (i) or (ii), or a pharmaceutically acceptable salt thereof, wherein R¹ is H.

A further embodiment of present invention is (iv) a compound of formula (I), according to any one of (i) to (iii), or a pharmaceutically acceptable salt thereof, wherein R³ is H.

A further embodiment of present invention is (v) a compound of formula (I) according to any one of (i) to (iv), wherein R⁴ is R⁴ is C₁₋₆ alkyl, haloC₁₋₆alkyl or C₁₋₆alkoxy.

A further embodiment of present invention is (vi) a compound of formula (I), or a pharmaceutically acceptable salt thereof, according to any one of (i) to (v), wherein R⁵ is (amino(C₁₋₆alkoxy)pyrrolidinyl)piperidinyl; 3,9-diazaspiro[5.5]undecanyl; amino(C₁₋₆alkoxy)pyrrolidinyl; piperazinylpiperidinyl or 5-oxa-2,8-diazaspiro[3.5]nonanyl.

A further embodiment of present invention is (vii) a compound of formula (I), or a pharmaceutically acceptable salt thereof, according to any one of (i) to (vi), wherein (3-amino-4-methoxy-pyrrolidin-1-yl)-1-piperidinyl; 3,9-diazaspiro[5.5]undecan-3-yl; 4-methoxy-3-amino-pyrrolidin-1-yl; 4-piperazin-1-yl-1-piperidinyl or 5-oxa-2,8-diazaspiro[3.5]nonan-2-yl.

A further embodiment of present invention is (viii) a compound of formula (I), or a pharmaceutically acceptable salt thereof, according to any one of (i) to (vii), wherein
R¹ is H;
R² is C₁₋₆ alkyl or C₁₋₆alkoxy;
R³ is H;
R⁴ is C₁₋₆ alkyl, haloC₁₋₆alkyl or C₁₋₆alkoxy;
R⁵ is (amino(C₁₋₆alkoxy)pyrrolidinyl)piperidinyl; 3,9-diazaspiro[5.5]undecanyl; amino(C₁₋₆alkoxy)pyrrolidinyl; piperazinylpiperidinyl or 5-oxa-2,8-diazaspiro[3.5]nonanyl;
A is CH or N;
   or a pharmaceutically acceptable salt thereof.

A further embodiment of present invention is (ix) a compound of formula (I), or a pharmaceutically acceptable salt thereof, according to any one of (i) to (viii), wherein
R¹ is H;
R² is methyl or methoxy;
R³ is H;
R⁴ is ethyl, difluoromethyl or methoxy;
R⁵ is (3-amino-4-methoxy-pyrrolidin-1-yl)-1-piperidinyl; 3,9-diazaspiro[5.5]undecan-3-yl; 4-methoxy-3-amino-pyrrolidin-1-yl; 4-piperazin-1-yl-1-piperidinyl or 5-oxa-2,8-diazaspiro[3.5]nonan-2-yl;
A is CH or N;
   or a pharmaceutically acceptable salt thereof.

Another embodiment of present invention is a compound of formula (I) selected from the following:
1-[6-isopropyl-5-(8-methylimidazo[1,2-a]pyridin-6-yl)-2-pyridyl]piperidin-4-amine;
6-[2-isopropyl-6-(4-piperazin-1-yl-1-piperidyl)-3-pyridyl]-8-methyl-imidazo[1,2-a]pyridine;
6-[2-isopropyl-6-(4-piperazin-1-yl-1-piperidyl)-3-pyridyl]-2,8-dimethyl-imidazo[1,2-a]pyridine;
1-[5-(2,8-dimethylimidazo[1,2-a]pyridin-6-yl)-6-isopropyl-2-pyridyl]piperidin-4-amine;
[(2S)-1-[5-(2,8-dimethylimidazo[1,2-a]pyridin-6-yl)-6-isopropyl-2-pyridyl]piperazin-2-yl]methanol;
9-[5-(2,8-dimethylimidazo[1,2-a]pyridin-6-yl)-6-isopropyl-2-pyridyl]-4-oxa-1,9-diazaspiro[5.5]undecane;
6-[2-isopropyl-6-(4-piperazin-1-yl-1-piperidyl)-3-pyridyl]-7,8-dimethyl-imidazo[1,2-a]pyridine;
2-[5-(2,8-dimethylimidazo[1,2-a]pyridin-6-yl)-6-isopropyl-2-pyridyl]-1,3,4,6,7,8,9,9a-octahydropyrazino[1,2-a]pyrazine;
8-methyl-6-[5-methyl-6-[4-(4-methylpiperazin-1-yl)-1-piperidyl]-3-pyridyl]imidazo[1,2-a]pyridine;

6-[2-(difluoromethyl)-6-(4-piperazin-1-yl-1-piperidyl)-3-pyridyl]-8-methyl-imidazo[1,2-a]pyridine;
6-[2-(difluoromethyl)-6-(4-piperazin-1-yl-1-piperidyl)-3-pyridyl]-2,8-dimethyl-imidazo[1,2-a]pyridine;
1-[1-[6-(difluoromethyl)-5-(2,8-dimethylimidazo[1,2-a]pyridin-6-yl)-2-pyridyl]-4-piperidyl]-3-methyl-azetidin-3-amine;
3-[6-(difluoromethyl)-5-(8-methoxyimidazo[1,2-a]pyridin-6-yl)-2-pyridyl]-3,9-diazaspiro[5.5]undecane;
2-[1-[6-ethyl-5-(8-methylimidazo[1,2-a]pyridin-6-yl)-2-pyridyl]-4-piperidyl]-5-oxa-2,8-diazaspiro[3.5]nonane;
(3R,4R)-1-[1-[6-ethyl-5-(8-methylimidazo[1,2-a]pyridin-6-yl)-2-pyridyl]-4-piperidyl]-4-methoxy-pyrrolidin-3-amine;
[4-[5-(2,8-dimethylimidazo[1,2-a]pyridin-6-yl)-6-ethyl-2-pyridyl]-4-piperidyl]-5-oxa-2,8-diazaspiro[3.5]nonane;
(3R,4R)-1-[1-[5-(2,8-dimethylimidazo[1,2-a]pyridin-6-yl)-6-ethyl-2-pyridyl]-4-piperidyl]-4-methoxy-pyrrolidin-3-amine;
[4-[5-(2,8-dimethylimidazo[1,2-a]pyridin-6-yl)-6-ethyl-2-pyridyl]piperazin-1-yl]-morpholin-2-yl-methanone;
2-[4-[5-(2,8-dimethylimidazo[1,2-a]pyridin-6-yl)-6-ethyl-2-pyridyl]piperazin-1-yl]-5,6,7,8-tetrahydropyrido[3,4-d]pyrimidine;
1-[5-(2,8-dimethylimidazo[1,2-a]pyridin-6-yl)-6-ethyl-2-pyridyl]-4-methyl-piperidin-4-amine;
6-[2-ethyl-6-(4-piperazin-1-yl-1-piperidyl)-3-pyridyl]-2,8-dimethyl-imidazo[1,2-a]pyridine;
3-[3-ethyl-5-(8-methylimidazo[1,2-a]pyridin-6-yl)-2-pyridyl]-3,9-diazaspiro[5.5]undecane;
N,N-dimethyl-3-(8-methylimidazo[1,2-a]pyridin-6-yl)-6-(4-piperazin-1-yl-1-piperidyl)pyridin-2-amine;
6-[4-[(3R,4R)-3-amino-4-methoxy-pyrrolidin-1-yl]-1-piperidyl]-N,N-dimethyl-3-(8-methylimidazo[1,2-a]pyridin-6-yl)pyridin-2-amine;
6-[2-methoxy-6-(4-piperazin-1-yl-1-piperidyl)-3-pyridyl]-8-methyl-imidazo[1,2-a]pyridine;
(3R,4R)-1-[1-[4-ethyl-5-(8-methylimidazo[1,2-a]pyridin-6-yl)pyrimidin-2-yl]-4-piperidyl]-4-methoxy-pyrrolidin-3-amine;
6-[4-ethyl-2-(4-piperazin-1-yl-1-piperidyl)pyrimidin-5-yl]-8-methyl-imidazo[1,2-a]pyridine; and
2-[1-[4-ethyl-5-(8-methylimidazo[1,2-a]pyridin-6-yl)pyrimidin-2-yl]-4-piperidyl]-5-oxa-2,8-diazaspiro[3.5]nonane;
   or a pharmaceutically acceptable salt thereof.

SYNTHESIS

The compounds of the present invention can be prepared by any conventional means. Suitable processes for synthesizing these compounds as well as their starting materials are provided in the schemes below and in the examples. All substituents, in particular, R¹, R², R³, R⁴, R⁵ and A are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

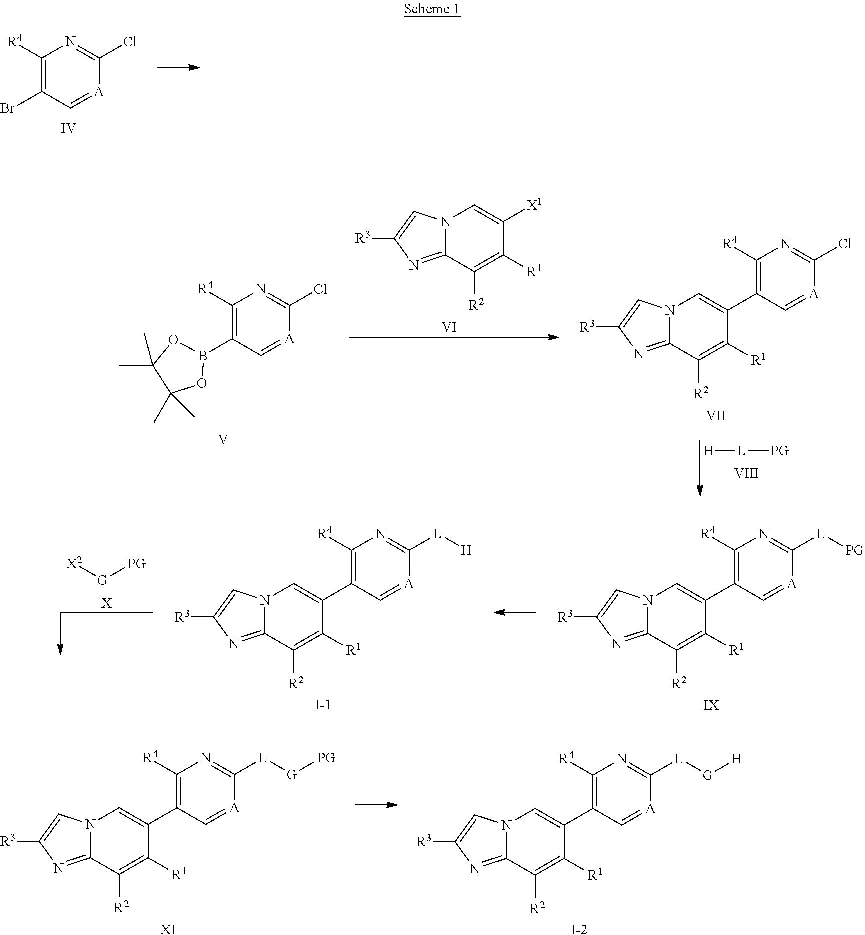

Wherein X¹ and X² are halogen; A is N or CR⁶; PG is a protecting group, such as Boc; L is unsubstituted or substituted group selected from piperazinyl, piperidinyl, 1,3,4,6,7,8,9,9a-octahydropyrazino[1,2-a]pyrazinyl, 3,9-diazaspiro[5.5]undecanyl, 4-oxa-1,9-diazaspiro[5.5]undecanyl, 5-oxa-2,8-diazaspiro[3.5]nonanyl, azetidinyl and pyrrolidinyl; G is 5,6,7,8-tetrahydropyrido[3,4-d]pyrimidinyl, amino(C₁₋₆alkoxy)pyrrolidinyl, C₁₋₆alkylpiperazinyl and piperazinyl.

Compound of formula (IV) is treated with bis(pinacolato)diboron in the presence of a suitable base, such as KOAc, and a suitable palladium catalyst, such as PdCl₂(DPPF)-CH₂Cl₂ adduct, to afford compound of formula (V). Suzuki-coupling reaction between compound of formula (V) and compound of formula (VI) with a suitable catalyst, such as PdCl₂(DPPF)-CH₂Cl₂ adduct, and a suitable base, such as K₂CO₃, affords compound of formula (VII). Compound of formula (VII) undergoes Buchwald-Hartwig amination with compound (VIII) in the presence of a catalyst, such as RuPhos Pd G2, and a suitable base, such as Cs₂CO₃ or t-BuONa to afford compound of formula (IX). Deprotection of compound of formula (IX) under acidic condition, such as TFA, affords compound of (I-1). Coupling of compound of formula (I-1) with compound of formula (X) under Buchwald-Hartwig amination conditions with a catalyst, such as RuPhos Pd G2, and a suitable base, such as Cs₂CO₃ or t-BuONa, affords compound of formula (XI). Deprotection of compound of formula (XI) under acidic condition, such as TFA, affords compound of formula (I-2).

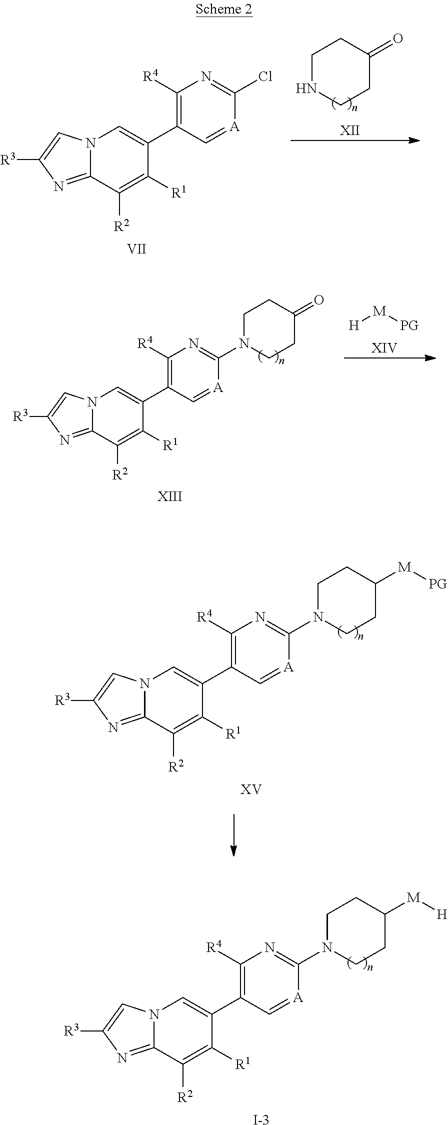

Wherein A is N or CR⁶; PG is a protecting group, such as Boc; n is 0, 1 or 2; M is amino(C₁₋₆alkoxy)pyrrolidinyl, C₁₋₆alkylpiperazinyl or piperazinyl.

Compound of formula (VII) undergoes Buchwald-Hartwig amination with compound (XII) in the presence of a catalyst, such as RuPhos Pd G2, and a suitable base, such as Cs₂CO₃ or t-BuONa to afford compound of formula (XIII) Treating compound of formula XIII with compound of formula XIV with a reductant, such as NaBH(OAc)₃ affords compound of formula XV. Deprotection of compound of formula (XV) under acidic condition, such as TFA affords compound of I-3.

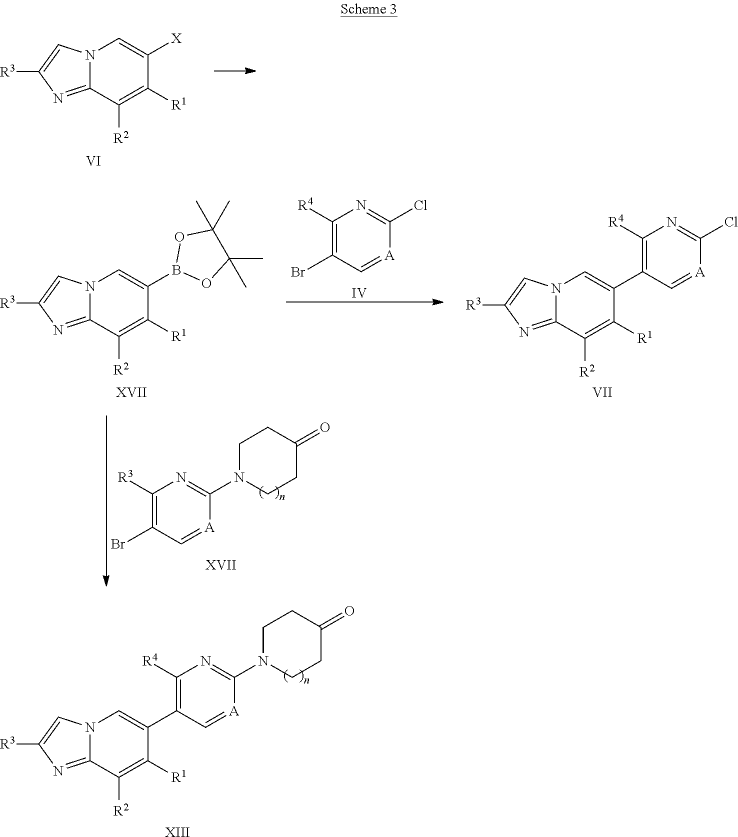

Wherein X is halogen, A is N or CR⁶; n is 0, 1 or 2.

Compound of formula VII and XIII can also be obtained via scheme 3.

Treating compound of formula (VI) with bis(pinacolato)diboron in the presence of a suitable base, such as KOAc, and a suitable palladium catalyst, such as PdCl₂(DPPF)-CH₂Cl₂ adduct, affords compound of formula (XVII). Coupling compound of formula (XVII) with compound of formula (IV) under Suzuki-coupling condition with a suitable catalyst, such as PdCl₂(DPPF)-CH₂Cl₂ adduct, and a suitable base, such as K₂CO₃, affords compound of formula (VII).

Compounds of this invention can be obtained as mixtures of diastereomers or enantiomers, which can be separated by methods well known in the art, e.g. (chiral) HPLC or SFC.

This invention also relates to a process for the preparation of a compound of formula (I) comprising any of the following steps:
a) Deprotection of compound of formula (IX),

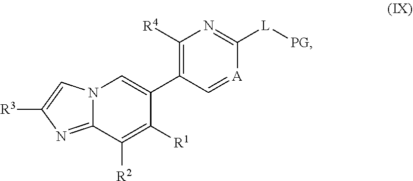

with an acid to afford compound of formula (I-1),

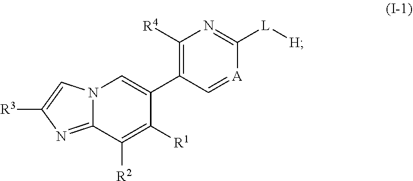

b) Deprotection of compound of formula (XI),

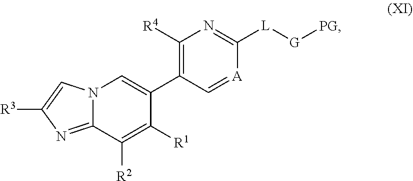

with an acid to afford compound of formula (I-2),

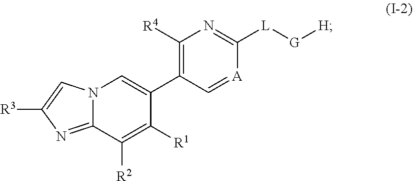

c) Deprotection of compound of formula (XV),

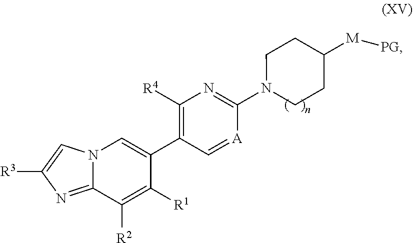

with an acid to afford compound of formula (I-3),

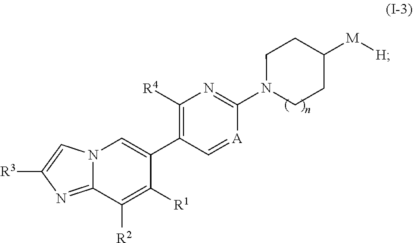

wherein

L is unsubstituted or substituted group selected from piperazinyl, piperidinyl, 1,3,4,6,7,8,9,9a-octahydropyrazino[1,2-a]pyrazinyl, 3,9-diazaspiro[5.5]undecanyl, 4-oxa-1,9-diazaspiro[5.5]undecanyl, 5-oxa-2,8-diazaspiro[3.5]nonanyl, azetidinyl and pyrrolidinyl;

G is 5,6,7,8-tetrahydropyrido[3,4-d]pyrimidinyl, amino(C₁₋₆alkoxy)pyrrolidinyl, C₁₋₆alkylpiperazinyl and piperazinyl;

M is amino(C₁₋₆alkoxy)pyrrolidinyl, C₁₋₆alkylpiperazinyl or piperazinyl;

n is 0, 1 or 2;

in step a), b) and c) the acid can be, for example, TFA.

A compound of formula (I) when manufactured according to the above process is also an object of the invention.

Indications and Methods of Treatment

The present invention provides compounds that can be used as TLR7 and/or TLR8 and/or TLR9 antagonist, which inhibits pathway activation through TLR7 and/or TLR8 and/or TLR9 as well as respective downstream biological events including, but not limited to, innate and adaptive immune responses mediated through the production of all types of cytokines and all forms of auto-antibodies. Accordingly, the compounds of the invention are useful for blocking TLR7 and/or TLR8 and/or TLR9 in all types of cells that express such receptor(s) including, but not limited to, plasmacytoid dendritic cell, B cell, T cell, macrophage, monocyte, neutrophil, keratinocyte, epithelial cell. As such, the compounds can be used as a therapeutic or prophylactic agent for systemic lupus erythematosus and lupus nephritis.

The present invention provides methods for treatment or prophylaxis of systemic lupus erythematosus and lupus nephritis in a patient in need thereof.

Another embodiment includes a method of treating or preventing systemic lupus erythematosus and lupus nephritis in a mammal in need of such treatment, wherein the method comprises administering to said mammal a therapeutically effective amount of a compound of formula (I), a stereoisomer, tautomer, prodrug or pharmaceutically acceptable salt thereof.

### EXAMPLES

The invention will be more fully understood by reference to the following examples. They should not, however, be construed as limiting the scope of the invention.

### ABBREVIATIONS

The invention will be more fully understood by reference to the following examples. They should not, however, be construed as limiting the scope of the invention.

Abbreviations used herein are as follows:
ACN: acetonitrile
Boc₂O: di-tert butyl dicarbonate
NaBH(OAc)₃: sodium triacetoxyborohydride
DCM: dichloromethane
DIPEA: N,N-diisopropylethylamine
DMF: N,N-dimethylformamide
EtOAc or EA: ethyl acetate
FA: formic acid
IC₅₀: half inhibition concentration
LCMS liquid chromatography-mass spectrometry
MS: mass spectrometry
PE: petroleum ether
prep-HPLC: preparative high performance liquid chromatography
PdCl₂(dppf)-CH₂Cl₂: [1,1′-Bis(diphenylphosphino)ferrocene]dichloropalladium(II)
RuPhos Pd G2: chloro(2-dicyclohexylphosphino-2′,6′-diisopropoxy-1,1′-biphenyl)[2-(2′-amino -1,1′-biphenyl)]palladium(II) 2nd generation
SFC: supercritical fluid chromatography
TEA: trimethylamine
TFA: trifluoroacetic acid
v/v: volume ratio
PBS: Phosphate Buffered Saline

General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.
Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### Preparative Examples

The following examples are intended to illustrate the meaning of the present invention but should by no means represent a limitation within the meaning of the present invention:

### Intermediate A1

### 6-bromo-7,8-dimethylimidazo[1,2-a]pyridine

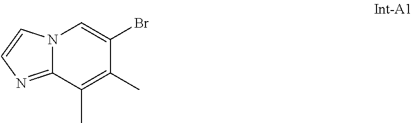

To a solution of 5-bromo-3,4-dimethylpyridin-2-amine (207 mg, 1.03 mmol, CAS No. 374537-97-0, vendor: Bide Pharmatech, catalog BD70340), 2-bromo-1,1-diethoxyethane (406 mg, 2.06 mmol, CAS No. 2032-35-1, vendor: Bide Pharmatech, catalog PBN20120554) in 2-propanol (2 mL) was added pyridine 4-methylbenzenesulfonate (25.9 mg, 103 μmol, CAS No. 24057-28-1, vendor: Bide Pharmatech, catalog BD148963-100g) and the mixture was then stirred at 130° C. for 12 hours. The mixture was then cooled to room temperature and the resulting suspension was filtered. The filtered cake was collected and dried in vacuo to afford 6-bromo-7,8-dimethylimidazo[1,2-a]pyridine (200 mg, 86.3% yield) as a light brown solid. MS calc'd 225 (M+H⁺), measured 225 (M+H⁺).

### Intermediate A2

### 6-bromo-2,8-dimethyl-imidazo[1,2-a]pyridine

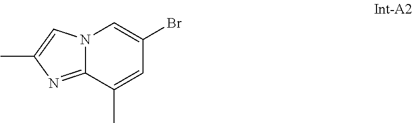

To a solution of 5-bromo-3-methyl-pyridin-2-amine (935 mg, 5.0 mmol, CAS No. vendor: Bide Pharmatech, catalog), chloroacetone (0.44 mL, 5.5 mmol, CAS No., vendor: Bide Pharmatech, catalog) in DMF (5 mL) was stirred at 90° C. for 2 hours. The mixture was then cooled to room temperature and the resulting suspension was partitioned between DCM (20 mL) and saturated NaHCO₃ solution (30 mL). The organic layer was separated out, washed with brine, dried over Na₂SO₄ and then concentrated in vacuo to give 6-bromo-2,8-dimethyl-imidazo[1,2-a]pyridine (300 mg, 26.7% yield) as a light brown solid. MS calc'd 225 (M+H⁺), measured 225 (M+H⁺).

### Intermediate A3

### 6-bromo-8-methoxy-imidazo[1,2-a]pyridine

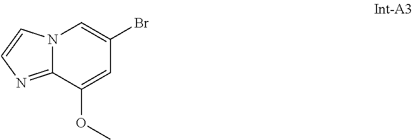

Int-A3 was prepared in analogy to the preparation of Int-A1 by using 5-bromo-3-methoxy-pyridin-2-amine (CAS No. 42409-58-5, vendor: Bide Pharmatech, catalog BD196595) instead of 5-bromo-3,4-dimethylpyridin-2-amine. Int-A3 (320 mg) was obtained as a light yellow solid. MS: calc'd 227 (M+H⁺), measured 227 (M+H⁺).

### Intermediate B1

### 3-bromo-6-chloro-2-isopropylpyridine

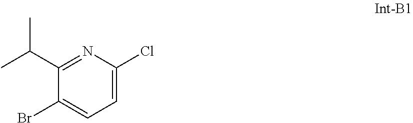

Compound Int-B1 was prepared according to the following scheme:

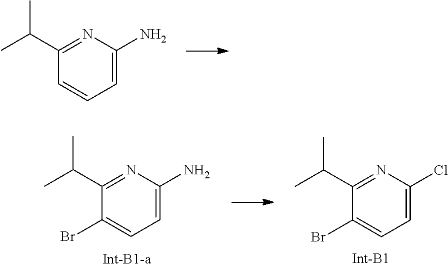

### Step 1: preparation of 5-bromo-6-isopropylpyridin-2-amine

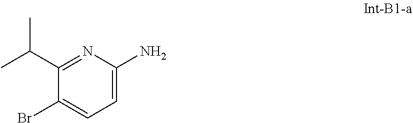

To a solution of 6-isopropylpyridin-2-amine (3.2 g, 23.5 mmol, CAS NO. 78177-12-5, vendor: Accela ChemBio Inc, catalog SY006009-5g) in MeOH (40 mL) was added N-bromosuccinimide (4.18 g, 23.5 mmol, CAS NO. 128-08-5, vendor: Accela ChemBio Inc, catalog SY001733) at 0° C. The mixture was then warmed to room temperature and stirred at room temperature for 16 hours. After the reaction was completed, the mixture was quenched with water (60 mL), extracted with EA (50 mL) twice. The combined organic layer was washed with brine, dried over Na₂SO₄ and concentrated in vacuo. The residue was then purified by flash chromatography eluting with a gradient of EA/PE (0% to 50%) to afford 5-bromo-6-isopropylpyridin-2-amine (3.8 g, 75.2% yield) as yellow oil. MS: calc'd 215 (M+H⁺), measured 215 (M+H⁺).

### Step 2: preparation of 3-bromo-6-chloro-2-isopropylpyridine

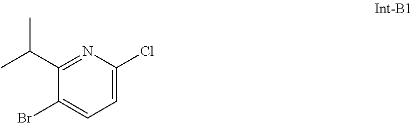

A suspension of 5-bromo-6-isopropylpyridin-2-amine (3.6 g, 16.7 mmol), copper (I) chloride (828 mg, 8.37 mmol, CAS NO. 7758-89-6, vendor: Bide Pharmatech, catalog BD122484) and copper (II) chloride (3.38 g, 25.1 mmol, CAS NO. 10125-13-0, vendor: Accela ChemBio Inc, catalog SY009935) in 1,2-dichloroethane (40 mL) was stirred at 0° C. for 10 minutes. Then to the resulting suspension was added tert-butyl nitrite (3.45 g, 3.98 mL, 33.5 mmol, CAS NO. 540-80-7, vendor: TCI Shanghai, catalog N0357). The mixture was stirred at 0° C. for another 1 hour under N₂ atmosphere and then the mixture was stirred at 70° C. for 48 hours. After the reaction was completed, the mixture was cooled to room temperature, quenched with water (30 mL), extracted with DCM (30 mL) three times. The combined organic layer was washed with brine, dried over MgSO₄, filtered and concentrated in vacuo. The residue was purified by flash chromatography eluting with a gradient of EA/PE (0% to 20%) to afford 3-bromo-6-chloro-2-isopropylpyridine (2.475 g, 63.1% yield) as yellow oil. ¹H NMR (400 MHz, METHANOL-d₄) δ 7.90 (d, J=8.3 Hz, 1H), 7.14 (d, J=8.3 Hz, 1H), 3.53 (td, J=6.7, 13.5 Hz, 1H), 1.24 (d, J=6.7 Hz, 6H). MS calc'd 234 (M+H⁺), measured 234 (M+H⁺).

### Intermediate B2

### 6-chloro-2-isopropyl-3-(4,4,5,5-tetramethyl-1,3,2-dioxaborolan-2-yl)pyridine

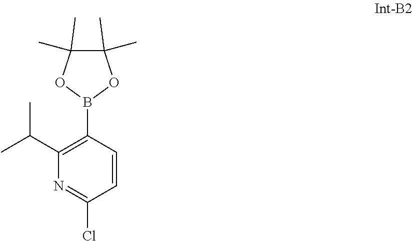 [SMILES: CC(C)c1nc(Cl)ccc1B1OC(C)(C)C(C)(C)O1]

To a mixture of 3-bromo-6-chloro-2-isopropylpyridine (2.475 g, 10.6 mmol), bis(pinacolato)diboron (3.22 g, 12.7 mmol, CAS No. 73183-34-3, vendor: Accela ChemBio Inc, catalog SY001323), potassium acetate (2.59 g, 26.4 mmol) in dioxane (18 mL) was added PdCl₂(dppf)-CH₂Cl₂ adduct (862 mg, 1.06 mmol) and the mixture was then stirred at 90° C. under N₂ atmosphere for 2 hours. After cooling, the mixture was filtered and the solid was washed with EA (20 mL) twice. The combined organic layer was washed with brine, dried over Na₂SO₄, filtered and concentrated in vacuo. The residue was purified by flash chromatography eluting with a gradient of EA/PE (0% to 40%) to afford 6-chloro-2-isopropyl-3-(4,4,5,5-tetramethyl-1,3,2-dioxaborolan-2-yl)pyridine (2.8 g, 94.2% yield) as a light yellow solid. MS calc'd 282 (M+H⁺), measured 282 (M+H⁺).

### Intermediate B3

### 3-bromo-6-chloro-2-ethyl-pyridine

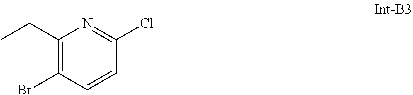

Compound Int-B3 was prepared in analogy to the preparation of compound Int-B1 by using 6-ethylpyridin-2-amine instead of 6-isopropylpyridin-2-amine in step 1. MS calc'd 220 (M+H⁺), measured 220 (M+H⁺).

### Intermediate B4

### 6-chloro-2-ethyl-3-(4,4,5,5-tetramethyl-1,3,2-dioxaborolan-2-yl)pyridine

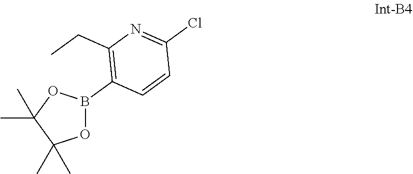 [SMILES: CCc1nc(Cl)ccc1B1OC(C)(C)C(C)(C)O1]

Compound Int-B4 was prepared in analogy to the preparation of compound Int-B2 by using 3-bromo-6-chloro-2-ethyl-pyridine instead of 3-bromo-6-chloro-2-isopropyl-pyridine. MS calc'd 268 (M+H⁺), measured 268 (M+H⁺).

### Intermediate B5

### 3-bromo-6-chloro-2-(difluoromethyl)pyridine

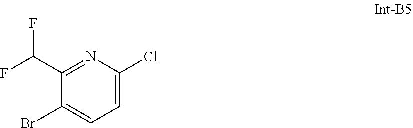 [SMILES: FC(F)c1nc(Cl)ccc1Br]

3-bromo-6-chloropicolinaldehyde (1.5 g, 6.8 mmol) was dissolved in DCM (20 mL), and the solution was cooled to −78° C., followed by addition of (diethylamino)sulfur trifluoride (4.39 g, 3.6 mL, 27.2 mmol, CAS No. 38078-09-0, vendor: PharmaBlock Sciences (Nanjing), Inc., catalog PBLY8231), the mixture was stirred for 30 min at −78° C., and then stirred at room temperature for 10 hours. After the reaction was completed, the mixture was quenched with 2M K₂CO₃ solution (30 mL) and the resulting mixture was extracted with DCM (50 mL) twice. The combined organic layer was concentrated in vacuo, the residue was then purified by flash column eluting with a gradient of EA/PE (0% to 50%) to give 3-bromo-6-chloro-2-(difluoromethyl)pyridine (1.5 g, 90.9% yield) as a little yellow solid. MS calc'd 242 (M+H⁺), measured 242 (M+H⁺).

### Intermediate B6

### 5-bromo-2-chloro-3-ethyl-pyridine

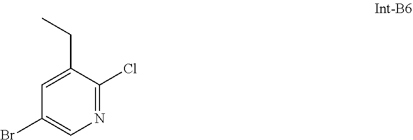

Compound Int-B6 was prepared in analogy to the preparation of compound Int-B1 by using 3-ethylpyridin-2-amine (CAS NO. 78177-12-5, vendor: Accela ChemBio Inc, catalog SY006009) instead of 6-isopropylpyridin-2-amine in step 1. 5-bromo-2-chloro-3-ethyl-pyridine (500 mg) was obtained as a yellow liquid. MS calc'd 221 (M+H⁺), measured 221 (M+H⁺).

### Intermediate B7

### 2-chloro-3-ethyl-5-(4,4,5,5-tetramethyl-1,3,2-dioxaborolan-2-yl)pyridine

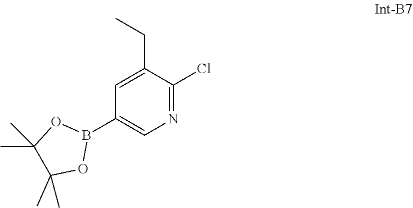 [SMILES: CCc1cc(B2OC(C)(C)C(C)(C)O2)cnc1Cl]

Compound Int-B7 was prepared in analogy to the preparation of compound Int-B2 by using 5-bromo-2-chloro-3-ethyl-pyridine instead of 3-bromo-6-chloro-2-isopropyl-pyridine. 2-chloro-3-ethyl-5-(3,3,4,4-tetramethyl-1λ³,2,5-bromadioxolan-1-yl)pyridine (190 mg) was obtained as a yellow oil. MS calc'd 268 (M+H⁺), measured 268 (M+H⁺).

### Example 1

### 1-[6-isopropyl-5-(8-methylimidazo[1,2-a]pyridin-6-yl)-2-pyridyl]piperidin-4-amine

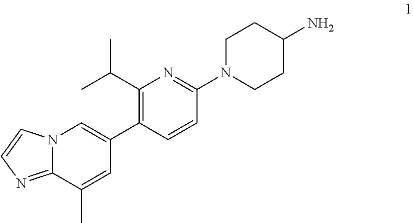 [SMILES: Cc1cc(-c2ccc(N3CCC(N)CC3)nc2C(C)C)cn2ccnc12]

The title compound was prepared according to the following scheme:

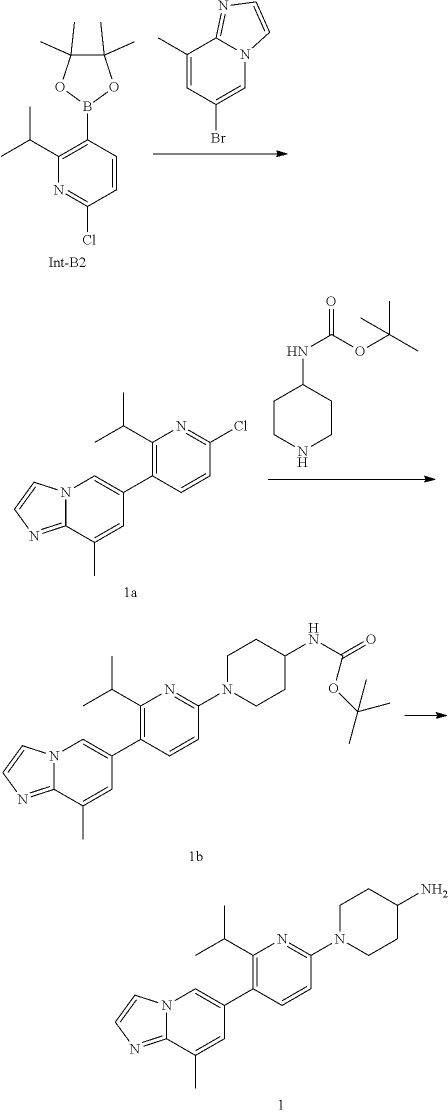

### Step 1: preparation of 6-(6-chloro-2-isopropylpyridin-3-yl)-8-methylimidazo[1,2-a]pyridine

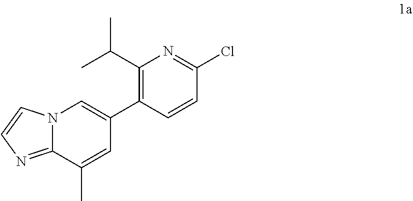 [SMILES: Cc1cc(-c2ccc(Cl)nc2C(C)C)cn2ccnc12]

A mixture of 6-chloro-2-isopropyl-3-(4,4,5,5-tetramethyl-1,3,2-dioxaborolan-2-yl)pyridine (800 mg, 2.84 mmol), 6-bromo-8-methylimidazo[1,2-a]pyridine (600 mg, 2.84 mmol, CAS No. 217435-65-9, vendor: Accela ChemBio Inc, catalog SY039846) and PddCl₂(DPPF) (232 mg, 284 μmol) in mixed solvent of dioxane (20 mL) and Na₂CO₃ (2M, 6 mL) was charged with N₂, and the mixture was stirred at 90° C. for 1 hour. After the reaction was completed, the mixture was then concentrated in vacuo and the residue was purified by flash chromatography eluting with a gradient of EA/PE (0% to 100%) to afford 6-(6-chloro-2-isopropylpyridin-3-yl)-8-methylimidazo[1,2-a]pyridine (600 mg, 66.5% yield) as light brown oil. MS: calc'd 286 (M+H⁺), measured 286 (M+H⁺).

### Step 2: preparation of tert-butyl (1-(6-isopropyl-5-(8-methylimidazo[1,2-a]pyridin-6-yl)pyridin-2-yl)piperidin-4-yl)carbamate

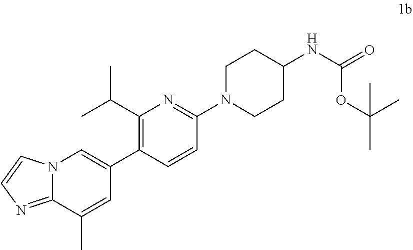 [SMILES: Cc1cc(-c2ccc(N3CCC(NC(=O)OC(C)(C)C)CC3)nc2C(C)C)cn2ccnc12]

To a solution of 6-(6-chloro-2-isopropylpyridin-3-yl)-8-methylimidazo[1,2-a]pyridine (50 mg, 175 μmol), tert-butyl piperidin-4-ylcarbamate (42 mg, 210 μmol) and Cs₂CO₃ (171 mg, 525 μmol) in dioxane (2 mL) was added RuPhos Pd G2 (13.6 mg, 17.5 μmol) and the reaction mixture was stirred at 105° C. under N₂ for 16 hours. After the reaction was completed, the mixture was then concentrated in vacuo and the residue was purified by flash chromatography eluting with a gradient of MeOH/DCM (0% to 15%) to afford tert-butyl (1-(6-isopropyl-5-(8-methylimidazo[1,2-a]pyridin-6-yl)pyridin-2-yl)piperidin-4-yl)carbamate (47 mg, 59.7% yield) as a light yellow solid. MS: calc'd 450 (M+H⁺), measured 450 (M+H⁺).

### Step 3: preparation of 1-[6-isopropyl-5-(8-methylimidazo[1,2-a]pyridin-6-yl)-2-pyridyl]piperidin-4-amine

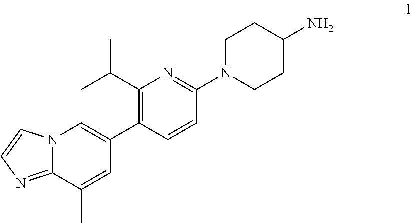 [SMILES: Cc1cc(-c2ccc(N3CCC(N)CC3)nc2C(C)C)cn2ccnc12]

To a solution of tert-butyl (1-(6-isopropyl-5-(8-methylimidazo[1,2-a]pyridin-6-yl)pyridin-2-yl)piperidin-4-yl)carbamate (47 mg) in DCM (2 mL) cooled at 0° C. was added TFA (1 mL). The reaction mixture was stirred at room temperature for 2 hours. After the reaction was completed, the mixture was then concentrated in vacuo and the residue was purified by prep-HPLC to afford 1-[6-isopropyl-5-(8-methylimidazo[1,2-a]pyridin-6-yl)-2-pyridyl]piperidin-4-amine (34 mg, 98% yield) as a light yellow solid. MS: calc'd 350 (M+H⁺), measured 350 (M+H⁺). ¹H NMR (400 MHz, METHANOL-d₄) δ 8.57 (s, 1H), 8.23 (d, J 2.1 Hz, 1H), 8.08 (d, J=2.1 Hz, 1H), 7.69 (s, 1H), 7.46 (d, J=8.7 Hz, 1H), 6.80 (d, J=8.7 Hz, 1H), 4.60 (br d, J 13.7 Hz, 2H), 3.46-3.36 (m, 1H), 3.09-2.96 (m, 3H), 2.70 (s, 3H), 2.14-2.04 (m, 2H), 1.64 (dq, J=4.2, 12.2 Hz, 2H), 1.21 (d, J=6.7 Hz, 6H).

### Example 2

### 6-[2-isopropyl-6-(4-piperazin-1-yl-1-piperidyl)-3-pyridyl]-8-methyl-imidazo[1,2-a]pyridine

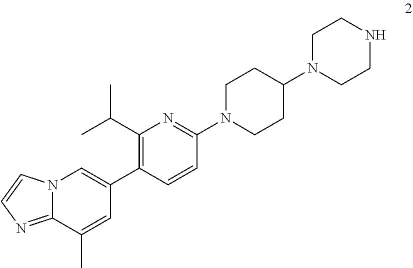 [SMILES: Cc1cc(-c2ccc(N3CCC(N4CCNCC4)CC3)nc2C(C)C)cn2ccnc12]

The title compound was prepared in analogy to the preparation of Example 1 by using tert-butyl 4-(piperidin-4-yl)piperazine-1-carboxylate (CAS No. 205059-24-1, vendor: Bide Pharmatech, catalog BD57121) instead of tert-butyl piperidin-4-ylcarbamate in step 2.

Example 2 (40 mg) was obtained as a light yellow solid. ¹H NMR (400 MHz, METHANOL-d₄) δ 8.58 (s, 1H), 8.24 (d, J 2.1 Hz, 1H), 8.08 (d, J 2.1 Hz, 1H), 7.69 (s, 1H), 7.49 (d, J 8.7 Hz, 1H), 6.83 (d, J 8.8 Hz, 1H), 4.70 (br d, J 13.7 Hz, 2H), 3.74-3.58 (m, 9H), 3.09-2.96 (m, 3H), 2.70 (s, 3H), 2.25 (br d, J=11.0 Hz, 2H), 1.80 (dq, J=4.0, 12.1 Hz, 2H), 1.22 (d, J=6.7 Hz, 6H). MS: calc'd 419 (M+H⁺), measured 419 (M+H⁺).

### Example 3

### 6-[2-isopropyl-6-(4-piperazin-1-yl-1-piperidyl)-3-pyridyl]-2,8-dimethyl-imidazo[1,2-a]pyridine

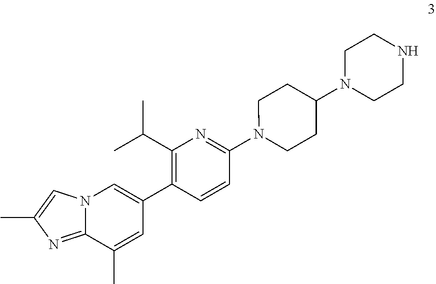 [SMILES: Cc1cn2cc(-c3ccc(N4CCC(N5CCNCC5)CC4)nc3C(C)C)cc(C)c2n1]

### Step 1: preparation of 6-(6-chloro-2-isopropyl-3-pyridyl)-2,8-dimethyl-imidazo[1,2-a]pyridine

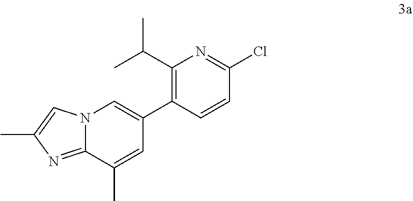 [SMILES: Cc1cn2cc(-c3ccc(Cl)nc3C(C)C)cc(C)c2n1]

Compound 3a was prepared in analogy to the preparation of compound 1a by using 6-bromo-2,8-dimethylimidazo[1,2-a]pyridine (Int-A2) instead of 6-bromo-8-methylimidazo[1,2-a]pyridine in Step 1. MS: calc'd 300 (M+H⁺), measured 300 (M+H⁺).

### Step 2: preparation of 6-[2-isopropyl-6-(4-piperazin-1-yl-1-piperidyl)-3-pyridyl]-2,8-dimethyl-imidazo[1,2-a]pyridine

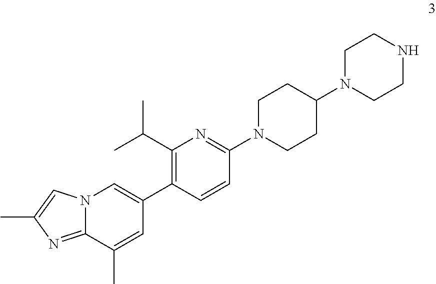 [SMILES: Cc1cn2cc(-c3ccc(N4CCC(N5CCNCC5)CC4)nc3C(C)C)cc(C)c2n1]

The title compound was prepared in analogy to the preparation of Example 1 by using 6-(6-chloro-2-isopropyl-3-pyridyl)-2,8-dimethyl-imidazo[1,2-a]pyridine and tert-butyl 4-(piperidin-4-yl)piperazine-1-carboxylate instead of 6-(6-chloro-2-isopropylpyridin-3-yl)-8-methylimidazo[1,2-a]pyridine and tert-butyl piperidin-4-ylcarbamate in step 2.

Example 3 (7 mg) was obtained as a white solid. ¹H NMR (400 MHz, METHANOL-d₄) δ 8.47-8.44 (m, 1H), 7.94 (d, J=1.0 Hz, 1H), 7.62 (t, J=1.2 Hz, 1H), 7.44 (d, J=8.7 Hz, 1H), 6.79 (d, J=8.8 Hz, 1H), 4.69 (br d, J=13.6 Hz, 2H), 3.61-3.47 (m, 9H), 3.06-2.91 (m, 3H), 2.66 (s, 3H), 2.58 (d, J=1.0 Hz, 3H), 2.24-2.16 (m, 2H), 1.75 (dq, J=4.1, 12.1 Hz, 2H), 1.20 (d, J=6.6 Hz, 6H). MS: calc'd 432 (M+H⁺), measured 432 (M+H⁺).

### Example 4

### 1-[5-(2,8-dimethylimidazo[1,2-a]pyridin-6-yl)-6-isopropyl-2-pyridyl]piperidin-4-amine

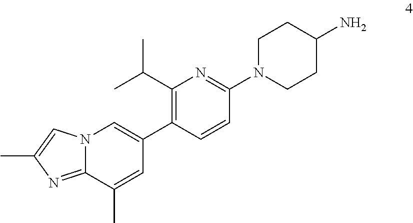 [SMILES: Cc1cn2cc(-c3ccc(N4CCC(N)CC4)nc3C(C)C)cc(C)c2n1]

The title compound was prepared in analogy to the preparation of Example 1 by using 6-(6-chloro-2-isopropyl-3-pyridyl)-2,8-dimethyl-imidazo[1,2-a]pyridine instead of 6-(6-chloro-2-isopropylpyridin-3-yl)-8-methylimidazo[1,2-a]pyridine in step 2.

Example 4 (12 mg) was obtained as a light yellow solid. ¹H NMR (400 MHz, METHANOL-d₄) δ 8.46 (s, 1H), 7.94 (d, J=1.1 Hz, 1H), 7.61 (s, 1H), 7.43 (d, J=8.7 Hz, 1H), 6.78 (d, J=8.7 Hz, 1H), 4.59 (br d, J=13.6 Hz, 2H), 3.46-3.35 (m, 1H), 3.06-2.95 (m, 3H), 2.66 (s, 3H), 2.58 (d, J=0.9 Hz, 3H), 2.07 (br s, 2H), 1.63 (dq, J=4.2, 12.2 Hz, 2H), 1.20 (d, J=6.6 Hz, 6H). MS: calc'd 364 (M+H⁺), measured 364 (M+H⁺).

### Example 5

### [(2S)-1-[5-(2,8-dimethylimidazo[1,2-a]pyridin-6-yl)-6-isopropyl-2-pyridyl]piperazin-2-yl]methanol

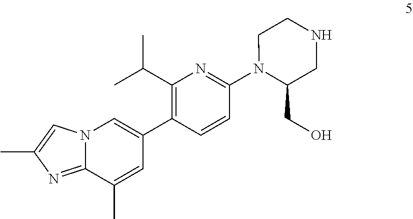 [SMILES: Cc1cn2cc(-c3ccc(N4CCNC[C@H]4CO)nc3C(C)C)cc(C)c2n1]

The title compound was prepared in analogy to the preparation of Example 1 by using 6-(6-chloro-2-isopropyl-3-pyridyl)-2,8-dimethyl-imidazo[1,2-a]pyridine and tert-butyl (3S)-3-(hydroxymethyl)piperazine-1-carboxylate (CAS No. 314741-40-7, vendor: PharmaBlock (Nanjing) R&D Co. Ltd, catalog PBN20121940) instead of 6-(6-chloro-2-isopropylpyridin-3-yl)-8-methylimidazo[1,2-a]pyridine and tert-butyl piperidin-4-ylcarbamate in step 2.

Example 5 (12 mg) was obtained as a light yellow solid. ¹H NMR (400 MHz, METHANOL-d₄) δ 8.49 (s, 1H), 7.97-7.93 (m, 1H), 7.64 (s, 1H), 7.50 (d, J=8.7 Hz, 1H), 6.79 (d, J=8.8 Hz, 1H), 4.46 (br dd, J=2.5, 14.0 Hz, 1H), 3.96 (dq, J=4.8, 11.0 Hz, 2H), 3.77 (br d, J=13.0 Hz, 1H), 3.66-3.50 (m, 2H), 3.41-3.31 (m, 2H), 3.23 (dt, J=4.0, 12.5 Hz, 1H), 3.04 (td, J=6.6, 13.3 Hz, 1H), 2.66 (s, 3H), 2.59 (s, 3H), 1.20 (dd, J=2.9, 6.7 Hz, 6H). MS: calc'd 493 (M+H⁺), measured 493 (M+H⁺).

### Example 6

### 9-[5-(2,8-dimethylimidazo[1,2-a]pyridin-6-yl)-6-isopropyl-2-pyridyl]-4-oxa-1,9-diazaspiro[5.5]undecane

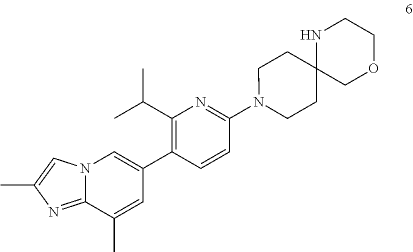 [SMILES: Cc1cn2cc(-c3ccc(N4CCC5(CC4)COCCN5)nc3C(C)C)cc(C)c2n1]

The title compound was prepared in analogy to the preparation of Example 1 by using 6-(6-chloro-2-isopropyl-3-pyridyl)-2,8-dimethyl-imidazo[1,2-a]pyridine and tert-butyl 1-oxa-4,9-diazaspiro[5.5]undecane-9-carboxylate (CAS No. 1368040-61-2, vendor: Bide Pharmatech, catalog BD00848365) instead of 6-(6-chloro-2-isopropylpyridin-3-yl)-8-methylimidazo[1,2-a]pyridine and tert-butyl piperidin-4-ylcarbamate in step 2.

Example 6 (11 mg) was obtained as a light yellow solid. ¹H NMR (400 MHz, METHANOL-d₄) δ 8.46 (s, 1H), 7.94 (d, J=1.0 Hz, 1H), 7.62 (s, 1H), 7.43 (d, J=8.7 Hz, 1H), 6.77 (d, J=8.8 Hz, 1H), 4.22-4.13 (m, 2H), 4.02-3.93 (m, 2H), 3.37-3.32 (m, 2H), 3.26-3.18 (m, 2H), 3.13 (s, 2H), 3.02 (quin, J=6.7 Hz, 1H), 2.66 (s, 3H), 2.58 (d, J=0.9 Hz, 3H), 2.07 (br d, J=12.8 Hz, 2H), 1.75-1.64 (m, 2H), 1.20 (d, J=6.6 Hz, 6H). MS: calc'd 420 (M+H⁺), measured 420 (M+H⁺).

### Example 7

### 6-[2-isopropyl-6-(4-piperazin-1-yl-1-piperidyl)-3-pyridyl]-7,8-dimethyl-imidazo[1,2-a]pyridine

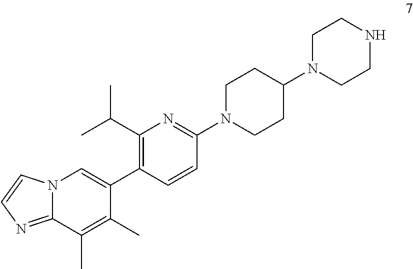 [SMILES: Cc1c(-c2ccc(N3CCC(N4CCNCC4)CC3)nc2C(C)C)cn2ccnc2c1C]

### Step 1: preparation of 6-(6-chloro-2-isopropyl-3-pyridyl)-7,8-dimethyl-imidazo[1,2-a]pyridine

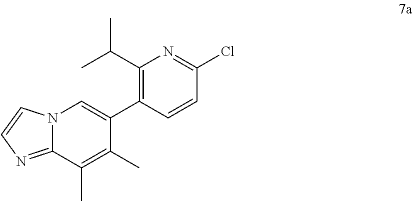 [SMILES: Cc1c(-c2ccc(Cl)nc2C(C)C)cn2ccnc2c1C]

Compound 7a was prepared in analogy to the preparation of compound 1a by using 6-bromo-7,8-dimethylimidazo[1,2-a]pyridine (Int-A1) instead of 6-bromo-8-methylimidazo[1,2-a]pyridine in Step 1. MS: calc'd 300 (M+H⁺), measured 300 (M+H⁺).

### Step 2: preparation 6-[2-isopropyl-6-(4-piperazin-1-yl-1-piperidyl)-3-pyridyl]-7,8-dimethyl-imidazo[1,2-a]pyridine

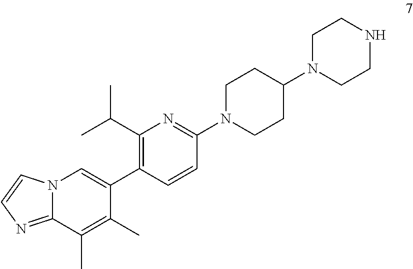 [SMILES: Cc1c(-c2ccc(N3CCC(N4CCNCC4)CC3)nc2C(C)C)cn2ccnc2c1C]

The title compound was prepared in analogy to Example 1 by using 6-(6-chloro-2-isopropyl-3-pyridyl)-7,8-dimethyl-imidazo[1,2-a]pyridine and tert-butyl 4-(piperidin-4-yl)piperazine-1-carboxylate (CAS No. 205059-24-1, vendor: Bide Pharmatech, catalog BD57121-5g) instead of 6-(6-chloro-2-isopropylpyridin-3-yl)-8-methylimidazo[1,2-a]pyridine and tert-butyl piperidin-4-ylcarbamate in step 2.

Example 7 (13 mg) was obtained as a light yellow solid. ¹H NMR (400 MHz, METHANOL-d₄) δ 8.46 (s, 1H), 8.11 (d, J=2.1 Hz, 1H), 8.00 (d, J=2.1 Hz, 1H), 7.33 (d, J 8.7 Hz, 1H), 6.81 (d, J=8.7 Hz, 1H), 4.71 (br t, J=12.8 Hz, 2H), 3.71-3.48 (m, 9H), 3.03-2.93 (m, 2H), 2.69-2.60 (m, 4H), 2.23 (s, 5H), 1.78 (dq, J=3.5, 11.9 Hz, 2H), 1.19 (d, J=6.6 Hz, 3H), 1.11 (d, J=6.6 Hz, 3H). MS: calc'd 433 (M+H⁺), measured 433 (M+H⁺).

### Example 8

### 2-[5-(2,8-dimethylimidazo[1,2-a]pyridin-6-yl)-6-isopropyl-2-pyridyl]-1,3,4,6,7,8,9,9a-octahydropyrazino[1,2-a]pyrazine

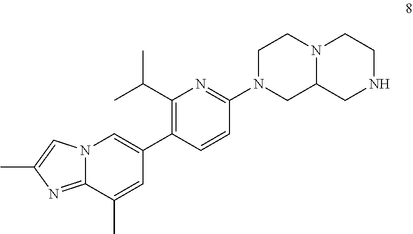 [SMILES: Cc1cn2cc(-c3ccc(N4CCN5CCNCC5C4)nc3C(C)C)cc(C)c2n1]

The title compound was prepared in analogy to the preparation of Example 1 by using 6-(6-chloro-2-isopropyl-3-pyridyl)-2,8-dimethyl-imidazo[1,2-a]pyridine and tert-butyl octahydro-2H-pyrazino[1,2-a]pyrazine-2-carboxylate (CAS No. 1159825-34-9, vendor: Pharmablock, catalog: PB07063) instead of 6-(6-chloro-2-isopropylpyridin-3-yl)-8-methylimidazo[1,2-a]pyridine and tert-butyl piperidin-4-ylcarbamate in step 2.

Example 8 (13.6 mg) was obtained as a white solid. ¹H NMR (400 MHz, CD₃OD) δ 8.06 (s, 1H), 7.57 (s, 1H), 7.37 (d, J=8.4 Hz, 1H), 6.96 (s, 1H), 6.66 (br d, J=8.6 Hz, 1H), 4.43-4.19 (m, 2H), 3.16-2.74 (m, 7H), 2.63-2.48 (m, 5H), 2.43 (s, 3H), 2.41-2.13 (m, 3H), 1.17 (br d, J=6.6 Hz, 6H). MS: calc'd 405 (M+H⁺), measured 405 (M+H⁺).

### Example 9

### 8-methyl-6-[5-methyl-6-[4-(4-methylpiperazin-1-yl)-1-piperidyl]-3-pyridyl]imidazo[1,2-a]pyridine

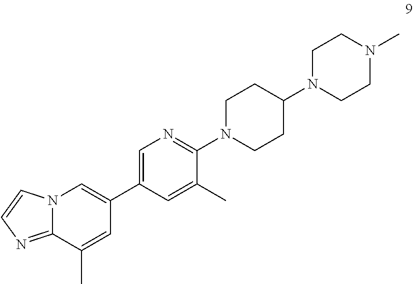 [SMILES: Cc1cc(-c2cc(C)c3nccn3c2)cnc1N1CCC(N2CCN(C)CC2)CC1]

The title compound was prepared according to the following scheme:

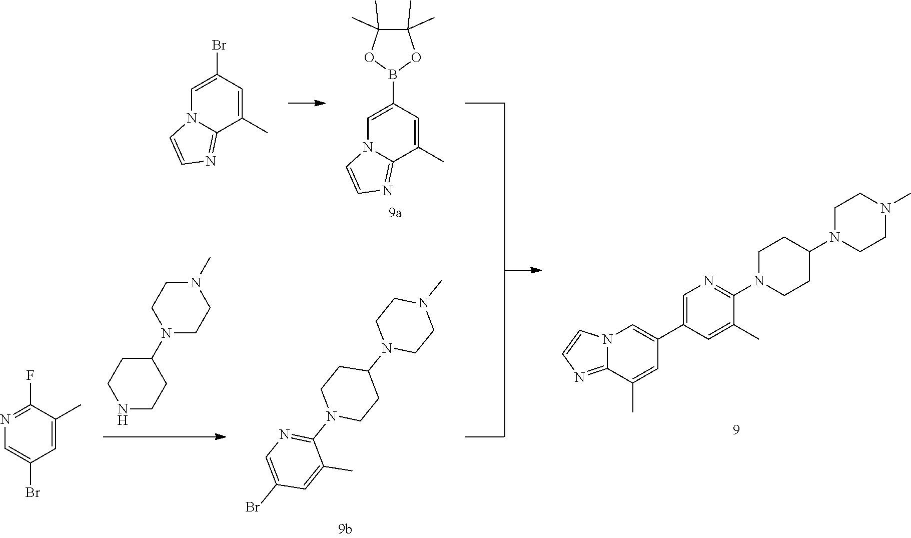

### Step 1: preparation of 8-methyl-6-(4,4,5,5-tetramethyl-1,3,2-dioxaborolan-2-yl)imidazo[1,2-a]pyridine

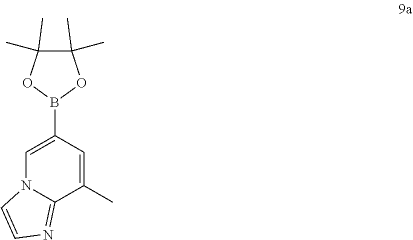 [SMILES: Cc1cc(B2OC(C)(C)C(C)(C)O2)cn2ccnc12]

To a mixture of 6-bromo-8-methylimidazo[1,2-a]pyridine (1 g, 4.74 mmol), bis(pinacolato)diboron (1.44 g, 5.69 mmol, CAS No. 73183-34-3, vendor: Accela ChemBio Inc, catalog SY001323), potassium acetate (1.16 g, 11.8 mmol) in dioxane (8 mL) was added PdCl₂(dppf)-CH₂Cl₂ adduct (387 mg, 0.47 mmol) and the mixture was then stirred at 90° C. under N₂ atmosphere for 2 hours. After cooling, the mixture was filtered off and the solid was washed with EA (20 mL) twice. The combined organic layer was washed with brine, dried over Na₂SO₄, filtered and concentrated to afford the crude 8-methyl-6-(4,4,5,5-tetramethyl-1,3,2-dioxaborolan-2-yl)imidazo[1,2-a]pyridine (1 g, 81.7% yield) as a black solid, which was used in the next step directly without further purification. MS calc'd 259 (M+H⁺), measured 259 (M+H⁺).

### Step 2: preparation of 1-(1-(5-bromo-3-methylpyridin-2-yl)piperidin-4-yl)-4-methylpiperazine

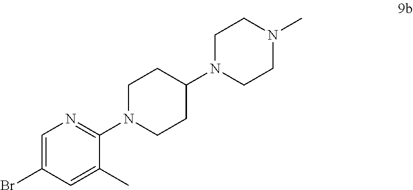 [SMILES: Cc1cc(Br)cnc1N1CCC(N2CCN(C)CC2)CC1]

To a mixture of 5-bromo-2-fluoro-3-methylpyridine (95 mg, 500 μmol), 1-methyl-4-(piperidin-4-yl)piperazine (91.6 mg, 500 μmol) in DMSO (1 mL) was added DIPEA (64.6 mg, 87.3 μL, 500 μmol) and the mixture was then stirred at 130° C. for 16 hours. After cooling down to room temperature, the reaction mixture was then diluted with water (10 mL) and extracted with EA (15 mL) three times. The combined organic layer was washed with brine, dried over Na₂SO₄ and concentrated in vacuo. The residue was purified by flash chromatography eluting with a gradient of EA/PE (0% to 50 to afford 1-(1-(5-bromo-3-methylpyridin-2-yl)piperidin-4-yl)-4-methylpiperazine (36 mg, 20% yield) as colorless oil. MS calc'd 353 (M+H⁺), measured 353 (M+H⁺).

### Step 3: preparation of 8-methyl-6-[5-methyl-6-[4-(4-methylpiperazin-1-yl)-1-piperidyl]-3-pyridyl]imidazo[1,2-a]pyridine

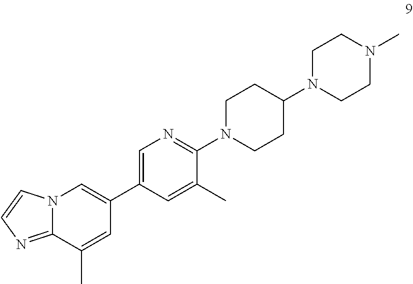 [SMILES: Cc1cc(-c2cc(C)c3nccn3c2)cnc1N1CCC(N2CCN(C)CC2)CC1]

A mixture of 1-(1-(5-bromo-3-methylpyridin-2-yl)piperidin-4-yl)-4-methylpiperazine (36 mg, 102 μmol), 8-methyl-6-(4,4,5,5-tetramethyl-1,3,2-dioxaborolan-2-yl)imidazo[1,2-a]pyridine (50 mg, 194 μmol) and PdCl₂(dppf)-CH₂Cl₂ adduct (8 mg, 10.2 μmol) in a mixed solvent of dioxane (2 mL) and Na₂CO₃ (2M, 1 mL) was charged with N₂ and the mixture was then stirred at 90° C. for 1 hour. After cooling down to room temperature, the mixture was diluted with EtOAC (20 mL), washed with water, brine, dried over Na₂SO₄ and the organic layer was concentrated in vacuo. The residue was purified by Prep-HPLC to afford 8-methyl-6-[5-methyl-6-[4-(4-methylpiperazin-1-yl)-1-piperidyl]-3-pyridyl]imidazo[1,2-a]pyridine (10 mg) as a light yellow solid. ¹H NMR (400 MHz, METHANOL-d₄) δ 8.62 (s, 1H), 8.38-8.34 (m, 1H), 7.92 (d, J=1.2 Hz, 1H), 7.83 (d, J=1.8 Hz, 1H), 7.64 (d, J=1.3 Hz, 1H), 7.47 (s, 1H), 3.61 (br d, J=12.8 Hz, 2H), 3.19-2.95 (m, 8H), 2.92-2.79 (m, 3H), 2.73 (s, 3H), 2.62 (s, 3H), 2.38 (s, 3H), 2.06 (br d, J=11.2 Hz, 2H), 1.79 (br d, J=3.3 Hz, 2H). MS: calc'd 405 (M+H⁺), measured 405 (M+H⁺).

### Example 10

### 6-[2-(difluoromethyl)-6-(4-piperazin-1-yl-1-piperidyl)-3-pyridyl]-8-methyl-imidazo[1,2-a]pyridine

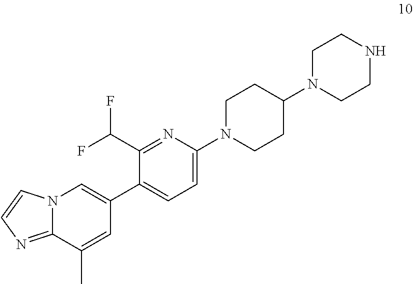 [SMILES: Cc1cc(-c2ccc(N3CCC(N4CCNCC4)CC3)nc2C(F)F)cn2ccnc12]

The title compound was prepared according to the following scheme:

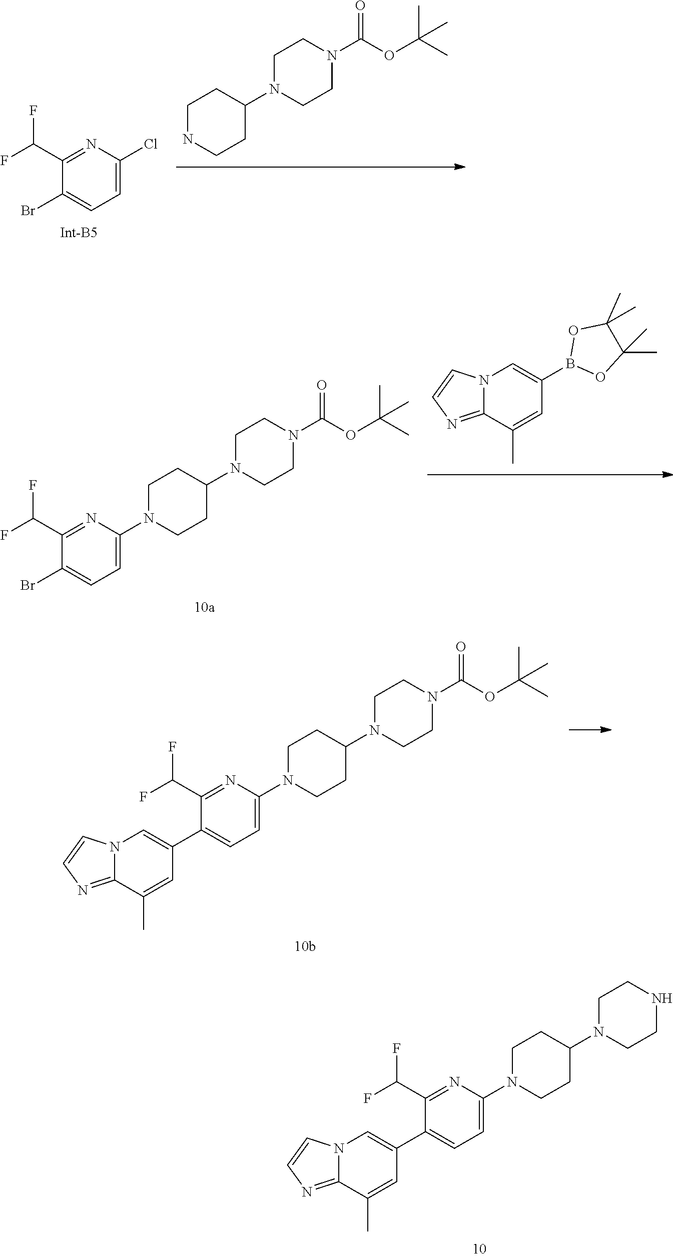

### Step 1: preparation of tert-butyl 4-[1-[5-bromo-6-(difluoromethyl)-2-pyridyl]-4-piperidyl]piperazine-1-carboxylate

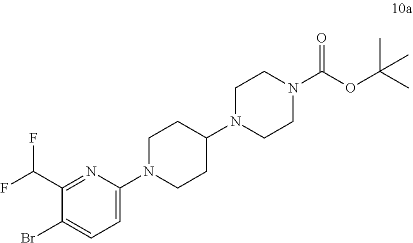 [SMILES: CC(C)(C)OC(=O)N1CCN(C2CCN(c3ccc(Br)c(C(F)F)n3)CC2)CC1]

A mixture of 3-bromo-6-chloro-2-(difluoromethyl)pyridine (70 mg, 289 mop, 4-(4-piperidyl)piperazine-1-carboxylic acid tert-butyl ester (100 mg, 371 μmol, CAS No. 205059-24-1, vendor: Bide Pharmatech, catalog BD57121) and K₂CO₃ (70 mg, 506 μmol) in DMF (2 mL) was stirred at 125° C. for 16 hours.

After the reaction was completed, the mixture was then concentrated in vacuo, the residue was then purified by flash column eluting with a gradient of MeOH/DCM (0% to 15%) to give tert-butyl 4-[1-[5-bromo-6-(difluoromethyl)-2-pyridyl]-4-piperidyl]piperazine-1-carboxylate (136 mg, 98% yield) as a yellow oil. MS: calc'd 476 (M+H⁺), measured 476 (M+H⁺).

### Step 2: preparation of tert-butyl 4-[1-[6-(difluoromethyl)-5-(8-methylimidazo[1,2-a]pyridin-6-yl)-2-pyridyl]-4-piperidyl]piperazine-1-carboxylate

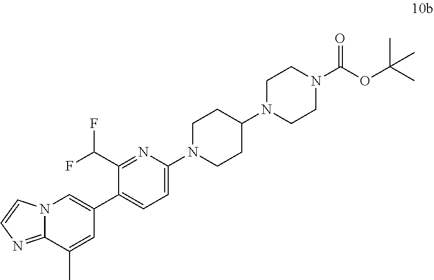 [SMILES: Cc1cc(-c2ccc(N3CCC(N4CCN(C(=O)OC(C)(C)C)CC4)CC3)nc2C(F)F)cn2ccnc12]

A mixture of 8-methyl-6-(4,4,5,5-tetramethyl-1,3,2-dioxaborolan-2-yl)imidazo[1,2-a]pyridine (80 mg, 310 μmol), tert-butyl 4-[1-[5-bromo-6-(difluoromethyl)-2-pyridyl]-4-piperidyl]piperazine-1-carboxylate (140 mg, 295 mol, CAS No. 205059-24-1, vendor: Bide Pharmatech, catalog BD57121) and K₂CO₃ (85.7 mg, 620 μmol) in a mixed solvent of dioxane (5 mL) and water (1 mL) was added PdCl₂(DPPF)-CH₂Cl₂ adduct (22.7 mg, 31 μmol) and the mixture was stirred at 80° C. under N₂ atmosphere for 16 hours.

After the reaction was completed, the mixture was then concentrated in vacuo. The residue was then purified by flash column eluting with a gradient of MeOH/DCM (0% to 10%) to give tert-butyl 4-[1-[6-(difluoromethyl)-5-(8-methylimidazo[1,2-a]pyridin-6-yl)-2-pyridyl]-4-piperidyl]piperazine-1-carboxylate (89 mg, 54.5% yield) as a yellow oil. MS: calc'd 527 (M+H⁺), measured 527 (M+H⁺).

### Step 3: preparation of 6-[2-(difluoromethyl)-6-(4-piperazin-1-yl-1-piperidyl)-3-pyridyl]-8-methyl-imidazo[1,2-a]pyridine

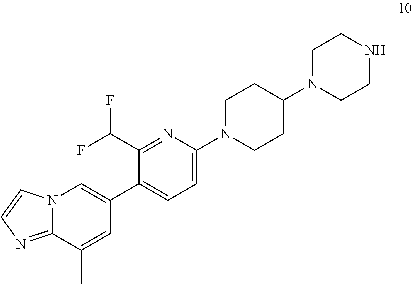 [SMILES: Cc1cc(-c2ccc(N3CCC(N4CCNCC4)CC3)nc2C(F)F)cn2ccnc12]

To a solution of tert-butyl 4-[1-[6-(difluoromethyl)-5-(8-methylimidazo[1,2-a]pyridin-6-yl)-2-pyridyl]-4-piperidyl]piperazine-1-carboxylate in DCM (4 mL) was added TFA (1 mL) and the mixture was then stirred at 25° C. for 1 hour.

After the reaction was completed, the mixture was concentrated in vacuo and the residue was then purified by Prep-HPLC to give 6-[2-(difluoromethyl)-6-(4-piperazin-1-yl-1-piperidyl)-3-pyridyl]-8-methyl-imidazo[1,2-a]pyridine (23 mg, 23.6% yield) as a light yellow powder. ¹H NMR (400 MHz, CD₃OD, 298 K) δ (ppm)=8.23-8.26 (m, 1H), 7.85 (d, J=1.3 Hz, 1H), 7.55-7.61 (m, 2H), 7.07 (s, 1H), 6.99 (d, J=8.8 Hz, 1H), 6.38-6.68 (m, 1H), 4.55 (br d, J=13.3 Hz, 2H), 2.83-2.94 (m, 6H), 2.49-2.67 (m, 8H), 2.00 (br d, J=12.5 Hz, 2H), 1.45-1.58 (m, 2H). MS: calc'd 427 (M+H⁺), measured 427 (M+H⁺).

### Example 11

### 6-[2-(difluoromethyl)-6-(4-piperazin-1-yl-1-piperidyl)-3-pyridyl]-2,8-dimethyl-imidazo[1,2-a]pyridine

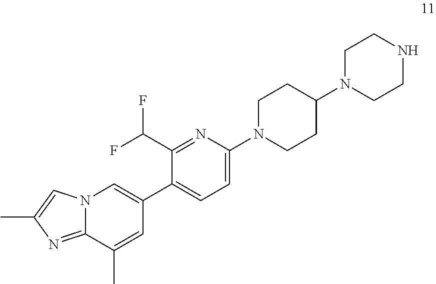 [SMILES: Cc1cn2cc(-c3ccc(N4CCC(N5CCNCC5)CC4)nc3C(F)F)cc(C)c2n1]

### Step 1: preparation of 2,8-dimethyl-6-(4,4,5,5-tetramethyl-1,3,2-dioxaborolan-2-yl)imidazo[1,2-a]pyridine

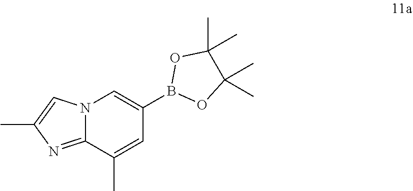

A mixture of 6-bromo-2,8-dimethylimidazo[1,2-a]pyridine (2.25 g, 10 mmol), [1,1′-bis(diphenylphosphino)ferrocene]dichloropalladium(II) (817 mg, 1 mmol), bis(pinacolato)diboron (3.05 g, 12 mmol, CAS No. 73183-34-3, vendor: Accela ChemBio Inc, catalog: SY001323) and potassium acetate (2.45 g, 25 mmol) in 1,4-dioxane (8.5 mL) was stirred at 90° C. under argon atmosphere for 2 hours. After the reaction was completed, the mixture was concentrated in vacuo to afford the crude product of 2,8-dimethyl-6-(4,4,5,5-tetramethyl-1,3,2-dioxaborolan-2-yl)imidazo[1,2-a]pyridine as a brown oil, which was used in the next step without further purification. MS: calc'd 273 (M+H⁺), measured 273 (M+H⁺).

### Step 2: preparation of 6-[2-(difluoromethyl)-6-(4-piperazin-1-yl-1-piperidyl)-3-pyridyl]-2,8-dimethyl-imidazo[1,2-a]pyridine

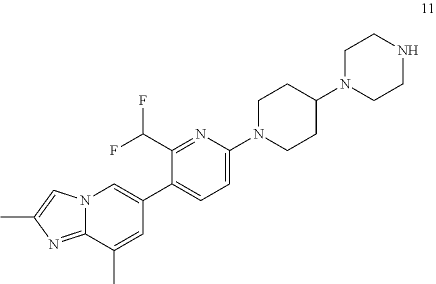 [SMILES: Cc1cn2cc(-c3ccc(N4CCC(N5CCNCC5)CC4)nc3C(F)F)cc(C)c2n1]

The title compound was prepared in analogy to the preparation of Example 10 by using 2,8-dimethyl-6-(4,4,5,5-tetramethyl-1,3,2-dioxaborolan-2-yl)imidazo[1,2-a]pyridine instead of 8-methyl-6-(4,4,5,5-tetramethyl-1,3,2-dioxaborolan-2-yl)imidazo[1,2-a]pyridine in Step 2.

Example 11 (24 mg) was obtained as a yellow solid. ¹H NMR (400 MHz, CD₃OD, 298 K) δ (ppm)=8.53 (s, 1H), 7.97 (d, J=1.1 Hz, 1H), 7.67-7.70 (m, 1H), 7.62-7.67 (m, 1H), 7.09 (d, J=8.8 Hz, 1H), 6.39-6.72 (m, 1H), 4.68 (br d, J=13.6 Hz, 2H), 3.48-3.55 (m, 4H), 3.35-3.47 (m, 5H), 3.01 (br t, J=12.0 Hz, 2H), 2.66 (s, 3H), 2.57-2.61 (m, 3H), 2.18 (br d, J=11.1 Hz, 2H), 1.72 (qd, J=12.1, 4.0 Hz, 2H). MS: calc'd 441 (M+H⁺), measured 441 (M+H⁺).

### Example 12

### 1-[1-[6-(difluoro methyl)-5-(2,8-dimethylimidazo[1,2-a]pyridin-6-yl)-2-pyridyl]-4-piperidyl]-3-methyl-azetidin-3-amine

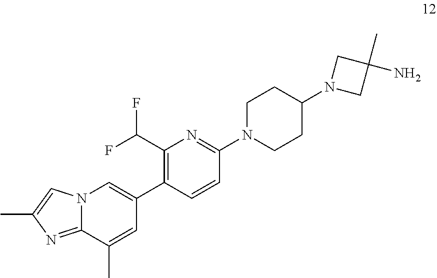 [SMILES: Cc1cn2cc(-c3ccc(N4CCC(N5CC(C)(N)C5)CC4)nc3C(F)F)cc(C)c2n1]

The title compound was prepared according to the following scheme:

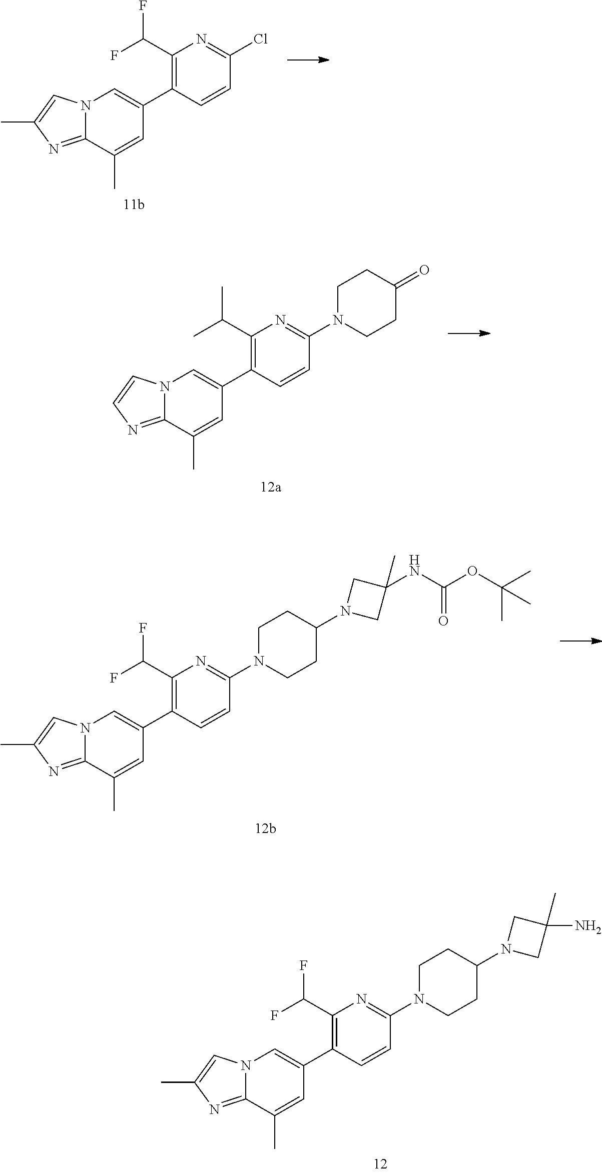

### Step 1: preparation of 1-[6-(difluoromethyl)-5-(2,8-dimethylimidazo[1,2-a]pyridin-6-yl)-2-pyridyl]piperidin-4-one

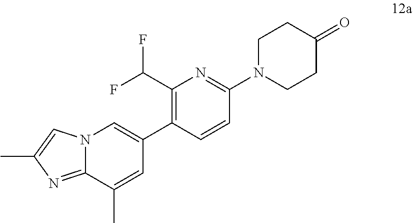 [SMILES: Cc1cn2cc(-c3ccc(N4CCC(=O)CC4)nc3C(F)F)cc(C)c2n1]

To a mixture of 6-[6-chloro-2-(difluoromethyl)-3-pyridyl]-2,8-dimethyl-imidazo[1,2-a]pyridine (462 mg, 1.50 mmol), piperidin-4-one hydrochloride (305 mg, 2.25 mmol), and cesium carbonate (2.20 g, 6.75 mmol) in 1,4-dioxane (10 mL) was added RuPhos Pd G2 (175 mg, 225 μmol), and the mixture was stirred at 110° C. under argon atmosphere for 16 hours.

The reaction mixture was filtered through a pad of Celite, and the filtrate was concentrated in vacuo to give the crude material as a brown oil. The residue was purified by flash chromatography (silica gel) eluting with 0-30% MeOH in DCM to give 1-[6-(difluoromethyl)-5-(2, 8-dimethylimidazo[1,2-a]pyridin-6-yl)-2-pyridyl]piperidin-4-one (131 mg, 23.58% yield) as a light brown solid. MS: cal'd 371 (M+H⁺), measured 371 (M+H⁺).

### Step 2: preparation of tert-butyl N-[1-[1-[6-(difluoromethyl)-5-(2,8-dimethylimidazo[1,2-a]pyridin-6-yl)-2-pyridyl]-4-piperidyl]-3-methyl-azetidin-3-yl]carbamate

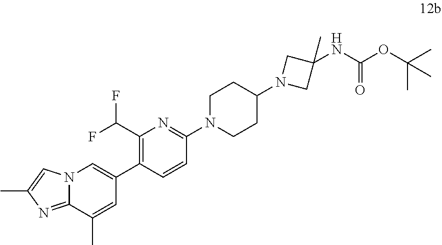 [SMILES: Cc1cn2cc(-c3ccc(N4CCC(N5CC(C)(NC(=O)OC(C)(C)C)C5)CC4)nc3C(F)F)cc(C)c2n1]

To a solution of tert-butyl N-(3-methylazetidin-3-yl)carbamate hydrochloride (38.8 mg, 174 μmol, CAS No. 1408076-37-8, vendor: Pharmablock, catalog: PB03046-01), 1-[6-(difluoromethyl)-5-(2, 8-dimethylimidazo[1,2-a]pyridin-6-yl)-2-pyridyl]piperidin-4-one (43 mg, 116 μmol) in EtOH (1.0 mL) was added acetic acid (10.5 mg, 10.0 μL, 174 μmol), and the reaction mixture was stirred at 50° C. for 1 hour. Then to the resulting mixture was added sodium cyanoborohydride (21.9 mg, 348 μmol), and the mixture was further stirred at 50° C. under argon atmosphere for 16 hours.

The reaction mixture was quickly passed through a flash column eluting with 0-40% MeOH in DCM to afford the crude tert-butyl N-[1-[1-[6-(difluoromethyl)-5-(2,8-dimethylimidazo[1,2-a]pyridin-6-yl)-2-pyridyl]-4-piperidyl]-3-methyl-azetidin-3-yl]carbamate as a yellow oil, which was used in the next step without further purification. MS: calc'd 541 (M+H⁺), measured 541 (M+H⁺).

### Step 3: preparation of 1-[1-[6-(difluoromethyl)-5-(2,8-dimethylimidazo[1,2-a]pyridin-6-yl)-2-pyridyl]-4-piperidyl]-3-methyl-azetidin-3-amine

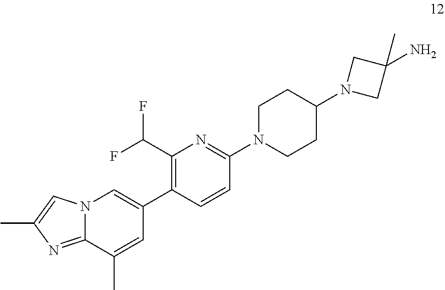 [SMILES: Cc1cn2cc(-c3ccc(N4CCC(N5CC(C)(N)C5)CC4)nc3C(F)F)cc(C)c2n1]

To a solution of tert-butyl N-[1-[1-[6-(difluoromethyl)-5-(2,8-dimethylimidazo[1,2-a]pyridin-6-yl)-2-pyridyl]-4-piperidyl]-3-methyl-azetidin-3-yl]carbamate from the previous step in DCM (2.0 mL) was added TFA (1.14 g, 0.77 mL, 10 mmol), and the reaction mixture was stirred at room temperature under argon atmosphere for 16 hours.

The mixture was concentrated in vacuo and the residue was purified by Prep-HPLC to give 1-[1-[6-(difluoromethyl)-5-(2,8-dimethylimidazo[1,2-a]pyridin-6-yl)-2-pyridyl]-4-piperidyl]-3-methyl-azetidin-3-amine (10.5 mg, 23.83% yield over 2 steps) as a white powder. ¹H NMR (400 MHz, CD₃OD) δ 8.17-8.09 (m, 1H), 7.59 (s, 1H), 7.58-7.52 (m, 1H), 7.02 (s, 1H), 7.00-6.94 (m, 1H), 6.52 (t, J_{H-F}=54.5 Hz, 1H), 4.51-4.34 (m, 2H), 3.38-3.32 (m, 2H), 3.07 (d, J=8.2 Hz, 2H), 3.00-2.89 (m, 2H), 2.55 (s, 3H), 2.50-2.37 (m, 4H), 1.85 (br d, J=10.5 Hz, 2H), 1.42 (s, 3H), 1.34-1.20 (m, 2H). MS: calc'd 441 (M+H⁺), measured 441 (M+H⁺).

### Example 13

### 3-[6-(difluoromethyl)-5-(8-methoxyimidazo[1,2-a]pyridin-6-yl)-2-pyridyl]-3,9-diazaspiro[5.5]undecane

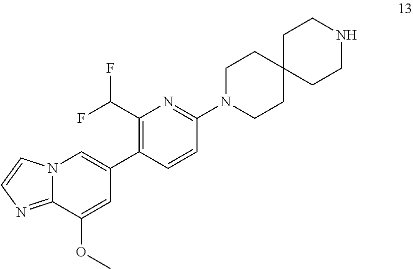 [SMILES: COc1cc(-c2ccc(N3CCC4(CCNCC4)CC3)nc2C(F)F)cn2ccnc12]

### Step 1: preparation of 8-methoxy-6-(4,4,5,5-tetramethyl-1,3,2-dioxaborolan-2-yl)imidazo[1,2-a]pyridine

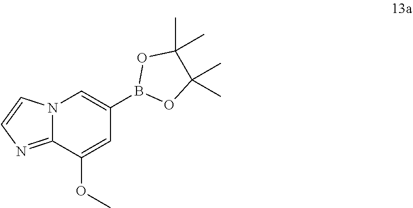 [SMILES: COc1cc(B2OC(C)(C)C(C)(C)O2)cn2ccnc12]

Compound 13a was prepared in analogy to the preparation of compound 9a by using 6-bromo-8-methoxy-imidazo[1,2-a]pyridine instead of 6-bromo-8-methylimidazo[1,2-a]pyridine. 8-methoxy-6-(4,4,5,5-tetramethyl-1,3,2-dioxaborolan-2-yl)imidazo[1,2-a]pyridine (380 mg) was obtained as a brown oil. MS calc'd 275 (M+H⁺), measured 275 (M+H⁺).

### Step 2: preparation of tert-butyl 9-[5-bromo-6-(difluoromethyl)-2-pyridyl]-3,9-diazaspiro[5.5]undecane-3-carboxylate

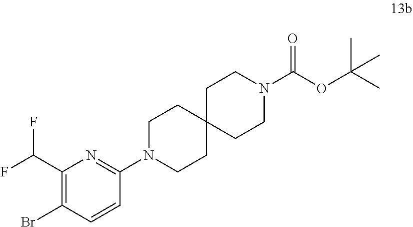 [SMILES: CC(C)(C)OC(=O)N1CCC2(CC1)CCN(c1ccc(Br)c(C(F)F)n1)CC2]

Compound 13b was prepared in analogy to the preparation of compound 10b by using tert-butyl 3,9-diazaspiro[5.5]undecane-3-carboxylate instead of 4-(4-piperidyl)piperazine-1-carboxylic acid tert-butyl ester in Step 1. MS: calc'd 460 (M+H⁺), measured 459 (M+H⁺).

### Step 3: preparation of 3-[6-(difluoromethyl)-5-(8-methoxyimidazo[1,2-a]pyridin-6-yl)-2-pyridyl]-3,9-diazaspiro[5.5]undecane

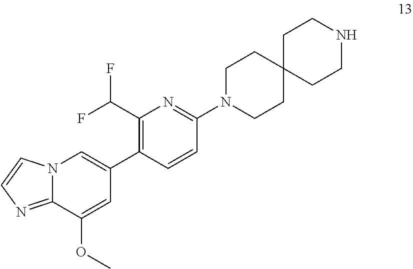 [SMILES: COc1cc(-c2ccc(N3CCC4(CCNCC4)CC3)nc2C(F)F)cn2ccnc12]

The title compound was prepared in analogy to the preparation of Example 10 By using 8-methoxy-6-(4,4,5,5-tetramethyl-1,3,2-dioxaborolan-2-yl)imidazo[1,2-a]pyridine and tert-butyl 9-[5-bromo-6-(difluoromethyl)-2-pyridyl]-3,9-diazaspiro[5.5]undecane-3-carboxylate instead of 8-methyl-6-(4,4,5,5-tetramethyl-1,3,2-dioxaborolan-2-yl)imidazo[1,2-a]pyridine and tert-butyl 4-[1-[5-(1,5-dimethyl-6-oxo-3-pyridyl)-6-isopropyl-2-pyridyl]-4-piperidyl]piperazine-1-carboxylate in Step 2.

Example 13 (12 mg, 17.9% yield) was obtained as a yellow solid. ¹H NMR (400 MHz, CD₃OD, 298 K) δ (ppm)=8.35 (d, J=1.1 Hz, 1H), 8.23 (d, J=2.1 Hz, 1H), 8.03 (d, J=2.1 Hz, 1H), 7.67 (d, J=8.8 Hz, 1H), 7.33-7.36 (m, 1H), 7.03 (d, J=8.8 Hz, 1H), 6.42-6.75 (m, 1H), 4.13-4.19 (m, 3H), 3.70-3.78 (m, 4H), 3.17-3.27 (m, 4H), 1.78-1.85 (m, 4H), 1.65-1.73 (m, 4H). MS: calc'd 428 (M+H⁺), measured 428 (M+H⁺).

### Example 14

### 2-[1-[6-ethyl-5-(8-methylimidazo[1,2-a]pyridin-6-yl)-2-pyridyl]-4-piperidyl]-5-oxa-2,8-diazaspiro[3.5]nonane

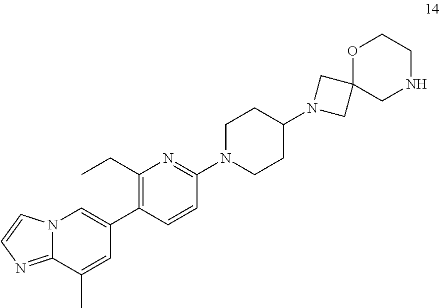 [SMILES: CCc1nc(N2CCC(N3CC4(CNCCO4)C3)CC2)ccc1-c1cc(C)c2nccn2c1]

### Step 1: preparation of 6-(6-chloro-2-ethyl-3-pyridyl)-8-methyl-imidazo[1,2-a]pyridine

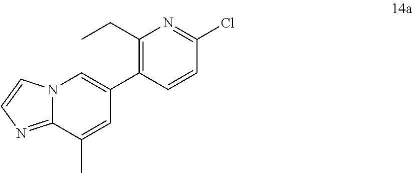 [SMILES: CCc1nc(Cl)ccc1-c1cc(C)c2nccn2c1]

Compound 14a was prepared in analogy to the preparation of compound 1a by using 6-chloro-2-ethyl-3-(4,4,5,5-tetramethyl-1,3,2-dioxaborolan-2-yl)pyridine (Int-B4) instead of 6-chloro-2-isopropyl-3-(4,4,5,5-tetramethyl-1,3,2-dioxaborolan-2-yl)pyridine (Int-B2) in step 1. Step 2: preparation of 1-[6-ethyl-5-(8-methylimidazo[1,2-a]pyridin-6-yl)-2-pyridyl]piperidin-4-one

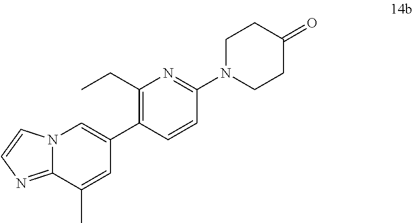 [SMILES: CCc1nc(N2CCC(=O)CC2)ccc1-c1cc(C)c2nccn2c1]

To a mixture of 6-(6-chloro-2-ethylpyridin-3-yl)-8-methylimidazo[1,2-a]pyridine (544 mg, 2.00 mmol), piperidin-4-one hydrochloride (407 mg, 3.00 mmol) and cesium carbonate (2.93 g, 9.00 mmol) in 1,4-Dioxane (20 mL) was added RuPhos Pd G2 (233 mg, 300 μmol) and the mixture was then stirred at 110° C. under Argon atmosphere for 16 hours.

The reaction mixture was filtered through a pad of Celite, and the filtrate was concentrated in vacuo to give the crude product as a brown oil. The residue was purified by flash chromatography eluting with 0-30% MeOH in DCM to give 1-[6-ethyl-5-(8-methylimidazo[1,2-a]pyridin-6-yl)-2-pyridyl]piperidin-4-one (106 mg, 15.85% yield) as a dark brown solid. MS: calc'd 335 (M+H⁺), measured 335 (M+H⁺).

### Step 3: preparation of tert-butyl 2-[1-[6-ethyl-5-(8-methylimidazo[1,2-a]pyridin-6-yl)-2-pyridyl]-4-piperidyl]-5-oxa-2,8-diazaspiro[3.5]nonane-8-carboxylate

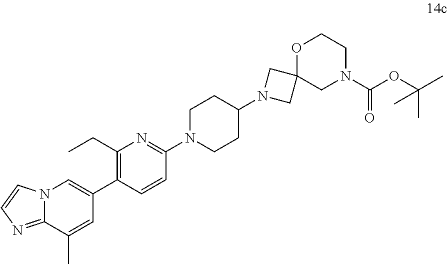 [SMILES: CCc1nc(N2CCC(N3CC4(CN(C(=O)OC(C)(C)C)CCO4)C3)CC2)ccc1-c1cc(C)c2nccn2c1]

To a solution of tert-butyl 5-oxa-2,8-diazaspiro[3.5]nonane-8-carboxylate (54.2 mg, 0.238 mmol, CAS No. 1251005-61-4, vendor: Pharmablock, catalog: PBN20111065), 1-[6-ethyl-5-(8-methylimidazo[1,2-a]pyridin-6-yl)-2-pyridyl]-4-piperidone (53.0 mg, 0.158 mmol) in 1.6 mL EtOH was added acetic acid (19.0 mg, 18.1 μL, 0.317 mmol) and the reaction mixture was stirred at 50° C. for 1 hour. Then to the resulting mixture was added sodium cyanoborohydride (19.9 mg, 0.317 mmol) and the mixture was further stirred at 50° C. under argon atmosphere for 16 hours.

The reaction mixture was quickly passed through a flash column eluting with 0-40% MeOH in DCM to afford the crude of tert-butyl 2-[1-[6-ethyl-5-(8-methylimidazo[1,2-a]pyridin-6-yl)-2-pyridyl]-4-piperidyl]-5-oxa-2,8-diazaspiro[3.5]nonane-8-carboxylate as a light yellow oil, which was used in the next step without further purification. MS: calc'd 547 (M+H⁺), measured 547 (M+H⁺).

### Step 4: 2-[1-[6-ethyl-5-(8-methylimidazo[1,2-a]pyridin-6-yl)-2-pyridyl]-4-piperidyl]-5-oxa-2,8-diazaspiro[3.5]nonane

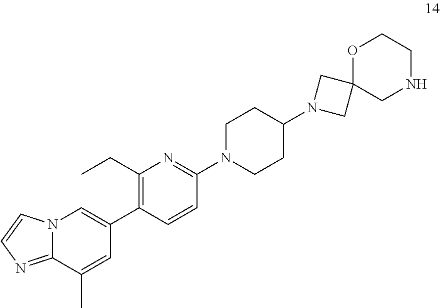 [SMILES: CCc1nc(N2CCC(N3CC4(CNCCO4)C3)CC2)ccc1-c1cc(C)c2nccn2c1]

To a solution of tert-butyl 2-[1-[6-ethyl-5-(8-methylimidazo[1,2-a]pyridin-6-yl)-2-pyridyl]-4-piperidyl]-5-oxa-2,8-diazaspiro[3.5]nonane-8-carboxylate from the previous step in DCM (3.1 mL) was added TFA (1.80 g, 1.21 mL) and the reaction mixture was stirred at room temperature under argon atmosphere for 16 hours.

The mixture was concentrated in vacuo and the residue was purified by Prep-HPLC to give 2-[1-[6-ethyl-5-(8-methylimidazo[1,2-a]pyridin-6-yl)-2-pyridyl]-4-piperidyl]-5-oxa-2,8-diazaspiro[3.5]nonane (13.5 mg, 15.24% yield over 2 steps) as a light yellow solid. ¹H NMR (400 MHz, CD₃OD) δ 8.62 (s, 1H), 8.23 (d, J=2.1 Hz, 1H), 8.09 (d, J=2.1 Hz, 1H), 7.73 (s, 1H), 7.57 (d, J=8.7 Hz, 1H), 6.90 (d, J=8.8 Hz, 1H), 4.62 (br d, J=13.8 Hz, 2H), 4.44 (br d, J=11.6 Hz, 2H), 4.35 (d, J=12.0 Hz, 2H), 4.07-4.00 (m, 2H), 3.61-3.57 (m, 1H), 3.56 (s, 2H), 3.29-3.27 (m, 2H), 3.02 (br t, J=12.2 Hz, 2H), 2.74-2.66 (m, 5H), 2.14 (br d, J=12.1 Hz, 2H), 1.55 (qd, J=12.1, 4.2 Hz, 2H), 1.21 (t, J=7.5 Hz, 3H). MS: calc'd 447 (M+H⁺), measured 447 (M+H⁺).

### Example 15

### (3R,4R)-1-[1-[6-ethyl-5-(8-methylimidazo[1,2-a]pyridin-6-yl)-2-pyridyl]-4-piperidyl]-4-methoxy-pyrrolidin-3-amine

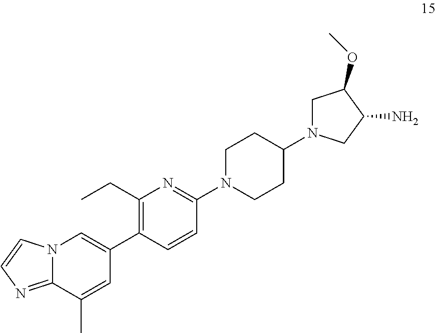 [SMILES: CCc1nc(N2CCC(N3C[C@@H](N)[C@H](OC)C3)CC2)ccc1-c1cc(C)c2nccn2c1]

The title compound was prepared in analogy to the preparation of Example 14 by using tert-butyl N-[(3R,4R)-4-methoxypyrrolidin-3-yl]carbamate (CAS No. 1932066-52-8, vendor: Pharmablock, catalog: PBZ4728) instead of tert-butyl 5-oxa-2,8-diazaspiro[3.5]nonane-8-carboxylate in Step 3.

Example 15 (8.7 mg) was obtained as a light yellow solid. ¹H NMR (400 MHz, CD₃OD) δ 8.63-8.61 (m, 1H), 8.23 (d, J=2.2 Hz, 1H), 8.09 (d, J=2.1 Hz, 1H), 7.75-7.72 (m, 1H), 7.58 (d, J=8.8 Hz, 1H), 6.92 (d, J=8.8 Hz, 1H), 4.62 (br d, J=13.3 Hz, 2H), 4.26-4.22 (m, 1H), 4.12-3.97 (m, 2H), 3.82-3.67 (m, 2H), 3.55 (br dd, J=10.9, 6.3 Hz, 2H), 3.46 (s, 3H), 3.03 (br t, J=12.0 Hz, 2H), 2.75-2.67 (m, 5H), 2.25 (br s, 2H), 1.86-1.73 (m, 2H), 1.21 (t, J=7.5 Hz, 3H). MS: calc'd 435 (M+H⁺), measured 435 (M+H⁺).

### Example 16

### 2-[1-[5-(2,8-dimethylimidazo[1,2-a]pyridin-6-yl)-6-ethyl-2-pyridyl]-4-piperidyl]-5-oxa-2,8-diazaspiro[3.5]nonane

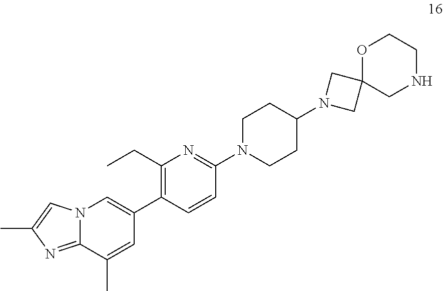 [SMILES: CCc1nc(N2CCC(N3CC4(CNCCO4)C3)CC2)ccc1-c1cc(C)c2nc(C)cn2c1]

### Step 1: preparation of 6-(6-chloro-2-ethyl-3-pyridyl)-2,8-dimethyl-imidazo[1,2-a]pyridine

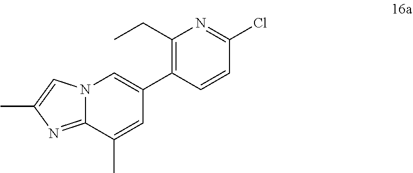 [SMILES: CCc1nc(Cl)ccc1-c1cc(C)c2nc(C)cn2c1]

Compound 16a was prepared in analogy to the preparation of compound 1a by using 6-chloro-2-ethyl-3-(4,4,5,5-tetramethyl-1,3,2-dioxaborolan-2-yl)pyridine (Int-B4) and 6-bromo-2,8-dimethyl-imidazo[1,2-a]pyridine (Int-A1) instead of 6-chloro-2-isopropyl-3-(4,4,5,5-tetramethyl-1,3,2-dioxaborolan-2-yl)pyridine (Int-B2) and 6-bromo-8-methyl-imidazo[1,2-a]pyridine in step 1.

### Step 2: preparation of 2-[1-[5-(2,8-dimethylimidazo[1,2-a]pyridin-6-yl)-6-ethyl-2-pyridyl]-4-piperidyl]-5-oxa-2,8-diazaspiro[3.5]nonane

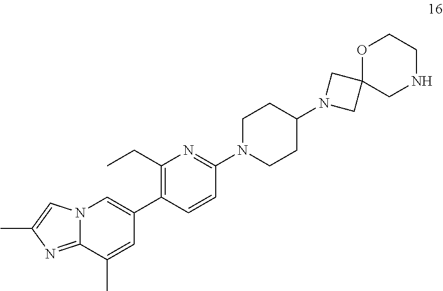 [SMILES: CCc1nc(N2CCC(N3CC4(CNCCO4)C3)CC2)ccc1-c1cc(C)c2nc(C)cn2c1]

The title compound was prepared in analogy to the preparation of Example 14 by using 6-(6-chloro-2-ethyl-3-pyridyl)-2,8-dimethyl-imidazo[1,2-a]pyridine instead of 6-(6-chloro-2-ethylpyridin-3-yl)-8-methylimidazo[1,2-a]pyridine in Step 2.

Example 16 (25.7 mg) was obtained as a light yellow solid. ¹H NMR (400 MHz, CD₃OD) δ 8.51 (s, 1H), 7.94 (d, J=1.0 Hz, 1H), 7.66 (s, 1H), 7.55 (d, J=8.7 Hz, 1H), 6.90 (d, J=8.8 Hz, 1H), 4.61 (br d, J=13.6 Hz, 2H), 4.44 (br d, J=11.6 Hz, 2H), 4.40-4.30 (m, 2H), 4.08-3.98 (m, 2H), 3.61-3.51 (m, 3H), 3.30-3.22 (m, 2H), 3.02 (br t, J=12.3 Hz, 2H), 2.73-2.64 (m, 5H), 2.61-2.56 (m, 3H), 2.14 (br d, J=12.2 Hz, 2H), 1.55 (br dd, J=12.1, 3.8 Hz, 2H), 1.20 (t, J=7.5 Hz, 3H). MS: calc'd 461 (M+H⁺), measured 461 (M+H⁺).

### Example 17

### (3R,4R)-1-[1-[5-(2,8-dimethylimidazo[1,2-a]pyridin-6-yl)-6-ethyl-2-pyridyl]-4-piperidyl]-4-methoxy-pyrrolidin-3-amine

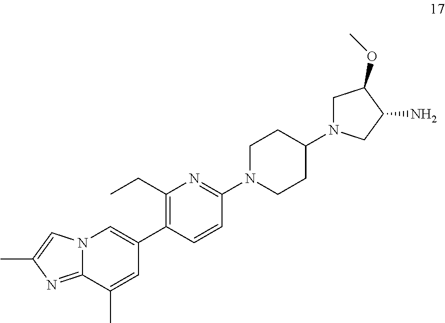 [SMILES: CCc1nc(N2CCC(N3C[C@@H](N)[C@H](OC)C3)CC2)ccc1-c1cc(C)c2nc(C)cn2c1]

The title compound was prepared in analogy to the preparation of Example 14 by using 6-(6-chloro-2-ethyl-3-pyridyl)-2,8-dimethyl-imidazo[1,2-a]pyridine instead of 6-(6-chloro-2-ethylpyridin-3-yl)-8-methylimidazo[1,2-a]pyridine in Step 2 and using tert-butyl N-[(3R,4R)-4-methoxypyrrolidin-3-yl]carbamate (CAS No. 1932066-52-8, vendor: Pharmablock, catalog: PBZ4728) instead of tert-butyl 5-oxa-2,8-diazaspiro[3.5]nonane-8-carboxylate in Step 3.

Example 17 (44.5 mg) was obtained as a light yellow solid. ¹H NMR (400 MHz, CD₃OD) δ 8.60 (s, 1H), 7.98 (d, J=1.0 Hz, 1H), 7.82-7.74 (m, 1H), 7.71-7.66 (m, 1H), 7.18 (d, J=9.2 Hz, 1H), 4.55 (br d, J=13.6 Hz, 2H), 4.32-4.24 (m, 1H), 4.20-4.09 (m, 1H), 4.09-3.99 (m, 1H), 3.90-3.73 (m, 2H), 3.72-3.59 (m, 2H), 3.46 (s, 3H), 3.29-3.18 (m, 2H), 2.81 (q, J=7.5 Hz, 2H), 2.68 (s, 3H), 2.61-2.57 (m, 3H), 2.43-2.23 (m, 2H), 2.00-1.82 (m, 2H), 1.21 (t, J=7.5 Hz, 3H). MS: calc'd 449 (M+H⁺), measured 449 (M+H⁺).

### Example 18

### [4-[5-(2,8-dimethylimidazo[1,2-a]pyridin-6-yl)-6-ethyl-2-pyridyl]piperazin-1-yl]-morpholin-2-yl-methanone

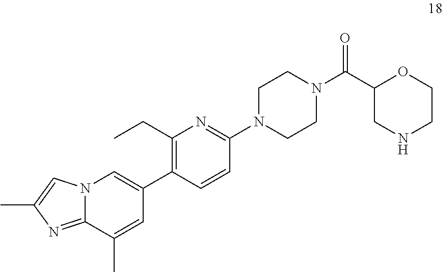 [SMILES: CCc1nc(N2CCN(C(=O)C3CNCCO3)CC2)ccc1-c1cc(C)c2nc(C)cn2c1]

The title compound was prepared according to the following scheme:

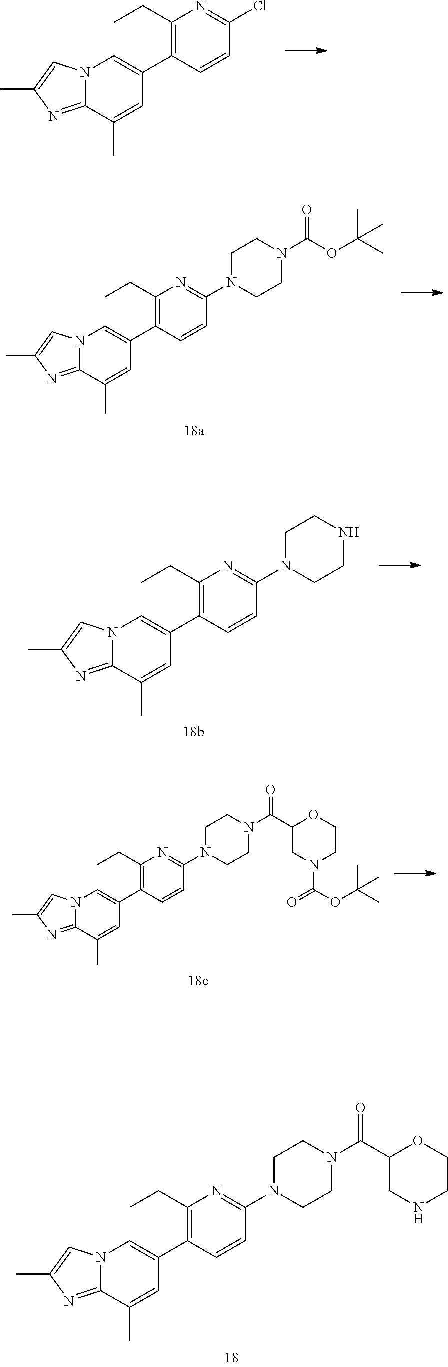

### Step 1: preparation of tert-butyl 4-[5-(2,8-dimethylimidazo[1,2-a]pyridin-6-yl)-6-ethyl-2-pyridyl]piperazine-1-carboxylate

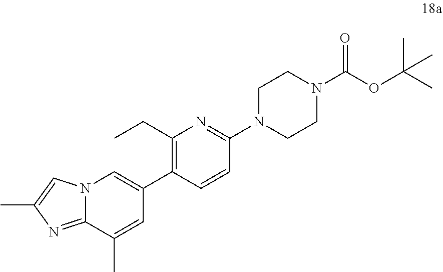 [SMILES: CCc1nc(N2CCN(C(=O)OC(C)(C)C)CC2)ccc1-c1cc(C)c2nc(C)cn2c1]

To a mixture of 6-(6-chloro-2-ethylpyridin-3-yl)-2,8-dimethylimidazo[1,2-a]pyridine (357 mg, 1.25 mmol), 1-boc-piperazine (349 mg, 1.88 mmol) and cesium carbonate (1.22 g, 3.75 mmol) in 1,4-dioxane (8 mL) was added RuPhos Pd G2 (146 mg, 0.188 mmol) and the mixture was then stirred at 110° C. under argon atmosphere for 16 hours.

After cooling to room temperature, the reaction mixture was filtered through a pad of Celite, and concentrated in vacuo. The residue was purified by flash chromatography eluting with 0-40% EtOAc (containing 10% MeOH) in petroleum ether to give tert-butyl 4-[5-(2,8-dimethylimidazo[1,2-a]pyridin-6-yl)-6-ethyl-2-pyridyl]piperazine-1-carboxylate (321 mg, 58.96% yield) as a light yellow solid. MS: calc'd 436 (M+H⁺), measured 436 (M+H⁺).

### Step 2: preparation of 6-(2-ethyl-6-piperazin-1-yl-3-pyridyl)-2,8-dimethyl-imidazo[1,2-a]pyridine

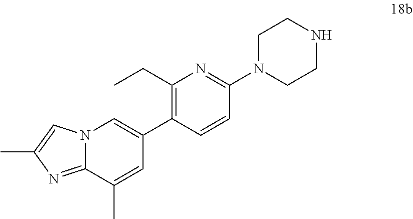 [SMILES: CCc1nc(N2CCNCC2)ccc1-c1cc(C)c2nc(C)cn2c1]

To a solution of tert-butyl 4-[5-(2,8-dimethylimidazo[1,2-a]pyridin-6-yl)-6-ethyl-2-pyridyl]piperazine-1-carboxylate (321 mg, 0.737 mmol) in DCM (15 mL) was added TFA (4.20 g, 2.82 mL, 36.9 mmol) and the reaction mixture was stirred at room temperature under argon atmosphere for 16 hours.

The reaction mixture was concentrated in vacuo and then partitioned between DCM (100 mL) and saturated K₂CO₃ (100 mL). The organic layer was separated out and washed with saturated K₂CO₃ (50 mL) three times, dried over Na₂SO₄ and concentrated in vacuo. The residue was purified by flash chromatography eluting with 0-80% MeOH in DCM to give 6-(2-ethyl-6-piperazin-1-yl-3-pyridyl)-2,8-dimethyl-imidazo[1,2-a]pyridine (118 mg, 47.73% yield) as an off-white solid. MS: calc'd 336 (M+H⁺), measured 336 (M+H⁺).

### Step 3: preparation of tert-butyl 2-[4-[5-(2,8-dimethylimidazo[1,2-a]pyridin-6-yl)-6-ethyl-2-pyridyl]piperazine-1-carbonyl]morpholine-4-carboxylate

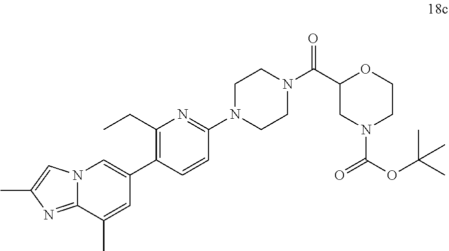 [SMILES: CCc1nc(N2CCN(C(=O)C3CN(C(=O)OC(C)(C)C)CCO3)CC2)ccc1-c1cc(C)c2nc(C)cn2c1]

To a solution of 6-(2-ethyl-6-piperazin-1-yl-3-pyridyl)-2,8-dimethyl-imidazo[1,2-a]pyridine (39.0 mg, 0.116 mmol), 4-boc-2-morpholinecarboxylic acid (40.3 mg, 0.174 mmol, CAS no. 189321-66-2, vendor: Pharmablock, catalog: PBN20121307), DIPEA (60.1 mg, 81 μL, 0.465 mmol) in DCM (3 mL) was added EDCI (33.4 mg, 0.174 mmol). The reaction mixture was stirred at room temperature under argon atmosphere for 16 hours. After the reaction was completed, the mixture was quickly passed through a flash column eluting with 0-40% MeOH in DCM to afford the crude tert-butyl 2-[4-[5-(2,8-dimethylimidazo[1,2-a]pyridin-6-yl)-6-ethyl-2-pyridyl]piperazine-1-carbonyl]morpholine-4-carboxylate as a yellow oil, which was used in the next step without further purification. MS: calc'd 549 (M+H⁺), measured 549 (M+H⁺).

### Step 4: preparation of [4-[5-(2,8-dimethylimidazo[1,2-a]pyridin-6-yl)-6-ethyl-2-pyridyl]piperazin-1-yl]-morpholin-2-yl-methanone

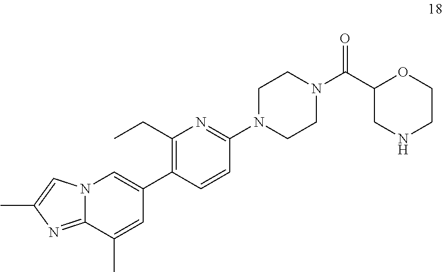 [SMILES: CCc1nc(N2CCN(C(=O)C3CNCCO3)CC2)ccc1-c1cc(C)c2nc(C)cn2c1]

To a solution of tert-butyl 2-[4-[5-(2,8-dimethylimidazo[1,2-a]pyridin-6-yl)-6-ethyl-2-pyridyl]piperazine-1-carbonyl]morpholine-4-carboxylate (prepared above) in DCM (2.3 mL) was added FA (1.33 g, 0.900 mL, 11.6 mmol). The reaction mixture was stirred at room temperature under argon atmosphere for 16 hours. The mixture was concentrated in vacuo and the residue was purified by Prep-HPLC to give [4-[5-(2,8-dimethylimidazo[1,2-a]pyridin-6-yl)-6-ethyl-2-pyridyl]piperazin-1-yl]-morpholin-2-yl-methanone (25.7 mg, 49.39% yield over 2 steps) as a white powder. ¹H NMR (400 MHz, CD₃OD) δ 8.10-8.08 (m, 1H), 7.58-7.56 (m, 1H), 7.43 (d, J=8.6 Hz, 1H), 6.99 (s, 1H), 6.71 (d, J=8.7 Hz, 1H), 4.37 (dd, J=8.1, 3.8 Hz, 1H), 3.94-3.85 (m, 1H), 3.85-3.45 (m, 9H), 3.04-2.92 (m, 2H), 2.85 (br d, J=3.3 Hz, 2H), 2.67 (q, J=7.5 Hz, 2H), 2.55 (s, 3H), 2.45-2.42 (m, 3H), 1.19 (t, J=7.5 Hz, 3H). MS: calc'd 449 (M+H⁺), measured 449 (M+H⁺).

### Example 19

### 2-[4-[5-(2,8-dimethylimidazo[1,2-a]pyridin-6-yl)-6-ethyl-2-pyridyl]piperazin-1-yl]-5,6,7,8-tetrahydropyrido[3,4-d]pyrimidine

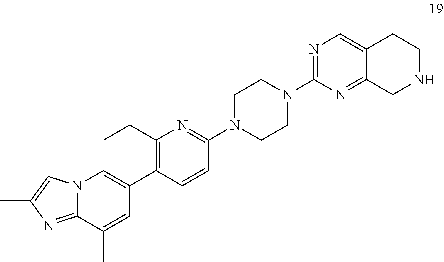 [SMILES: CCc1nc(N2CCN(c3ncc4c(n3)CNCC4)CC2)ccc1-c1cc(C)c2nc(C)cn2c1]

The title compound was prepared according to the following scheme:

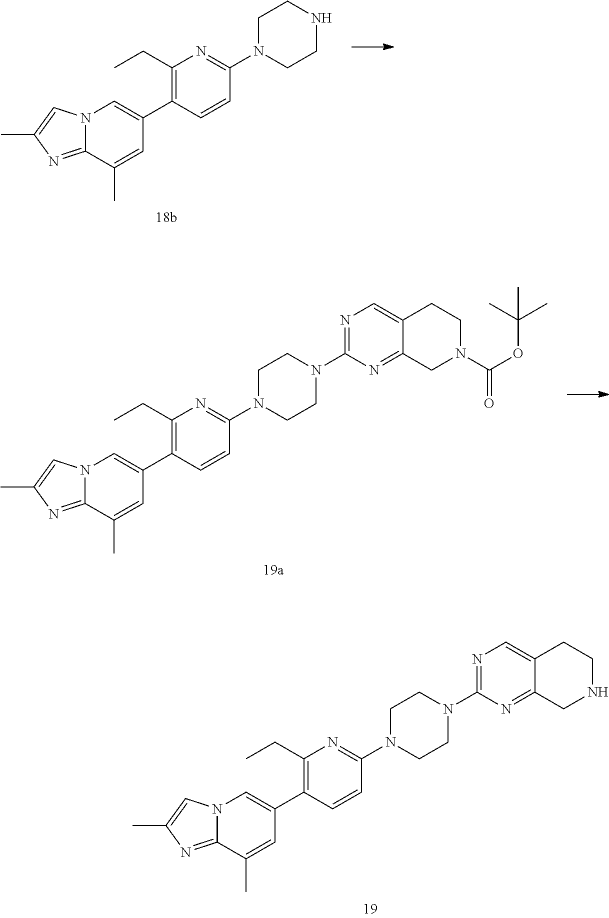

### Step 1: preparation of tert-butyl 2-[4-[5-(2,8-dimethylimidazo[1,2-a]pyridin-6-yl)-6-ethyl-2-pyridyl]piperazin-1-yl]-6,8-dihydro-5H-pyrido[3,4-d]pyrimidine-7-carboxylate

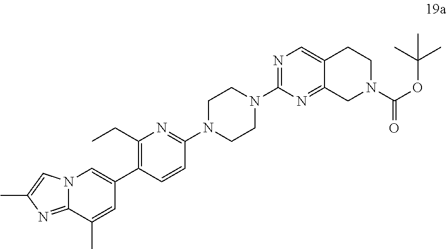 [SMILES: CCc1nc(N2CCN(c3ncc4c(n3)CN(C(=O)OC(C)(C)C)CC4)CC2)ccc1-c1cc(C)c2nc(C)cn2c1]

A mixture of 6-(2-ethyl-6-piperazin-1-yl-3-pyridyl)-2,8-dimethyl-imidazo[1,2-a]pyridine (39.0 mg, 0.116 mmol), tert-butyl 2-chloro-6,8-dihydro-5H-pyrido[3,4-d]pyrimidine-7-carboxylate (47.0 mg, 0.174 mmol, CAS No. 1196156-15-6, vendor: Bide Pharmatech, catalog: BD259018), RuPhos Pd G2 (13.6 mg, 0.017 mmol) and cesium carbonate (113 mg, 0.349 mmol) in 1,4-dioxane (1 mL) was stirred at 110° C. under argon atmosphere for 16 hours. The reaction mixture was concentrated in vacuo and the residue was then purified by flash column (silica gel) eluting with 0-40% MeOH in DCM to afford the crude of tert-butyl 2-[4-[5-(2,8-dimethylimidazo[1,2-a]pyridin-6-yl)-6-ethyl-2-pyridyl]piperazin-1-yl]-6,8-dihydro-5H-pyrido[3,4-d]pyrimidine-7-carboxylate as a yellow oil, which was used in the next step without further purification. MS: calc'd 569 (M+H⁺), measured 569 (M+H⁺).

### Step 2: preparation of 2-[4-[5-(2,8-dimethylimidazo[1,2-a]pyridin-6-yl)-6-ethyl-2-pyridyl]piperazin-1-yl]-5,6,7,8-tetrahydropyrido[3,4-d]pyrimidine

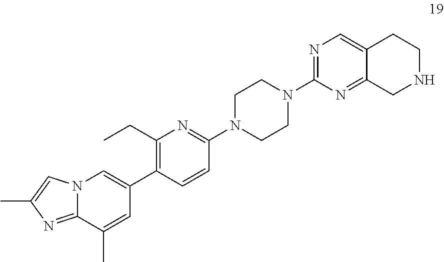 [SMILES: CCc1nc(N2CCN(c3ncc4c(n3)CNCC4)CC2)ccc1-c1cc(C)c2nc(C)cn2c1]

To a solution of tert-butyl 2-[4-[5-(2,8-dimethylimidazo[1,2-a]pyridin-6-yl)-6-ethyl-2-pyridyl]piperazin-1-yl]-6,8-dihydro-5H-pyrido[3,4-d]pyrimidine-7-carboxylate in DCM (3 mL) was added TFA (1.33 g, 0.900 mL, 11.6 mmol) and the reaction mixture was stirred at room temperature under argon atmosphere for 16 hours.

The mixture was concentrated in vacuo and the residue was purified by Prep-HPLC to give 2-[4-[5-(2,8-dimethylimidazo[1,2-a]pyridin-6-yl)-6-ethyl-2-pyridyl]piperazin-1-yl]-5,6,7,8-tetrahydropyrido[3,4-d]pyrimidine (39.4 mg, 58.30% yield over 2 steps) as a light yellow powder. ¹H NMR (400 MHz, CD₃OD) δ 8.62 (s, 1H), 8.32 (s, 1H), 8.00-7.97 (m, 1H), 7.81 (d, J=9.2 Hz, 1H), 7.70 (s, 1H), 7.18 (d, J=9.2 Hz, 1H), 4.23 (s, 2H), 4.03 (dd, J=6.4, 4.0 Hz, 4H), 3.95-3.79 (m, 4H), 3.52 (t, J=6.2 Hz, 2H), 2.97 (t, J=6.1 Hz, 2H), 2.83 (q, J=7.5 Hz, 2H), 2.68 (s, 3H), 2.60 (s, 3H), 1.23 (t, J=7.5 Hz, 3H). MS: calc'd 469 (M+H⁺), measured 469 (M+H⁺).

### Example 20

### 1-[5-(2,8-dimethylimidazo[1,2-a]pyridin-6-yl)-6-ethyl-2-pyridyl]-4-methyl-piperidin-4-amine

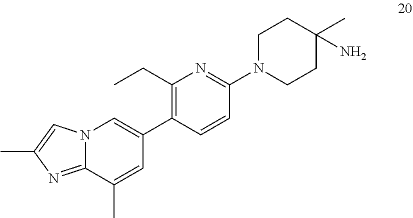 [SMILES: CCc1nc(N2CCC(C)(N)CC2)ccc1-c1cc(C)c2nc(C)cn2c1]

The title compound was prepared in analogy to the preparation of Example 1 by using 6-(6-chloro-2-ethylpyridin-3-yl)-2,8-dimethylimidazo[1,2-a]pyridine and tert-butyl N-(4-methylpiperidin-4-yl)carbamate (CAS No. 163271-08-7, vendor: Pharmablock, catalog: PB02909) instead of 6-(6-chloro-2-isopropylpyridin-3-yl)-8-methylimidazo[1,2-a]pyridine and tert-butyl piperidin-4-ylcarbamate in step 2.

Example 20 (4.0 mg) was obtained as an off-white powder. ¹H NMR (400 MHz, CD₃OD) δ 8.08 (s, 1H), 7.57 (s, 1H), 7.39 (d, J=8.6 Hz, 1H), 6.98 (s, 1H), 6.70 (d, J=8.6 Hz, 1H), 3.91-3.77 (m, 2H), 3.62-3.45 (m, 2H), 2.65 (q, J=7.5 Hz, 2H), 2.55 (s, 3H), 2.43 (s, 3H), 1.75-1.60 (m, 4H), 1.28 (s, 3H), 1.18 (t, J=7.5 Hz, 3H). MS: calc'd 364 (M+H⁺), measured 364 (M+H⁺).

### Example 21

### 6-[2-ethyl-6-(4-piperazin-1-yl-1-piperidyl)-3-pyridyl]-2,8-dimethyl-imidazo[1,2-a]pyridine

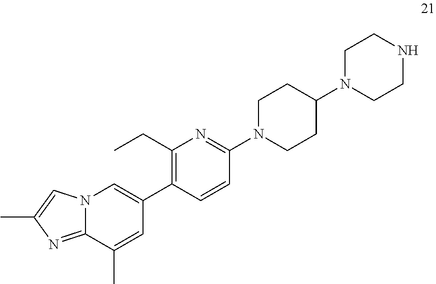 [SMILES: CCc1nc(N2CCC(N3CCNCC3)CC2)ccc1-c1cc(C)c2nc(C)cn2c1]

The title compound was prepared in analogy to the preparation of Example 1 by using 6-(6-chloro-2-ethylpyridin-3-yl)-2,8-dimethylimidazo[1,2-a]pyridine and tert-butyl 4-(piperidin-4-yl)piperazine-1-carboxylate instead of 6-(6-chloro-2-isopropylpyridin-3-yl)-8-methylimidazo[1,2-a]pyridine and tert-butyl piperidin-4-ylcarbamate in step 2.

Example 21 (31 mg) was obtained as a yellow solid. ¹H NMR (400 MHz, CD₃OD, 298 K) δ (ppm)=1.21 (t, J=7.52 Hz, 3H), 1.73-1.87 (m, 2H), 2.21 (br d, J=11.13 Hz, 2H), 2.59 (d, J=0.86 Hz, 3H), 2.67 (s, 3H), 2.70-2.80 (m, 2H), 3.12 (br t, J=11.98 Hz, 2H), 3.37-3.48 (m, 5H), 3.49-3.56 (m, 4H), 4.58 (br d, J=13.69 Hz, 2H), 7.01 (d, J=9.05 Hz, 1H), 7.62-7.70 (m, 2H), 7.96 (d, J=1.10 Hz, 1H), 8.52-8.59 (m, 1H). MS: calc'd 419 (M+H⁺), measured 419 (M+H⁺).

### Example 22

### 3-[3-ethyl-5-(8-methylimidazo[1,2-a]pyridin-6-yl)-2-pyridyl]-3,9-diazaspiro[5.5]undecane

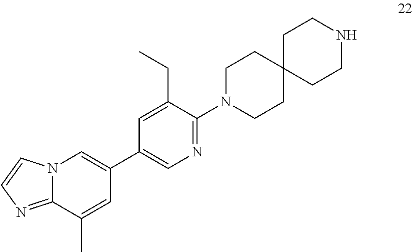 [SMILES: CCc1cc(-c2cc(C)c3nccn3c2)cnc1N1CCC2(CCNCC2)CC1]

### Step 1: preparation of 6-(6-chloro-5-ethyl-3-pyridyl)-8-methyl-imidazo[1,2-a]pyridine

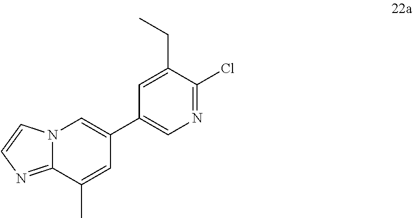 [SMILES: CCc1cc(-c2cc(C)c3nccn3c2)cnc1Cl]

The title compound was prepared in analogy to the preparation of compound 1a by 2-chloro-3-ethyl-5-(4,4,5,5-tetramethyl-1,3,2-dioxaborolan-2-yl)pyridine (Int-B7) instead of 6-chloro-2-isopropyl-3-(4,4,5,5-tetramethyl-1,3,2-dioxaborolan-2-yl)pyridine (Int-B2) in Step 1.

### Step 2: preparation of 3-[3-ethyl-5-(8-methylimidazo[1,2-a]pyridin-6-yl)-2-pyridyl]-3,9-diazaspiro[5.5]undecane

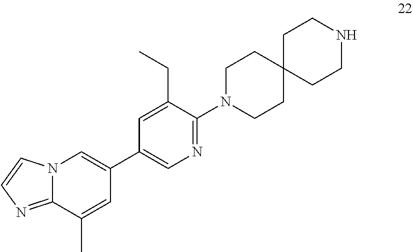 [SMILES: CCc1cc(-c2cc(C)c3nccn3c2)cnc1N1CCC2(CCNCC2)CC1]

The title compound was prepared in analogy to the preparation of Example 1 by using 6-(6-chloro-5-ethyl-3-pyridyl)-8-methyl-imidazo[1,2-a]pyridine and tert-butyl 3,9-diazaspiro[5.5]undecane-3-carboxylate instead of 6-(6-chloro-2-isopropylpyridin-3-yl)-8-methylimidazo[1,2-a]pyridine and tert-butyl N-(4-piperidyl)carbamate (CAS No. 73874-95-0, vendor: Accela ChemBio, catalog SY002020) in Step 2.

Example 22 (2 mg) was obtained as a yellow solid. ¹H NMR (400 MHz, CD₃OD, 298 K) δ (ppm)=8.98-8.96 (m, 1H), 8.45 (d, J=2.4 Hz, 1H), 8.24 (d, J=2.2 Hz, 1H), 8.10-8.06 (m, 2H), 8.02 (d, J=2.4 Hz, 1H), 3.27-3.19 (m, 8H), 2.83-2.75 (m, 2H), 2.73 (s, 3H), 1.81 (td, J=5.8, 13.6 Hz, 8H), 1.36 (t, J=7.5 Hz, 3H). MS: calc'd 390 (M+H⁺), measured 390 (M+H⁺).

### Example 23

### N,N-dimethyl-3-(8-methylimidazo[1,2-a]pyridin-6-yl)-6-(4-piperazin-1-yl-1-piperidyl)pyridin-2-amine

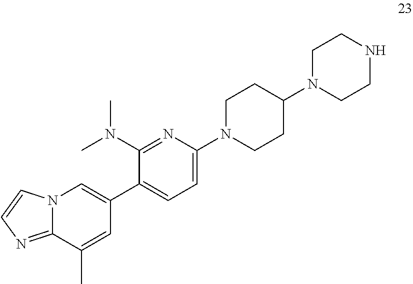 [SMILES: Cc1cc(-c2ccc(N3CCC(N4CCNCC4)CC3)nc2N(C)C)cn2ccnc12]

The title compound was prepared according to the following scheme:

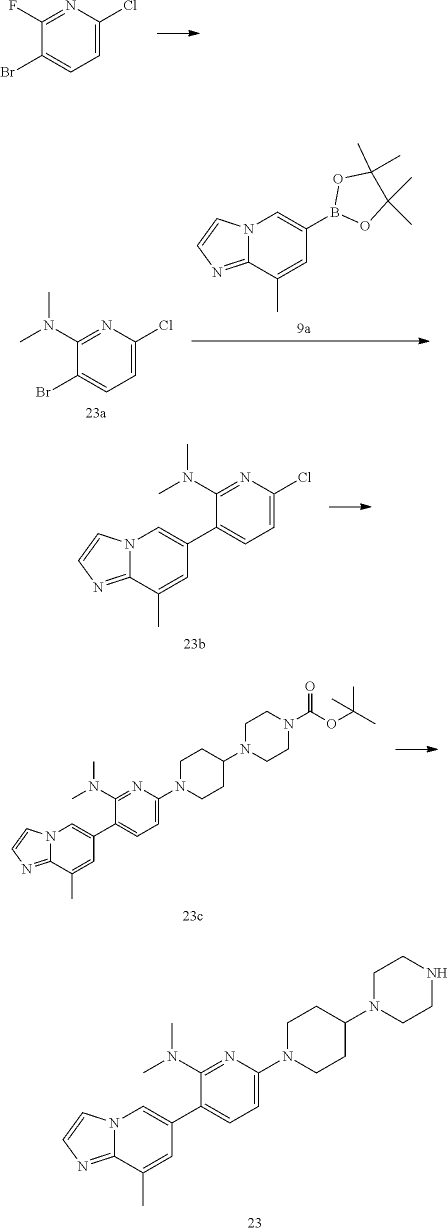 [SMILES: Fc1nc(Cl)ccc1Br ; CN(C)c1nc(Cl)ccc1Br ; Cc1cc(B2OC(C)(C)C(C)(C)O2)cn2ccnc12 ; Cc1cc(-c2ccc(Cl)nc2N(C)C)cn2ccnc12 ; Cc1cc(-c2ccc(N3CCC(N4CCN(C(=O)OC(C)(C)C)CC4)CC3)nc2N(C)C)cn2ccnc12 ; Cc1cc(-c2ccc(N3CCC(N4CCNCC4)CC3)nc2N(C)C)cn2ccnc12]

### Step1: preparation of 3-bromo-6-chloro-N,N-dimethyl-pyridin-2-amine

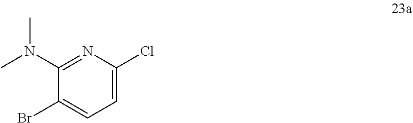

A mixture of 3-bromo-6-chloro-2-fluoro-pyridine (1300 mg, 6.18 mmol), dimethylamine hydrochloride (554.11 mg, 6.8 mmol) and K₂CO₃ (853.84 mg, 6.18 mmol) in DMF (10 mL) was stirred at 100° C. overnight. After the reaction was completed, the mixture was diluted with water (30 mL) and the resulting mixture was extracted with EtOAc (30 mL) three times. The combined organic layer was then washed with brine, dried over Na₂SO₄ and concentrated in vacuo. The residue was then purified by flash column eluting with a gradient of EA/PE (0% to 50%) to give 3-bromo-6-chloro-N,N-dimethyl-pyridin-2-amine (1000 mg, 68.73%) as colorless oil. MS: calc'd 235 (M+H⁺), measured 235 (M+H⁺).

### Step2: preparation of [6-chloro-3-(8-methylimidazo[1,2-a]pyridin-6-yl)-2-pyridyl]-dimethyl-amine

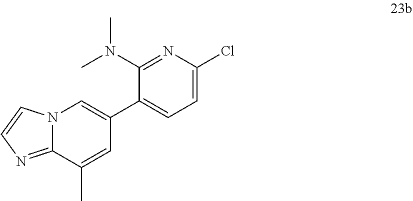 [SMILES: Cc1cc(-c2ccc(Cl)nc2N(C)C)cn2ccnc12]

A mixture of 8-methyl-6-(4,4,5,5-tetramethyl-1,3,2-dioxaborolan-2-yl)imidazo[1,2-a]pyridine (200 mg, 0.775 mmol), 3-bromo-6-chloro-N,N-dimethyl-pyridin-2-amine (182.48 mg, 0.775 mmol), 2 M K₂CO₃ (0.5 mL, 1 mmol) and PdCl₂(dppf)-CH₂Cl₂ adduct (128.11 mg, 0.155 mmol) in 1,4-dioxane (5 mL) was stirred at 100° C. under N₂ atmosphere for 2 hours. After the reaction was completed, the mixture was then concentrated in vacuo and the residue as then purified by flash column eluting with a gradient of EA/PE (0% to 50%) to give [6-chloro-3-(8-methylimidazo[1,2-a]pyridin-6-yl)-2-pyridyl]-dimethyl-amine (200 mg, 90.01% yield) as brown solid. MS: calc'd 287 (M+H⁺), measured 287 (M+H⁺).

### Step 3: preparation of tert-butyl 4-[1-[6-(dimethylamino)-5-(8-methylimidazo[1,2-a]pyridin-6-yl)-2-pyridyl]-4-piperidyl]piperazine-1-carboxylate

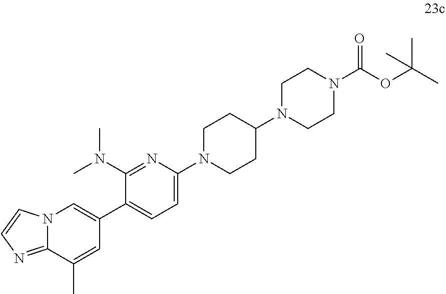 [SMILES: Cc1cc(-c2ccc(N3CCC(N4CCN(C(=O)OC(C)(C)C)CC4)CC3)nc2N(C)C)cn2ccnc12]

To a mixture of [6-chloro-3-(8-methylimidazo[1,2-a]pyridin-6-yl)-2-pyridyl]-dimethyl-amine (45.4 mg, 0.158 mmol), 4-(4-piperidyl)piperazine-1-carboxylic acid tert-butyl ester (127.95 mg, 0.475 mmol, CAS No. 205059-24-1, vendor: Bide Pharmatech, catalog BD57121), Cs₂CO₃ (51.6 mg, 158 μmol) in dioxane (15 mL) was added Ruphos Pd G2 (50 mg, 158 μmol) and the mixture was then stirred at 100° C. overnight. After the reaction was completed, the mixture was then concentrated in vacuo, the residue was then purified by flash column eluting with a gradient of MeOH/DCM (0% to 15%) to give tert-butyl 4-[1-[6-(dimethylamino)-5-(8-methylimidazo[1,2-a]pyridin-6-yl)-2-pyridyl]-4-piperidyl]piperazine-1-carboxylate (40 mg, 48.61%) as dark brown solid. MS: calc'd 520 (M+H⁺), measured 520 (M+H⁺).

### Step 4: preparation of N,N-dimethyl-3-(8-methylimidazo[1,2-a]pyridin-6-yl)-6-(4-piperazin-1-yl-1-piperidyl)pyridin-2-amine

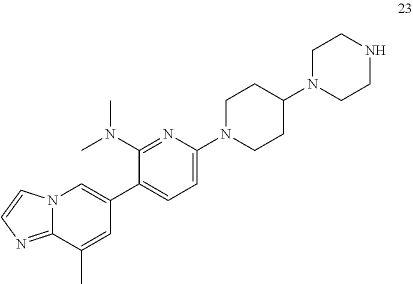 [SMILES: Cc1cc(-c2ccc(N3CCC(N4CCNCC4)CC3)nc2N(C)C)cn2ccnc12]

To a solution of tert-butyl 4-[1-[6-(dimethylamino)-5-(8-methylimidazo[1,2-a]pyridin-6-yl)-2-pyridyl]-4-piperidyl]piperazine-1-carboxylate (30 mg, 0.058 mmol) in DCM (10 mL) was added TFA (0.3 mL) and the mixture was then stirred at room temperature overnight. After the reaction was completed, the mixture was concentrated in vacuo and the residue was then purified by Prep-HPLC to give dimethyl-[3-(8-methylimidazo[1,2-a]pyridin-6-yl)-6-(4-piperazinopiperidino)-2-pyridyl]amine (4 mg, 15.19%) as off-white powder. ¹H NMR (400 MHz, METHANOL-d₄) δ=8.24-8.12 (m, 1H), 7.71 (d, J=1.2 Hz, 1H), 7.48-7.41 (m, 1H), 7.28 (d, J=8.3 Hz, 1H), 7.16 (s, 1H), 6.23 (d, J=8.3 Hz, 1H), 4.40-4.31 (m, 2H), 2.92-2.81 (m, 4H), 2.77-2.69 (m, 2H), 2.64-2.60 (s, 6H), 2.59-2.55 (m, 4H), 2.48-2.44 (s, 3H), 2.43-2.35 (m, 1H), 1.94-1.85 (m, 2H), 1.49-1.38 (m, 2H). MS: calc'd 420 (M+H⁺), measured 420 (M+H⁺).

### Example 24

### 6-[4-[(3R,4R)-3-amino-4-methoxy-pyrrolidin-1-yl]-1-piperidyl]-N,N-dimethyl-3-(8-methylimidazo[1,2-a]pyridin-6-yl)pyridin-2-amine

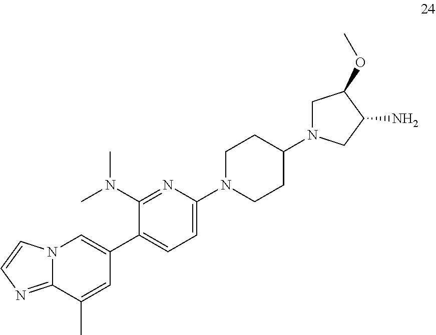 [SMILES: CO[C@@H]1CN(C2CCN(c3ccc(-c4cc(C)c5nccn5c4)c(N(C)C)n3)CC2)C[C@H]1N]

### Step 1: preparation of tert-butyl N-[(3R,4R)-1-(1-benzyl-4-piperidyl)-4-methoxy-pyrrolidin-3-yl]carbamate

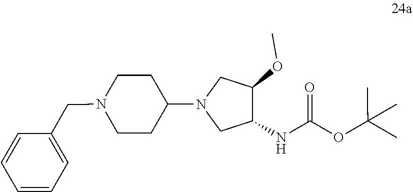 [SMILES: CO[C@@H]1CN(C2CCN(Cc3ccccc3)CC2)C[C@H]1NC(=O)OC(C)(C)C]

To a mixture of 1-benzylpiperidin-4-one (525 mg, 2.77 mmol, CAS No. 3612-20-2, vendor: Bide Pharmatech), tert-butyl N-[(3S,4S)-4-methoxypyrrolidin-3-yl]carbamate (500 mg, 2.31 mmol, 128739-92-4, vendor: PharmaBlock Sciences (Nanjing), Inc., catalog PBN20121069), AcOH (14.0 mg, 0.23 mmol) in DCM (25 mL) was added NaBH(OAc)₃ (2.43 g, 11.5 mmol) and the mixture was stirred at 40° C. for 6 hours. After the reaction was completed, the mixture was then concentrated in vacuo. The residue was then purified by flash column eluting with a gradient of MeOH/DCM (0% to 20%) to give tert-butyl N-[(3R,4R)-1-(1-benzyl-4-piperidyl)-4-methoxy-pyrrolidin-3-yl]carbamate (800 mg, 88.9% yield) as a yellow oil. MS: calc'd 390 (M+H⁺), measured 390 (M+H⁺).

### Step 2: preparation of tert-butyl N-[(3R,4R)-4-methoxy-1-(4-piperidyl)pyrrolidin-3-yl]carbamate

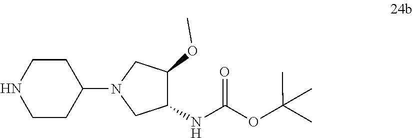 [SMILES: CO[C@@H]1CN(C2CCNCC2)C[C@H]1NC(=O)OC(C)(C)C]

To a solution of tert-butyl N-[(3R,4R)-1-(1-benzyl-4-piperidyl)-4-methoxy-pyrrolidin-3-yl]carbamate (800 mg, 2.1 mmol) in MeOH (10 mL) was added palladium on carbon (10 weight % loading, 50 mg) and the mixture was then stirred under H₂ atmosphere at 25° C. for 6 hours. After the reaction was completed, the mixture was then concentrated in vacuo to give the crude tert-butyl N-[(3R,4R)-4-methoxy-1-(4-piperidyl)pyrrolidin-3-yl]carbamate (500 mg, 80.3% yield) as a brown solid. MS: calc'd 300 (M+H⁺), measured 300 (M+H⁺).

### Step 3: preparation of 6-[4-[(3R,4R)-3-amino-4-methoxy-pyrrolidin-1-yl]-1-piperidyl]-N,N-dimethyl-3-(8-methylimidazo[1,2-a]pyridin-6-yl)pyridin-2-amine

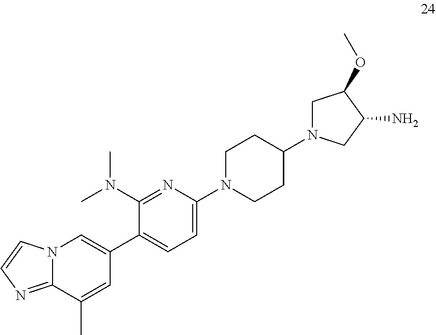 [SMILES: CO[C@@H]1CN(C2CCN(c3ccc(-c4cc(C)c5nccn5c4)c(N(C)C)n3)CC2)C[C@H]1N]

The title compound was prepared in analogy to the preparation of Example 23 by using tert-butyl N-[(3R,4R)-4-methoxy-1-(4-piperidyl)pyrrolidin-3-yl]carbamate instead of compound 4-(4-piperidyl)piperazine-1-carboxylic acid tert-butyl ester in Step 3.

Example 24 (1.5 mg) was obtained as an off-white powder. ¹H NMR (400 MHz, METHANOL-d₄) δ (ppm)=8.21 (d, J=0.9 Hz, 1H), 7.71 (d, J=1.3 Hz, 1H), 7.43 (d, J=1.2 Hz, 1H), 7.28 (d, J=8.3 Hz, 1H), 7.20-7.13 (m, 1H), 6.29-6.18 (m, 1H), 4.48 (br s, 2H), 4.27 (br d, J=13.3 Hz, 2H), 3.26 (s, 3H), 2.99-2.91 (m, 2H), 2.84-2.74 (m, 2H), 2.69-2.65 (m, 1H), 2.62 (s, 6H), 2.45 (s, 3H), 2.40-2.33 (m, 1H), 2.32-2.26 (m, 1H), 1.91-1.82 (m, 2H), 1.47-1.35 (m, 2H). MS: calc'd 450 (M+H⁺), measured 450 (M+H⁺).

### Example 25

### 6-[2-methoxy-6-(4-piperazin-1-yl-1-piperidyl)-3-pyridyl]-8-methyl-imidazo[1,2-a]pyridine

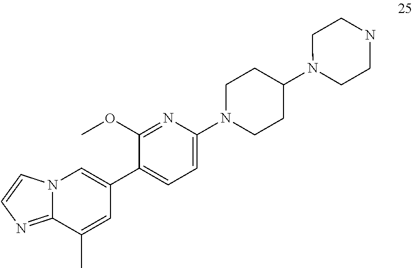 [SMILES: COc1nc(N2CCC(N3CCNCC3)CC2)ccc1-c1cc(C)c2nccn2c1]

The title compound was prepared in analogy to the preparation of Example 23 by using 3-bromo-6-chloro-2-methoxy-pyridine instead of 3-bromo-6-chloro-N,N-dimethyl-pyridin-2-amine in Step 2.

Example 25 (12 mg, 17.9% yield) was obtained as a yellow solid. ¹H NMR (400 MHz, CD₃OD, 298 K) δ (ppm)=8.86-8.84 (m, 1H), 8.20 (d, J=2.1 Hz, 1H), 8.03-7.99 (m, 2H), 7.73 (d, J=8.4 Hz, 1H), 6.52 (d, J=8.6 Hz, 1H), 4.61-4.54 (m, 2H), 3.97 (s, 3H), 3.44-3.37 (m, 4H), 3.25-3.19 (m, 4H), 3.17-3.08 (m, 1H), 3.02-2.91 (m, 2H), 2.67 (s, 3H), 2.09 (br d, J=12.3 Hz, 2H), 1.73-1.61 (m, 2H). MS: calc'd 407 (M+H⁺), measured 407 (M+H⁺).

### Example 26

### (3R,4R)-1-[1-[4-ethyl-5-(8-methylimidazo[1,2-a]pyridin-6-yl)pyrimidin-2-yl]-4-piperidyl]-4-methoxy-pyrrolidin-3-amine

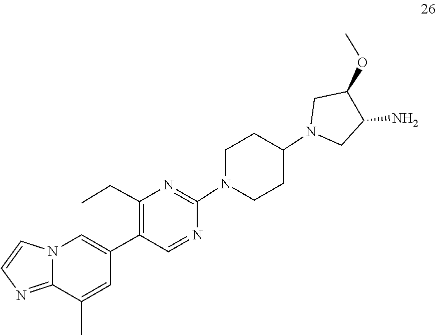 [SMILES: CCc1nc(N2CCC(N3C[C@@H](N)[C@H](OC)C3)CC2)ncc1-c1cc(C)c2nccn2c1]

The title compound was prepared according to the following scheme:

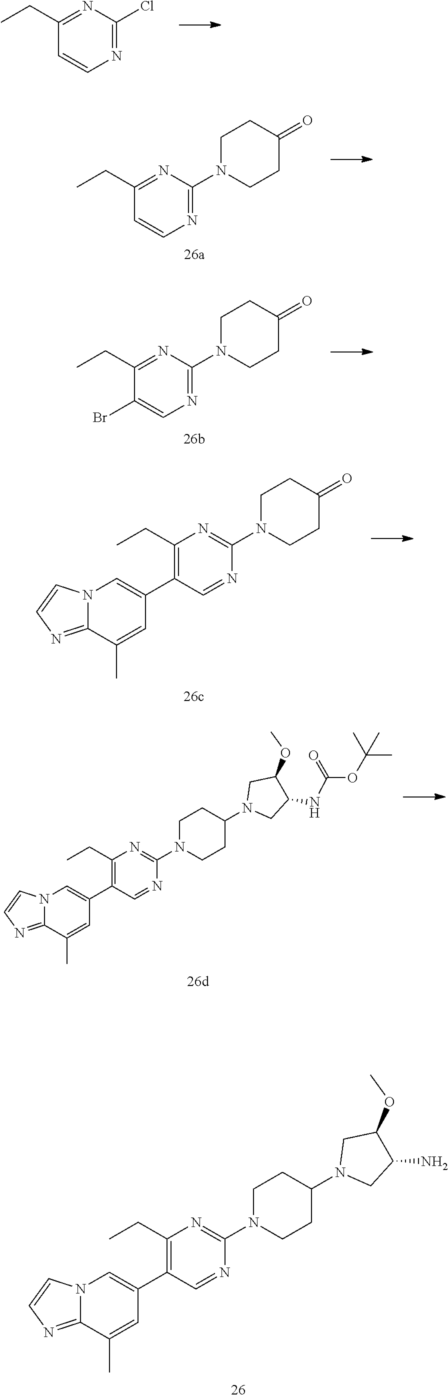

### Step 1: preparation of 1-(4-ethylpyrimidin-2-yl)piperidin-4-one

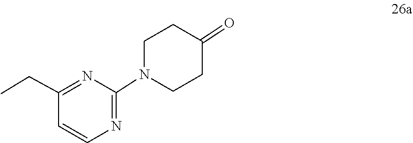

To a solution of 2-chloro-4-ethylpyrimidine (1.43 g, 10 mmol, CAS No. 188707-99-5, vendor: Accela ChemBio Inc, catalog: SY021045) and piperidin-4-one hydrochloride (2.03 g, 15 mmol) in DMF (15 mL) was added K₂CO₃ (4.84 g, 35 mmol) and the mixture was stirred at 130° C. under argon atmosphere for 16 hours. After the reaction was completed, the reaction mixture was filtered through a pad of Celite, and then concentrated in vacuo. The residue was purified by flash chromatography eluting with 0-50% EA/PE to give 1-(4-ethylpyrimidin-2-yl)piperidin-4-one (1.62 g, 78.92%) as colorless oil. MS: calc'd 206 (M+H⁺), measured 206 (M+H⁺).

### Step 2: preparation of 1-(5-bromo-4-ethyl-pyrimidin-2-yl)piperidin-4-one

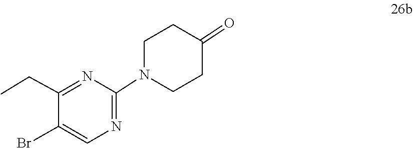 [SMILES: CCc1nc(N2CCC(=O)CC2)ncc1Br]

To a solution of 1-(4-ethylpyrimidin-2-yl)piperidin-4-one (522 mg, 2.54 mmol) in DCM (7 mL) at 0° C. was added silica gel (5 mg) and NBS (498 mg, 2.80 mmol). The reaction mixture was stirred at room temperature in dark under argon atmosphere for 16 hours.

The mixture was concentrated in vacuo, and the residue was purified by flash chromatography eluting with 0-30% EA/PE to afford 1-(5-bromo-4-ethyl-pyrimidin-2-yl)piperidin-4-one (464 mg, 64.23% yield) as a colorless oil. MS: calc'd 284 (M+H⁺), measured 284 (M+H⁺).

### Step 3: preparation of 1-[4-ethyl-5-(8-methylimidazo[1,2-a]pyridin-6-yl)pyrimidin-2-yl]piperidin-4-one

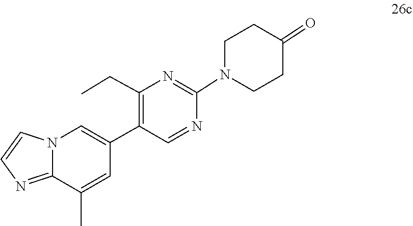 [SMILES: CCc1nc(N2CCC(=O)CC2)ncc1-c1cc(C)c2nccn2c1]

To a solution of 8-methyl-6-(4,4,5,5-tetramethyl-1,3,2-dioxaborolan-2-yl)imidazo[1,2-a]pyridine (9a, 258 mg, 1 mmol) and 1-(5-bromo-4-ethyl-pyrimidin-2-yl)piperidin-4-one (341 mg, 1.2 mmol) in dioxane (4 mL) was added [1,1′-bis(diphenylphosphino)ferrocene]dichloropalladium(II) (73 mg, 0.1 mmol) and potassium carbonate (276 mg, 2 mmol). The reaction mixture was stirred at 80° C. under argon atmosphere for 16 hours.

The mixture was concentrated in vacuo, and the residue was diluted with DCM and filtered through a pad of Celite. The filtrate was concentrated in vacuo and the residue was purified by flash chromatography eluting with 0-60% EtOAc (containing 10% MeOH) in petroleum ether to give 1-[4-ethyl-5-(8-methylimidazo[1,2-a]pyridin-6-yl)pyrimidin-2-yl]piperidin-4-one (161 mg, 48.0% yield) as a light yellow solid. MS: calc'd 336 (M+H⁺), measured 336 (M+H⁺).

### Step 4: preparation of tert-butyl N-[(3R,4R)-1-[1-[4-ethyl-5-(8-methylimidazo[1,2-a]pyridin-6-yl)pyrimidin-2-yl]-4-piperidyl]-4-methoxy-pyrrolidin-3-yl]carbamate

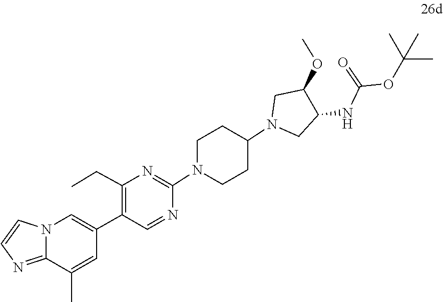 [SMILES: CCc1nc(N2CCC(N3C[C@@H](NC(=O)OC(C)(C)C)[C@H](OC)C3)CC2)ncc1-c1cc(C)c2nccn2c1]

To a solution of 1-[4-ethyl-5-(8-methylimidazo[1,2-a]pyridin-6-yl)pyrimidin-2-yl]piperidin-4-one (43.6 mg, 0.130 mmol) and tert-butyl N-[(3R,4R)-4-methoxypyrrolidin-3-yl]carbamate (42.2 mg, 0.195 mmol) in EtOH (1.3 mL) was added acetic acid (15.6 mg, 7.54 μL, 0.260 mmol) and the reaction mixture was stirred at 50° C. for 1 hour. Then to the resulting mixture was added sodium cyanoborohydride (16.3 mg, 0.260 mmol) and the mixture was further stirred at 50° C. under argon atmosphere for 16 hours. After the reaction was completed, the mixture was then concentrated in vacuo and the residue was purified by flash column eluting with 0-60% MeOH in DCM to afford the crude tert-butyl N-[(3R,4R)-1-[1-[4-ethyl-5-(8-methylimidazo[1,2-a]pyridin-6-yl)pyrimidin-2-yl]-4-piperidyl]-4-methoxy-pyrrolidin-3-yl]carbamate as a yellow oil, which was used in the next step without further purification. MS: calc'd 536 (M+H⁺), measured 536 (M+H⁺).

### Step 5: preparation of (3R, 4R)-1-[1-[4-ethyl-5-(8-methylimidazo[1,2-a]pyridin-6-yl)pyrimidin-2-yl]-4-piperidyl]-4-methoxy-pyrrolidin-3-amine

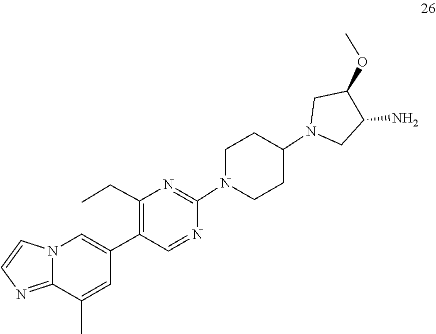 [SMILES: CCc1nc(N2CCC(N3C[C@@H](N)[C@H](OC)C3)CC2)ncc1-c1cc(C)c2nccn2c1]

To a solution of tert-butyl N-[(3R,4R)-1-[1-[4-ethyl-5-(8-methylimidazo[1,2-a]pyridin-6-yl)pyrimidin-2-yl]-4-piperidyl]-4-methoxy-pyrrolidin-3-yl]carbamate from the previous step in DCM (2.6 mL) was added TFA (1.48 g, 0.99 mL, 13.0 mmol), and the reaction mixture was stirred at room temperature under argon atmosphere for 16 hours. The mixture was concentrated in vacuo, and the residue was purified by prep-HPLC to give (3R,4R)-1-[1-[4-ethyl-5-(8-methylimidazo[1,2-a]pyridin-6-yl)pyrimidin-2-yl]-4-piperidyl]-4-methoxy-pyrrolidin-3-amine (20.3 mg, 28.41% yield over 2 steps) as a white powder. ¹H NMR (400 MHz, CD₃OD) δ 8.65 (s, 1H), 8.25-8.22 (m, 2H), 8.09 (d, J=2.2 Hz, 1H), 7.75 (s, 1H), 5.07 (d, J=13.8 Hz, 2H), 4.32-4.19 (m, 1H), 4.11 (dd, J=8.2, 12.5 Hz, 1H), 4.06-3.95 (m, 1H), 3.78 (s, 2H), 3.67-3.52 (m, 2H), 3.46 (s, 3H), 3.00 (t, J=12.1 Hz, 2H), 2.74-2.62 (m, 5H), 2.26 (s, 2H), 1.79-1.64 (m, 2H), 1.22 (t, J=7.5 Hz, 3H). MS: calc'd 436 (M+H⁺), measured 436 (M+H⁺).

### Example 27

### 6-[4-ethyl-2-(4-piperazin-1-yl-1-piperidyl)pyrimidin-5-yl]-8-methyl-imidazo[1,2-a]pyridine

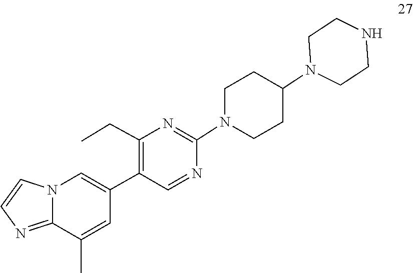 [SMILES: CCc1nc(N2CCC(N3CCNCC3)CC2)ncc1-c1cc(C)c2nccn2c1]

The title compound was prepared in analogy to the preparation of Example 26 by using tert-butyl piperazine-1-carboxylate instead of tert-butyl N-[(3R,4R)-4-methoxypyrrolidin-3-yl]carbamate in Step 4. 6-[4-ethyl-2-(4-piperazin-1-yl-1-piperidyl)pyrimidin-5-yl]-8-methyl-imidazo[1,2-a]pyridine (8.3 mg) was obtained as a white powder. ¹H NMR (400 MHz, CD₃OD, 298 K) δ 8.66-8.63 (m, 1H), 8.25-8.21 (m, 2H), 8.09 (d, J=2.2 Hz, 1H), 7.75 (s, 1H), 5.07 (d, J=13.6 Hz, 2H), 3.59-3.40 (m, 9H), 3.02 (t, J=11.9 Hz, 2H), 2.72-2.65 (m, 5H), 2.20 (d, J=11.5 Hz, 2H), 1.69 (dd, J=4.1, 12.2 Hz, 2H), 1.22 (t, J=7.5 Hz, 3H). MS: calc'd 406 (M+H⁺), measured 406 (M+H⁺).

### Example 28

### 2-[1-[4-ethyl-5-(8-methylimidazo[1,2-a]pyridin-6-yl)pyrimidin-2-yl]-4-piperidyl]-5-oxa-2,8-diazaspiro[3.5]nonane

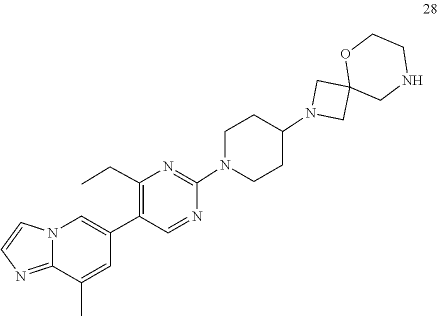 [SMILES: CCc1nc(N2CCC(N3CC4(CNCCO4)C3)CC2)ncc1-c1cc(C)c2nccn2c1]

The title compound was prepared in analogy to the preparation of Example 26 by using of tert-butyl 5-oxa-2,8-diazaspiro[3.5]nonane-8-carboxylate instead of tert-butyl N-[(3R,4R)-4-methoxypyrrolidin-3-yl]carbamate in Step 4. 2-[1-[4-ethyl-5-(8-methylimidazo[1,2-a]pyridin-6-yl)pyrimidin-2-yl]-4-piperidyl]-5-oxa-2,8-diazaspiro[3.5]nonane (28.7 mg) was obtained as a white powder. ¹H NMR (400 MHz, CD₃OD) δ 8.65 (s, 1H), 8.26-8.20 (m, 2H), 8.09 (d, J=2.2 Hz, 1H), 7.75 (s, 1H), 5.05 (br d, J=13.7 Hz, 2H), 4.45 (br d, J=11.5 Hz, 2H), 4.40-4.31 (m, 2H), 4.12-3.95 (m, 2H), 3.66-3.50 (m, 3H), 3.30-3.26 (m, 2H), 3.00 (br t, J=12.0 Hz, 2H), 2.74-2.64 (m, 5H), 2.13 (br d, J=10.9 Hz, 2H), 1.47 (dq, J=4.2, 12.1 Hz, 2H), 1.21 (t, J=7.5 Hz, 3H). MS: calc'd 448 (M+H⁺), measured 448 (M+H⁺).

### Example 29

The following tests were carried out in order to determine the activity of the compounds of formula (I) in HEK293-Blue-hTLR-7/8/9 cells assay.

HEK293-Blue-hTLR-7 Cells Assay:

A stable HEK293-Blue-hTLR-7 cell line was purchased from InvivoGen (Cat. #: hkb-htlr7, San Diego, Calif., USA). These cells were originally designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was declined by TLR7 antagonist under the stimulation of a ligand, such as R848 (Resiquimod), for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR7 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620-655 nm using a spectrophotometer. The signaling pathway that TLR7 activation leads to downstream NF-κB activation has been widely accepted, and therefore similar reporter assay was modified for evaluating TLR7 antagonist.

HEK293-Blue-hTLR-8 Cells Assay:

A stable HEK293-Blue-hTLR-8 cell line was purchased from InvivoGen (Cat. #: hkb-htlr8, San Diego, Calif., USA). These cells were originally designed for studying the stimulation of human TLR8 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR8 cells with TLR8 ligands. Therefore the reporter expression was declined by TLR8 antagonist under the stimulation of a ligand, such as R848, for incubation of 20 hrs. The cell culture supernatant SEAP reporter activity was determined using QUANTI-Blue™ kit (Cat. #: rep-gb1, Invivogen, San Diego, Ca, USA) at a wavelength of 640 nm, a detection medium that turns purple or blue in the presence of alkaline phosphatase.

HEK293-Blue-hTLR8 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 60 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620655 nm using a spectrophotometer. The signaling pathway that TLR8 activation leads to downstream NF-κB activation has been widely accepted, and therefore similar reporter assay was modified for evaluating TLR8 antagonist.

HEK293-Blue-hTLR-9 Cells Assay:

A stable HEK293-Blue-hTLR-9 cell line was purchased from InvivoGen (Cat. #: hkb-htlr9, San Diego, Calif., USA). These cells were originally designed for studying the stimulation of human TLR9 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR9 cells with TLR9 ligands. Therefore the reporter expression was declined by TLR9 antagonist under the stimulation of a ligand, such as ODN2006 (Cat. #: tlr1-2006-1, Invivogen, San Diego, Calif., USA), for incubation of 20 hrs. The cell culture supernatant SEAP reporter activity was determined using QUANTI-Blue™ kit (Cat. #: rep-qb1, Invivogen, San Diego, Calif., USA) at a wavelength of 640 nm, a detection medium that turns purple or blue in the presence of alkaline phosphatase.

HEK293-Blue-hTLR9 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL, of 20 uM ODN2006 in above DMEM, perform incubation under 37° C. in a CO₂ incubator for 20 hrs. Then 20 μL of the supernatant from each well was incubated with 180 μL, Quanti-blue substrate solution at 37° C. for 2 h and the absorbance was read at 620655 nm using a spectrophotometer. The signaling pathway that TLR9 activation leads to downstream NF-κB activation has been widely accepted, and therefore similar reporter assay was modified for evaluating TLR9 antagonist.

The compounds of formula (I) have human TLR7 and/or TLR8 inhibitory activities (IC₅₀ value)<0.1 μM, and human TLR9 inhibitory activity<0.5 μM. Activity data of the compounds of the present invention were shown in Table 1.

TABLE 1
The activity of the compounds of present invention
in HEK293-Blue-hTLR-7/8/9 cells assays

| | HEK/hTLR7 | HEK/hTLR8 | HEK/hTLR9 |
|---|---|---|---|
| Example No | IC₅₀ (μM) | IC₅₀ (μM) | IC₅₀ (μM) |
| 1 | 0.0014 | 0.0013 | 0.292 |
| 2 | 0.0016 | 0.0013 | 0.151 |
| 3 | 0.0058 | 0.0012 | 0.198 |
| 4 | 0.0089 | 0.0029 | 0.154 |
| 5 | 0.0214 | 0.0304 | 0.227 |
| 6 | 0.0136 | 0.027 | 0.269 |
| 7 | 0.0227 | 0.0086 | 0.112 |
| 8 | 0.0067 | 0.0023 | 0.294 |
| 9 | 0.0132 | 0.0107 | 0.169 |
| 10 | 0.0019 | 0.0028 | 0.217 |
| 11 | 0.0163 | 0.0168 | 0.111 |
| 12 | 0.0163 | 0.0011 | 0.145 |
| 13 | 0.0074 | 0.0284 | 0.293 |
| 14 | 0.0039 | 0.0004 | 0.216 |
| 15 | 0.0027 | 0.0003 | 0.286 |
| 16 | 0.0062 | 0.0157 | 0.111 |
| 17 | 0.0173 | 0.001 | 0.224 |
| 18 | 0.0224 | 0.0288 | 0.255 |
| 19 | 0.0435 | 0.0013 | 0.131 |
| 20 | 0.0358 | 0.0418 | 0.108 |
| 21 | 0.0059 | 0.0036 | 0.14 |
| 22 | 0.0033 | 0.04 | 0.132 |
| 23 | 0.0064 | 0.0081 | 0.118 |
| 24 | 0.0201 | 0.004 | 0.107 |
| 25 | 0.0069 | 0.0112 | 0.092 |
| 26 | 0.0074 | 0.001 | 0.229 |
| 27 | 0.0033 | 0.0046 | 0.259 |
| 28 | 0.0153 | 0.0013 | 0.159 |

### Example 30

hERG Channel Inhibition Assay:

The hERG channel inhibition assay is a highly sensitive measurement that identifies compounds exhibiting hERG inhibition related to cardiotoxicity in vivo. The hERG K⁺ channels were cloned in humans and stably expressed in a CHO (Chinese hamster ovary) cell line. CHO_{hERG} cells were used for patch-clamp (voltage-clamp, whole-cell) experiments. Cells were stimulated by a voltage pattern to activate hERG channels and conduct I_{KhERG} currents (rapid delayed outward rectifier potassium current of the hERG channel). After the cells were stabilized for a few minutes, the amplitude and kinetics of I_{KhERG} were recorded at a stimulation frequency of 0.1 Hz (6 bpm). Thereafter, the test compound was added to the preparation at increasing concentrations. For each concentration, an attempt was made to reach a steady-state effect, usually, this was achieved within 3-10 min at which time the next highest concentration was applied. The amplitude and kinetics of I_{KhERG} are recorded in each concentration of the drug which were compared to the control values (taken as 100%). (references: Redfern W S, Carlsson L, Davis A S, Lynch W G, MacKenzie I, Palethorpe S, Siegl P K, Strang I, Sullivan A T, Wallis R, Camm A J, Hammond T G. 2003; Relationships between preclinical cardiac electrophysiology, clinical QT interval prolongation and torsade de pointes for a broad range of drugs: evidence for a provisional safety margin in drug development. Cardiovasc. Res. 58:32-45, Sanguinetti M C, Tristani-Firouzi M. 2006; hERG potassium channels and cardiac arrhythmia. Nature 440:463-469, Webster R, Leishman D, Walker D. 2002; Towards a drug concentration effect relationship for QT prolongation and torsades de pointes. Curr. Opin. Drug Discov. Devel. 5:116-26).

Results of hERG are given in Table 2. A safety ratio (hERG IC₂₀/EC₅₀)>30 suggests a sufficient window to differentiate the pharmacology by inhibiting TLR7/8/9 pathways from the potential hERG related cardiotoxicity.

TABLE 2
hERG and safety ratio results

| | | | hERG | hERG | hERG |
|---|---|---|---|---|---|
| | hERG | hERG | IC₂₀/ | IC₂₀/ | IC₂₀/ |
| | IC₂₀ | IC₅₀ | TLR7 | TLR8 | TLR9 |
| Example No | (μM) | (μM) | IC₅₀ | IC₅₀ | IC₅₀ |
| 4 | 4.2081 | >10.0000 | 471.9 | 1448.2 | 27.2 |
| 15 | 8.3630 | >20.0000 | 2144.2 | 2090 | 38.7 |
| 20 | >10.000 | >20.0000 | >1666.7 | >636.9 | >44.2 |
| 25 | 5.7822 | >20.0000 | 837.6 | 516.0 | 62.8 |

### Example 31

### Human PBMC Cell-Based Assay

Unlike the HEK reporter cell lines, human peripheral blood mononuclear cell (PBMC) represents primary human immune cells in blood mainly consisting of lymphocytes, monocytes, and dendritic cells. These cells express TLR7, TLR8, or TLR9, and therefore are natural responders to respective ligand stimulation. Upon activation of these TLRs, PBMCs secrete similar cytokines and chemokines in vitro and in vivo, and therefore the in vitro potency of a TLR7/8/9 antagonist in human PBMC is readily translatable to its pharmacodynamics response in vivo.

Human peripheral blood mononuclear cells (PBMC) were isolated from freshly-drawn lithium-heparinized (Lithium Heparin Plus blood Collection tube, BD Vacutainer®) healthy donor whole blood by density gradient (Ficoll-Paque™ PLUS, GE Healthcare life Sciences). Briefly, 50 mL of blood was diluted with 25 mL PBS (without Ca²⁺, Mg²⁺) in a 50 mL conical tube with porous barrier (Leucosep tube, Greiner bio-one), where 15.5 mL Ficoll-Paque was under laid after spinning. Tubes were centrifuged for 20 minutes at 800×g (1946 rpm) with the brake in the off position, and PBMC were collected from the buffy coat. Cells were then washed twice in PBS, and red blood cells were lysed by suspension in 2 mL (Red Blood Cell Lysis Buffer, Alfa Aesar) for 5-10 minutes at room temperature. After a final wash in PBS, PBMC were resuspended at a final concentration of 2×10⁶ cells/mL in RPMI-1640 media with GlutaMAX™ (Gibco) supplemented with 10% Fetal Bovine Serum (Sigma) and plated at 150 μL/well (3×10⁵ cells/well) in tissue culture treated round bottom 96-well plates (Corning Incorporated). Antagonist compounds (compounds of this invention) solubilized and serial diluted in 100% DMSO were added in duplicate to cells to yield a final concentration of 1% DMSO (v/v). PBMC were incubated with antagonist compounds for 30 minutes at 37° C., 5% CO₂ before adding various TLR agonist reagents in 48 μL complete media per well as follows (final concentrations indicated): CpG ODN 2216 (InvivoGen) at 1 μM for TLR9, ORN 06/LyoVec (InvivoGen) at 1 μg/mL for TLR8 and R848 (InvivoGen) at 1 μg/mL for TLR7 and TLR8. PBMC were incubated overnight at 37° C. with 5% CO₂. Cell culture supernatants were collected, and levels of various human cytokines were assessed by Luminex assay (ProcartaPlex™ Multiplex Immunoassay, Invitrogen) or ELISA procedure according to the manufacturer's recommended protocol (eBioscience, ThermoFisher Scientific). Viability of the cells was also checked with Cell Viability Assay (CellTiter Glo®Luminescent Cell Viability Assay, Promega).

TABLE 3
hPBMC results

| Example No | hPBMC/TLR9 IC₅₀ (μM) | hPBMC/TLR78 IC₅₀ (μM) |
|---|---|---|
| 9 | 0.50 | 0.008 |
| 12 | 0.29 | 0.016 |
| 14 | 0.25 | |
| 15 | 0.47 | |
| 17 | 0.10 | |
| 20 | 0.50 | |
| 25 | 0.13 | 0.0045 |

### Example 32

### Human Microsome Stability Assay

The human microsomal stability assay is used for early assessment of metabolic stability of a test compound in human liver microsomes.

Human liver microsomes (Cat. NO.: 452117, Corning, USA; Cat. NO.: H2610, Xenotech, USA) were preincubated with test compound for 10 minutes at 37° C. in 100 mM potassium phosphate buffer, pH 7.4. The reactions were initiated by adding NADPH regenerating system. The final incubation mixtures contained 1 μM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 μL of cold acetonitrile (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The compounds of present invention showed good human liver microsome stability determined in the above assay, results are shown in Table 4 below.

TABLE 4
Human liver microsome stability of
the compounds of present invention

| | Clearance of Human |
|---|---|
| Example No | microsome (mL/min/kg) |
| 1 | 9.06 |
| 3 | 6.15 |
| 4 | 8.25 |
| 9 | 7.20 |
| 10 | 9.15 |
| 11 | 7.02 |
| 12 | 6.15 |
| 13 | 9.16 |
| 14 | 9.45 |
| 15 | 6.15 |
| 16 | 9.54 |
| 22 | 6.15 |
| 25 | 6.15 |
| 26 | 8.69 |
| 27 | 7.75 |

### Example 33

### 3T3 In Vitro Phototoxicity Assay

Phototoxicity is defined as a toxic response that is elicited after the first exposure of the skin to certain chemicals and subsequent exposure to light, or that is induced similarly by skin irradiation after systemic administration of a chemical substance. The assay used in this study is designed to detect the phototoxic potential of a chemical by using a simple in vitro cytotoxicity assay with Balb/c 3T3 mouse fibroblasts. The principle of this test is a comparison of the cytotoxicity of a chemical when tested with and without exposure to a non-toxic dose of UVA-light. Cytotoxicity is expressed as a dose dependent reduction of the growth rate of cells as determined by uptake of the vital dye Neutral Red one day after treatment.

1. Method

### Preparation of Stock Solution and Dosage of Test Item

A small amount of substance was weighed and formulated freshly in DMSO just before the start of the exposure of the cells. This stock solution or appropriate dilutions with DMSO were added to the cell suspensions to obtain the required final concentrations. All solutions were generally prepared in Eppendorf caps and discarded after use.

### Reference Substance

Chlorpromazine (HCL) (Sigma, Batch/Lot No.: 120M1328V), test concentration: 300 μg/mL, Solvent: PBS/3% DMSO
Measurement of UV absorption spectrum

The absorption spectra as such or with UV-A or with UV-B pre-irradiation were recorded between 240 nm and 400 nm with a Lambda-2 spectral photometer (Perkin Elmer).

| | | | |
|---|---|---|---|
| UV | for UV-A: | Sol 500 with filter H1 | |
| radiation | | Main spectrum: | 315-690 nm |
| sources: | | Irradiance: | approx. 1.67 mW/cm² |
| | | Radiation dose: | approx. 5 J/cm² |
| | for UV-B: | Philips TL 20 W/12 | |
| | | Main spectrum: | 290-320 nm |
| | | Irradiance: | approx. 0.083 mW/cm² |
| | | Radiation dose: | approx. 0.05 J/cm² |

### Determination of Phototoxicity

For this study the Neutral Red uptake (NRU) assay of Borenfreund and Puerner (Borenfreund, E, Puerner J A. Toxicity determined in vitro by morphological alterations and Neutral Red absorption. Toxicology Lett. 1985; 24:119-124) modified according to INVITTOX protocol No 78 (ERGATT/FRAME data bank of in vitro techniques in toxicology. INVITTOX PROTOCOL No 78. 3T3 NRU Phototoxicity Assay. March 1994) has been adapted to examine a possible phototoxic potential of the test item. This assay is based on the active uptake of the Neutral Red dye into the lysosomes of cultured murine fibroblasts. Because lysosomal membranes are known to be a site of action of many phototoxic compounds, this assay can provide a measure of potential for phototoxic injury.

### Preparation of Cell Culture

A murine fibroblasts clone A 31 (ATCC no. CCL 163—passage No. 108) were cultured in 175 cm² tissue culture grade flasks, containing sDMEM (Dulbecco's Minimal Essential Medium, supplemented with 10% fetal calf serum, 2 mM L-glutamine, 100 units/ml Penicillin and 100 μg/ml streptomycin) at 37° C. in a humidified atmosphere of 6% CO₂. Before cells approach confluence they were removed from flasks by trypsinisation. Prior to use in an assay, the cells were transferred to 96-well microtiter plates at a concentration of 1×10⁴ cells/well in 100 ul volumes of sDMEM and allowed to attach for 24 h.

### Exposure to Test Item

For incubation with murine fibroblasts, the test item was diluted in PBS/3% DMSO (detailed concentrations see in results).

Culture medium (Dulbecco's Modified Eagle Medium(DMEM), GlutaMAX (Gibco Ref 21885-025), 10% Fetal Bovine Serum (FBS) (Gibco Ref 10270-106), 100 IU/ml Penicillin and 100 μg/ml Streptomycin (Gibco Ref 15140-122)) was removed from the wells and murine fibroblasts were washed with PBS. Afterwards 100 μL of PBS/3% DMSO containing the test item was added and target cells were incubated for 1 h at 37° C. with 6% CO₂.

### UV Exposure

For each test item the microtiter plates were prepared according to Table 6. “UVA plates” were exposed to approx. 5 J/cm² UVA light, the “Dark plates” were kept in the dark and served as cytotoxicity control. Plates with chlorpromazine hydrochloride served as positive control. UV flux was measured with a UV-meter (Dr. Gröbel RM21).

Following UV irradiation, the test item was removed from the wells (one washing step with PBS) and replaced with sDMEM. Target cells were then incubated overnight at 37° C. in 6% CO₂.

TABLE 5
96-well microtiter plate setup

| | 1 | 2 | 3 | 4 | 5 | 6 | 7 | 8 | 9 | 10 | 11 | 12 |
|---|---|---|---|---|---|---|---|---|---|---|---|---|
| A | S1 | S2 | U01 | U02 | U03 | U04 | U05 | U06 | U07 | U08 | S2 | S1 |
| B | S1 | S2 | | | | | | | | | S2 | S1 |
| C | S1 | S2 | | | | | | | | | S2 | S1 |
| D | S1 | S2 | | | | | | | | | S2 | S1 |
| E | S1 | S2 | | | | | | | | | S2 | S1 |
| F | S1 | S2 | | | | | | | | | S2 | S1 |
| G | S1 | S2 | | | | | | | | | S2 | S1 |
| H | S1 | S2 | | | | | | | | | S2 | S1 |

96-well microtiter plates were prepared as follows:

Each plate contained wells with cells and solvent but without test item which were either not incubated with Neutral Red solution (0% standard—51) or were stained with Neutral Red (100% standard—S2) for calculation of the standard cell viability curve. Wells labeled with U01-U08 contained the different test item concentrations.

### Neutral Red Uptake

The ready to use Neutral Red (NR) staining solution was freshly prepared as follows:
0.4% aqueous stock solution was shielded from light and filtered before use to remove NR crystals.
1:40 dilution of the stock solution was then prepared in sDMEM and added to the cells.

After the incubation the wells to be assayed were filled with 100 μL of the sDMEM containing Neutral Red. The target cells were incubated with the NR for 3 h at 37° C. in 6% CO₂.

Measurement of Neutral Red uptake

Unincorporated Neutral Red was removed from the target cells and the wells washed with at least 100 μL of PBS. 150 μL of Neutral Red desorb solution (1% glacial acetic acid, 50% ethanol in aqua bidest) was then added to quantitatively extract the incorporated dye. After at least 10 mins of vigorous shaking of the plates on a microtiter plate shaker until Neutral Red has been extracted from the cells and formed a homogeneous solution, the absorption of the resulting colored solution was measured with a SPECTRAmax PLUS microtiter plate reader (Molecular Devices) at 540 nm.

### Calculation of Cell Viability

Cell viability was calculated with the SOFTmax Pro software package (Molecular Devices). First a two-point standard curve (0% and 100% viability) was calculated with the linear curve fit option of the program based on the following formula:

Y=A+(B×X)

(A=y-intercept of the line; B=slope of the line;
0% cell viability=cells with solvent, but without test item and Neutral Red;
100% cell viability=cells with solvent and Neutral Red, but without test item)

By this means the viability of the cells incubated with increasing concentrations of the test chemical was calculated. Chlorpromazine (HCl) served as positive control in the experiment.

### Calculation of IC₅₀ Values

All calculations were performed with the SOFTmax Pro analysis software package (Molecular Devices—for details see: http://www.mbl.edu/jbpc/files/2014/05/SoftMax_Pro_User_Guide.pdf)

### Calculation of Discrimination Factor for Phototoxicity

For evaluation of phototoxic potential, the IC₅₀ values determined with and without UV exposure were compared.

Factor=IC₅₀(−UV)/IC₅₀(+UV)

For discrimination between phototoxic and non-phototoxic test chemicals a cut-off factor of >5 was applied (Liebsch M, Spielmann H, Balls M, Brand M, Döring B, Dupuis J, Holzhüter H G, Klecak G, L. Eplattenier H, Lovell W, Maurer T, Moldenhauer F, Moore L, Pape W, Pfannenbecker U, Potthast J M, De Silva O, Steiling W, Willshaw A. First results of the EC/COLIPA Validation Project. In Vitro Phototoxicity Testing. In: In Vitro Skin Toxicology: Irritation, Phototoxicity, Sensitization; Vol. 10. Alternative Methods in Toxicology,-Eds. Rougier A, Maibach H I, Goldberg A M; Mary Ann Liebert Publ.: New York, USA 1994, pp. 243-251).

Test items which are not cytotoxic to murine fibroblasts even at the highest concentrations tested, but show a strong dose dependent decrease in cell viability after UV exposure are considered also phototoxic (Spielmann H, Balls M, Dupuis J, Pape W J W, Pechovitch G, Silva DeO, Holzhütter, H G, Clothier R, Desolle P, Gerberick F, Liebsch M, Lowell W W, Maurer T, Pfannenbecker U, Potthast J M, Csato M, Sladowski D, Steiling W, Brantom P. The international EU/COLIPA in vitro phototoxicity validation study: Results of phase II (blind trial). Part 1: The 3T3 NRU phototoxicity test. Toxicology in Vitro 1998, 12: 305-327).

The test results were shown below, the compounds of this invention showed very good phototoxicity profile.

TABLE 6
The 3T3 test results for the compound of this invention

| Example No | Phototoxicity factor | IC₅₀ (UV-A) (μg/mL) |
|---|---|---|
| 14 | 1 | 100 |

### Example 34

### Parallel Artificial Membrane Permeability Assay (PAMPA)

PAMPA (Parallel Artificial Membrane Permeability Assay) is a first-line permeability screen for drug candidates. This assay mimics the transcellular absorption conditions using an artificial phospholipid membrane and generates a permeability value that can be used for compound ranking and optimization as well as input parameters for in silico models to predict intestinal absorption.

Permeation experiments are carried out in hydrophobic PVDF 96-well microtiter filter plates (MultiScreen Filter Plate, Millipore, #MAIPN4550). Each well is coated with PVDF membrane, which is prepared with 5 μL Dodecane (Sigma, D221104) that contains 1% lecithin (Sigma, P3556-1G).

The typical PAMPA experimental protocol is as follows: The donor plate is placed on a Teflon acceptor plate that has been pre-filled with 150 μL of 100 mM PBS buffer (2.6 g KH₂PO₄ and 18.5 g K₂HPO₄·3H₂O are dissolved in about 1000 mL of ultra-pure water and mixed thoroughly. The pH is adjusted to 7.40±0.05, using either 1 M sodium hydroxide or 1M hydrochloric acid) containing 5% DMSO. The filter on the bottom of each acceptor well is filled with 300 μL of 100 mM PBS buffer (2.6 g KH₂PO₄ and 18.5 g K₂HPO₄·3H₂O are dissolved in about 1000 mL of ultra-pure water, mixed thoroughly. The pH was adjusted to 7.40±0.05, using either 1 M sodium hydroxide or 1M hydrochloric acid). The resulting sandwich is incubated at room temperature under constant shaking (300 rpm) for 4 hours. The sandwich is then disassembled. Before incubation, spike 20 μL dosing solution and mix with 250 μL PBS and 130 μL quench solution (acetonitrile) as TO sample. After incubation, collect 270 μL solutions from acceptor chamber, followed by the addition of 130 μL acetonitrile. Collect 20 μL solution from donor chamber and add 250 μL PBS and 130 μL acetonitrile. The concentration of compound in all samples are determined by LC-MS/MS and the equations for determining the permeability (Pe, 10⁻⁶ cm/s) are as follows.

$${P}_{e} = - Cx ln ( 1 - \frac{( {V}_{D} + {V}_{R} ) \times {C}_{R}}{{V}_{D} \times {C}_{D} + {V}_{R} \times {C}_{R}} ) ,$$

$$where$$

$$C = \frac{{V}_{D} \times {V}_{R}}{( {V}_{D} + {V}_{R} ) \times Area \times Time}$$

$$\% Solution Recovery = \frac{{C}_{R} \times {V}_{R} + {C}_{D} \times {V}_{D}}{{C}_{0} \times {V}_{D}} \times 100$$

V_{D} is the volume of the donor well; V_{R} is the volume of the acceptor well; Area is the active surface area of membrane; Time is the incubation time (14,400 s in this assay); C_{R} and C_{D} are the concentrations of compound in acceptor and donor solutions, respectively, at the completion of the assay; C₀ is the concentration of compound in donor solution before incubation.

The main readout of the PAMPA assay is the permeability value Pe expressed in 10⁻⁶ cm/s. Secondary readouts determined are the amounts of compound in the donor and acceptor compartments as well as compound retention in the membrane. Depending on the permeation rate and the membrane retention, compounds are classified as “low” (Pe<0.2 and membrane retention<20%) or “medium & high” (Pe>=0.2; or Pe<0.2 and membrane retention>=20%). Each sample is measured in triplicate; the standard deviation is determined for the permeation constant Pe. No results are displayed when the sample in acceptor and donor solutions reaches equilibrium (no kinetic information), when the reference is precipitated (turbidity measurement), or in case of analytical limitations.

TABLE 7
Parallel Artificial Membrane Permeability Assay (PAMPA)

| | PAMPA Pe | Membrane Retention | PAMPA |
|---|---|---|---|
| Example No | (10⁻⁶ cm/s) | (%) | category |
| 2 | 0.87 | 22.73 | medium & high |
| 3 | 0.8 | 20.3 | medium & high |
| 4 | 1.2 | 17.52 | medium & high |
| 5 | 2.1 | 17.82 | medium & high |
| 7 | 1.2 | 20.4 | medium & high |
| 8 | 0.85 | 34.49 | medium & high |
| 9 | 0.88 | 49 | medium & high |
| 10 | 0.81 | 17.7 | medium & high |
| 11 | 0.84 | 9.47 | medium & high |
| 12 | 1 | 7.03 | medium & high |
| 21 | 0.73 | 28 | medium & high |
| 22 | 0.5 | 26.45 | medium & high |

### Example 35

### Single Dose Pharmacokinetics (PK) Study in Male Wister-Han Rats

Pharmacokinetic properties of selected compounds were assessed by single dose PK studies in Male Wister-Han Rats (vendor: Beijing Vital River Laboratory Animal Technology Co., Ltd). Briefly, two groups of animals were administered a single dose of respective compound intravenously (IV, bolus) at 2 mg/kg or orally (PO, by gavage) at 10 mg/kg. Blood samples (approximately 150 μL) were collected via Jugular vein at 5 min (only for IV), 15 min, 30 min, 1 h, 2 h, 4 h, 7 h and 24 h post-dose. Blood samples were placed into tubes containing EDTA-K₂ anticoagulant and centrifuged at 3000 rpm for 15 min at 4° C. to separate plasma from the samples. After centrifugation, the resulting plasma was transferred to clean tubes for bioanalysis with LC/MS/MS. The pharmacokinetic parameters were calculated using non-compartmental analysis. The volume of distribution (V_{SS}), half-life (T_{1/2}) and clearance (CL) were obtained based on the plasma concentration-time curve after IV dose. The peak concentration (Cₘₐₓ) was recorded directly from experimental observations after PO dose. The area under the plasma concentration-time curve (AUC₀₋ₗₐₛₜ) was calculated using the linear trapezoidal rule up to the last detectable concentration. The bioavailability (F) was calculated based on the dose normalized AUC₀₋ₗₐₛₜ after IV and PO dose.

The V_{SS} of a drug represents the degree to which a drug is distributed in body tissue rather than the plasma. V_{SS} is directly proportional with the amount of drug distributed into tissue. A higher V_{SS} indicates a greater amount of tissue distribution.

Results of PK parameters following IV and PO administration are given in Table 9.

TABLE 8
PK parameters for the compounds of this invention

| Example | PO Cₘₐₓ | PO AUC₀₋ₗₐₛₜ | IV AUC₀₋ₗₐₛₜ | CL | Vss | T_{1/2} | |
|---|---|---|---|---|---|---|---|
| No | (ng/mL) | (h × ng/mL) | (h × ng/mL) | (mL/min/kg) | (L/kg) | (h) | F (%) |
| 10 | 257 | 3643 | 1055 | 28 | 15 | 8.6 | 69 |

### Example 36

### Human Cytosolic Aldehyde Oxidase (AO) Substrate Assay

The Human Cytosolic AO Substrate Assay is to assess the metabolic stability of test compound in human liver cytosol with and without selected aldehyde oxidase (AO) inhibitor. Cytosolic incubations were carried out in deep-well 96-well plates. The conversion of test compound and the formation of oxidized metabolite were monitored over a 60 minutes time period. The volume for incubation was 0.4 mL/well and time points were 0.5, 3.5, 6.5, 10, 20, 30, 45 & 60 minutes. The human liver cytosol (1 mg protein/mL, BD UltraPool™ Human Cytosol) and test compound (1 μM in duplicate) or control compound (i.e. known AO substrates; 1 μM in duplicate) were incubated at 37° C. in a water bath. At each corresponding time point, 120 μL of quenching solution (Hydralazine in acetonitrile, 50 the total organic concentration will be ≤1% in the final incubation) was added to stop the reaction, and a 40 μL sample was withdrawn. All sample plates were mixed well and centrifuged at 3220×g for 10-20 minutes, and supernatants were diluted with water or buffers as appropriate for LC/MS/MS analysis.

In order to determine the in vitro elimination rate of the test compounds and control compound, the analyte/internal standard peak area ratios were converted to percentage remaining with the following equation:

$$\% Remaining = \frac{Peak area ratio of analyte to internal standard at each time point}{Peak area ratio of analyte to internal standard at t = 0} \times 100 \%$$

And half-life (minute) was calculated from a log linear plot of % Remaining versus time. The estimation of the hepatic intrinsic clearance (CLint) in vitro values were calculated from substrate disappearance rate in liver cytosol incubations as follows: CLint (cytosol)=0.693/half-life/mg cytosol protein per mL.

TABLE 9
AO parameters for the compounds of this invention

| | | | |
|---|---|---|---|
| | Clint | T_{1/2} | Clint Ratio to |
| Example No | (μL/min/mg) | (min) | Carbazeran |

* Carbazeran is an Aldehyde oxidase (AO) substrate and a phosphodiesterase inhibitor that produces concentration-dependent positive inotropic responses. In humans, the compound is almost completely cleared via 4-hydroxylation to the phthalazinone metabolite by AO.

1. A compound of formula (I), 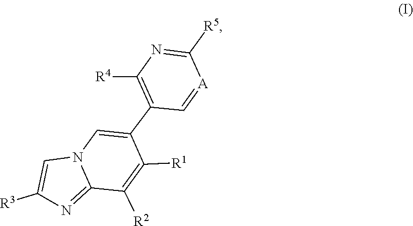
wherein
R¹ is H or C₁₋₆alkyl;
R² is C₁₋₆alkyl or C₁₋₆alkoxy;
R³ is H or C₁₋₆alkyl;
R⁴ is H, C₁₋₆alkyl, haloC₁₋₆alkyl, C₁₋₆alkoxy or (C₁₋₆alkyl)₂amino;
R⁵ is (5,6,7,8-tetrahydropyrido[3,4-d]pyrimidinyl)piperazinyl; (amino(C₁₋₆alkoxy)pyrrolidinyl)piperidinyl; (C₁₋₆alkylpiperazinyl)piperidinyl; (hydroxyC₁₋₆alkyl)piperazinyl; (morpholinylcarbonyl)piperazinyl; 1,3,4,6,7,8,9,9a-octahydropyrazino[1,2-a]pyrazinyl; 3,9-diazaspiro[5.5]undecanyl; 4-oxa-1,9-diazaspiro[5.5]undecanyl; 5-oxa-2,8-diazaspiro[3.5]nonanyl; amino(C₁₋₆alkoxy)pyrrolidinyl; amino(C₁₋₆alkyl)azetidinyl; amino(C₁₋₆alkyl)piperidinyl; aminopiperidinyl or piperazinylpiperidinyl;
A is CH, N or CR⁶, wherein R⁶ is C₁₋₆alkyl;
or a pharmaceutically acceptable salt thereof.

2. A compound according to claim 1, wherein A is CH or N.

3. A compound according to claim 1 or 2, wherein R¹ is H.

4. A compound according to any one of claims 1 to 3, wherein R³ is H.

5. A compound according to claim 4, wherein R⁴ is C₁₋₆alkyl, haloC₁₋₆alkyl or C₁₋₆alkoxy.

6. A compound according to claim 5, wherein R⁵ is (amino(C₁₋₆alkoxy)pyrrolidinyl)piperidinyl; 3,9-diazaspiro[5.5]undecanyl; amino(C₁₋₆alkoxy)pyrrolidinyl; piperazinylpiperidinyl or 5-oxa-2,8-diazaspiro[3.5]nonanyl.

7. A compound according to claim 6, wherein R⁵ is (3-amino-4-methoxy-pyrrolidin-1-yl)-1-piperidinyl; 3,9-diazaspiro[5.5]undecan-3-yl; 4-methoxy-3-amino-pyrrolidin-1-yl; 4-piperazin-1-yl-1-piperidinyl or 5-oxa-2,8-diazaspiro[3.5]nonan-2-yl.

8. A compound according to claim 1, wherein
R¹ is H;
R² is C₁₋₆alkyl or C₁₋₆alkoxy;
R³ is H;
R⁴ is C₁₋₆alkyl, haloC₁₋₆alkyl or C₁₋₆alkoxy;
R⁵ is (amino(C₁₋₆alkoxy)pyrrolidinyl)piperidinyl; 3,9-diazaspiro[5.5]undecanyl; amino(C₁₋₆alkoxy)pyrrolidinyl; piperazinylpiperidinyl or 5-oxa-2,8-diazaspiro[3.5]nonanyl;
A is CH or N;
or a pharmaceutically acceptable salt thereof.

9. A compound according to claim 8, wherein
R¹ is H;
R² is methyl or methoxy;
R³ is H;
R⁴ is ethyl, difluoromethyl or methoxy;
R⁵ is (3-amino-4-methoxy-pyrrolidin-1-yl)-1-piperidinyl; 3,9-diazaspiro[5.5]undecan-3-yl; 4-methoxy-3-amino-pyrrolidin-1-yl; 4-piperazin-1-yl-1-piperidinyl or 5-oxa-2,8-diazaspiro[3.5]nonan-2-yl;
A is CH or N;
or a pharmaceutically acceptable salt thereof.

10. A compound selected from:
1-[6-isopropyl-5-(8-methylimidazo[1,2-a]pyridin-6-yl)-2-pyridyl]piperidin-4-amine;
6-[2-isopropyl-6-(4-piperazin-1-yl-1-piperidyl)-3-pyridyl]-8-methyl-imidazo[1,2-a]pyridine;
6-[2-isopropyl-6-(4-piperazin-1-yl-1-piperidyl)-3-pyridyl]-2,8-dimethyl-imidazo[1,2-a]pyridine;
1-[5-(2,8-dimethylimidazo[1,2-a]pyridin-6-yl)-6-isopropyl-2-pyridyl]piperidin-4-amine;
[(2S)-1-[5-(2,8-dimethylimidazo[1,2-a]pyridin-6-yl)-6-isopropyl-2-pyridyl]piperazin-2-yl]methanol;
9-[5-(2,8-dimethylimidazo[1,2-a]pyridin-6-yl)-6-isopropyl-2-pyridyl]-4-oxa-1,9-diazaspiro[5.5]undecane;
6-[2-isopropyl-6-(4-piperazin-1-yl-1-piperidyl)-3-pyridyl]-7,8-dimethyl-imidazo[1,2-a]pyridine;
2-[5-(2,8-dimethylimidazo[1,2-a]pyridin-6-yl)-6-isopropyl-2-pyridyl]-1,3,4,6,7,8,9,9a-octahydropyrazino[1,2-a]pyrazine;
8-methyl-6-[5-methyl-6-[4-(4-methylpiperazin-1-yl)-1-piperidyl]-3-pyridyl]imidazo[1,2-a]pyridine;
6-[2-(difluoromethyl)-6-(4-piperazin-1-yl-1-piperidyl)-3-pyridyl]-8-methyl-imidazo[1,2-a]pyridine;
6-[2-(difluoromethyl)-6-(4-piperazin-1-yl-1-piperidyl)-3-pyridyl]-2,8-dimethyl-imidazo[1,2-a]pyridine;
1-[1-[6-(difluoromethyl)-5-(2,8-dimethylimidazo[1,2-a]pyridin-6-yl)-2-pyridyl]-4-piperidyl]-3-methyl-azetidin-3-amine;
3-[6-(difluoromethyl)-5-(8-methoxyimidazo[1,2-a]pyridin-6-yl)-2-pyridyl]-3,9-diazaspiro[5.5]undecane;
2-[1-[6-ethyl-5-(8-methylimidazo[1,2-a]pyridin-6-yl)-2-pyridyl]-4-piperidyl]-5-oxa-2,8-diazaspiro[3.5]nonane;
(3R,4R)-1-[1-[6-ethyl-5-(8-methylimidazo[1,2-a]pyridin-6-yl)-2-pyridyl]-4-piperidyl]-4-methoxy-pyrrolidin-3-amine;
2-[1-[5-(2,8-dimethylimidazo[1,2-a]pyridin-6-yl)-6-ethyl-2-pyridyl]-4-piperidyl]-5-oxa-2,8-diazaspiro[3.5]nonane;
(3R,4R)-1-[1-[5-(2,8-dimethylimidazo[1,2-a]pyridin-6-yl)-6-ethyl-2-pyridyl]-4-piperidyl]-4-methoxy-pyrrolidin-3-amine;
[4-[5-(2,8-dimethylimidazo[1,2-a]pyridin-6-yl)-6-ethyl-2-pyridyl]piperazin-1-yl]-morpholin-2-yl-methanone;
2-[4-[5-(2,8-dimethylimidazo[1,2-a]pyridin-6-yl)-6-ethyl-2-pyridyl]piperazin-1-yl]-5,6,7,8-tetrahydropyrido[3,4-d]pyrimidine;
1-[5-(2,8-dimethylimidazo[1,2-a]pyridin-6-yl)-6-ethyl-2-pyridyl]-4-methyl-piperidin-4-amine;
6-[2-ethyl-6-(4-piperazin-1-yl-1-piperidyl)-3-pyridyl]-2,8-dimethyl-imidazo[1,2-a]pyridine;
3-[3-ethyl-5-(8-methylimidazo[1,2-a]pyridin-6-yl)-2-pyridyl]-3,9-diazaspiro[5.5]undecane;
N,N-dimethyl-3-(8-methylimidazo[1,2-a]pyridin-6-yl)-6-(4-piperazin-1-yl-1-piperidyl)pyridin-2-amine;
6-[4-[(3R,4R)-3-amino-4-methoxy-pyrrolidin-1-yl]-1-piperidyl]-N,N-dimethyl-3-(8-methylimidazo[1,2-a]pyridin-6-yl)pyridin-2-amine;
6-[2-methoxy-6-(4-piperazin-1-yl-1-piperidyl)-3-pyridyl]-8-methyl-imidazo[1,2-a]pyridine;
(3R,4R)-1-[1-[4-ethyl-5-(8-methylimidazo[1,2-a]pyridin-6-yl)pyrimidin-2-yl]-4-piperidyl]-4-methoxy-pyrrolidin-3-amine;
6-[4-ethyl-2-(4-piperazin-1-yl-1-piperidyl)pyrimidin-5-yl]-8-methyl-imidazo[1,2-a]pyridine; and
2-[1-[4-ethyl-5-(8-methylimidazo[1,2-a]pyridin-6-yl)pyrimidin-2-yl]-4-piperidyl]-5-oxa-2,8-diazaspiro[3.5]nonane;
or a pharmaceutically acceptable salt thereof.

11. A process for the preparation of a compound according to any one of claims 1 to 10 comprising any of the following steps:
a) Deprotection of compound of formula (IX), 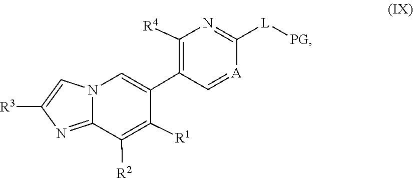
with an acid to afford compound of formula (I-1), 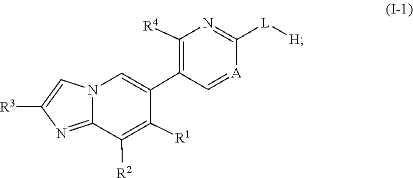
b) Deprotection of compound of formula (XI), 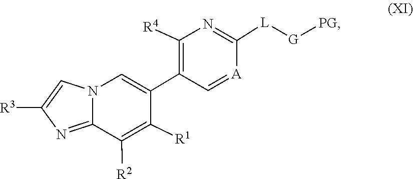
with an acid to afford compound of formula (I-2), 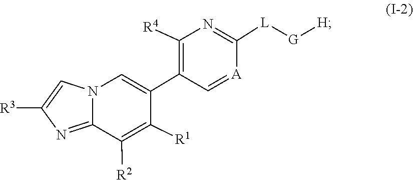
c) Deprotection of compound of formula (XV), 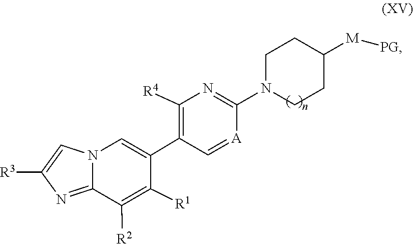
with an acid to afford compound of formula (I-3), 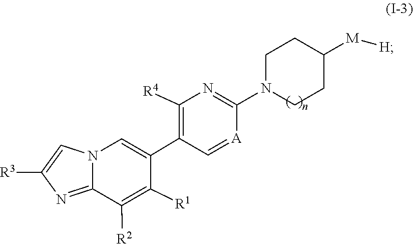
wherein
PG is a protecting group;
L is unsubstituted or substituted group selected from piperazinyl, piperidinyl, 1,3,4,6,7,8,9,9a-octahydropyrazino[1,2-a]pyrazinyl, 3,9-diazaspiro[5.5]undecanyl, 4-oxa-1,9-diazaspiro[5.5]undecanyl, 5-oxa-2,8-diazaspiro[3.5]nonanyl, azetidinyl and pyrrolidinyl;
G is 5,6,7,8-tetrahydropyrido[3,4-d]pyrimidinyl, amino(C₁₋₆alkoxy)pyrrolidinyl, C₁₋₆alkylpiperazinyl and piperazinyl;
M is amino(C₁₋₆alkoxy)pyrrolidinyl, C₁₋₆alkylpiperazinyl or piperazinyl;
n is 0, 1 or 2;
in step a), b) and c) the acid is TFA;
R¹ to R⁶ and A are defined as in any one of claims 1 to 10.

12. A compound or pharmaceutically acceptable salt according to any one of claims 1 to 10 for use as therapeutically active substance.

13. A pharmaceutical composition comprising a compound in accordance with any one of claims 1 to 10 and a therapeutically inert carrier.

14. The use of a compound according to any one of claims 1 to 10 for the treatment or prophylaxis of systemic lupus erythematosus or lupus nephritis.

15. The use of a compound according to any one of claims 1 to 10 for the preparation of a medicament for the treatment or prophylaxis of systemic lupus erythematosus or lupus nephritis.

16. The use of a compound according to any one of claims 1 to 10 for the preparation of a medicament for TLR7 and TLR8 and TLR9 antagonist.

17. A compound or pharmaceutically acceptable salt according to any one of claims 1 to 10 for the treatment or prophylaxis of systemic lupus erythematosus or lupus nephritis.

18. A compound or pharmaceutically acceptable salt according to any one of claims 1 to 10, when manufactured according to a process of claim 11.

19. A method for the treatment or prophylaxis of systemic lupus erythematosus or lupus nephritis, which method comprises administering a therapeutically effective amount of a compound as defined in any one of claims 1 to 10.

20. The invention as hereinbefore described.
